(19) United States
(12) Patent Application Publication
(10) Pub. No.: US 2023/0302056 A1
(43) Pub. Date: Sep. 28, 2023

(54) ALLOGENEIC COMPOSITION FOR THE TREATMENT OF COVID-19

(71) Applicant: NEXTCELL PHARMA AB, Huddinge (SE)

(72) Inventors: Mathias SVAHN, Nacka (SE); Johanna DAHLLUND, Tullinge (SE); Bahareh KHALAJ, Sundbyberg (SE); Lindsay Catrina DAVIES, Munich (DE)

(21) Appl. No.: 18/041,507

(22) PCT Filed: Aug. 13, 2021
(86) PCT No.: PCT/EP2021/072621

§ 371 Date: Feb. 13, 2023

(30) Foreign Application Priority Data
Aug. 14, 2020 (WO) PCT/EP2020/072918

(51) Int. Cl. A61K 35/28; A61P 37/02; C12N 5/0775; A61P 31/14; G01N 33/50

(52) U.S. Cl. CPC A61K 35/28; A61P 31/14; A61P 37/02; C12N 5/0668; G01N 33/5023

(57) ABSTRACT

The present disclosure relates to the use of allogeneic populations of mesenchymal stem/stromal cells and related compositions, which populations and compositions comprise cells pooled from multiple donors, in the treatment and/or prevention of COVID-19 infection or for use in the treatment and/or prevention of symptoms associated with COVID-19 infection. The present disclosure also relates to methods for obtaining said compositions.

### TECHNICAL FIELD

The present disclosure relates to the use of allogeneic populations of mesenchymal stem/stromal cells and related compositions, which populations and compositions comprise cells pooled from multiple donors, in the treatment and/or prevention of COVID-19 infection or for use in the treatment and/or prevention of symptoms associated with COVID-19 infection. The present disclosure also relates to methods for obtaining said compositions.

### BACKGROUND

Mesenchymal stem cells (MSCs) are non-hematopoietic cells expressing the surface markers CD73, CD90, and CD105 while lacking the expression of CD14, CD34, and CD45. When expanded as polyclonal cultures, they are a heterogenous population of cells with retained capacity for self-renewal and differentiation into various forms of mesenchyme (Dominici, et al. (2006), Cytotherapy 8: 315-317). In vitro, MSCs adhere to plastic under standard tissue culture conditions, and have the capacity to differentiate into osteoblasts, adipocytes, and chondroblasts. MSCs can be found not only in bone marrow, in which they were originally found, but also in almost all other forms of tissues e.g., Wharton’s jelly and the placenta. For example, Wharton Jelly derived MSCs have homing capabilities, which induces them to travel to inflammatory sites and locally affects the inflammatory/ immune-mediated tissue damage. The primary mode of action of MSCs include release of paracrine or endocrine factors which create an environment facilitating and stimulating endogenous repair. Hallmark processes regulated by MSCs are that they are contributing to an environment for endogenous repair or regeneration including immunomodulation, stimulation of proliferation of resident tissue cells or local progenitor cells.

Coronavirus -19 (COVID-19) is the third documented example of a species crossing virus into humans. Corona viruses are a family of viruses primarily associated with infections of the upper respiratory tract, including SARS-CoV, MERS-CoV and SARS-CoV-2 (Li Y. Zhou W, Yang L, You R. Pharmacol Res. 2020; 157:104833). Patients that are infected with SARS-CoV-2 (COVID-19) present with classic flu symptoms with a range of symptoms including fever and dry cough. Disruption to the respiratory system is common with this disease, including development of pneumonia and acute respiratory distress syndrome (ARDS) in severe cases. Increasing evidence also suggest the prevalence and relevance of non-respiratory symptoms including anosmia, dysgeusia, neurological symptoms including stroke, vascular inflammation and multisystem inflammation.

SARS-CoV-2 enters the body mainly through the inhalation of airborne droplets, infiltrating the nasal and buccal cavities and thereby gaining access to the respiratory tract. Primary infection of the host occurs through the epithelium lining the surfaces of the trachea, bronchi, bronchioles and alveoli. Entry of the virus into target cells is facilitated by the viral spike protein. Angiotensin-converting enzyme 2 (ACE2) is the entry receptor used on the surface of the target cells, with the serine protease TMPRSS2 used for spike protein priming (Li et al. 2003; Nature. 426:450-454; Glowacka et al. 2011; J.Virol. 85:4122-4134).

The mechanisms by which the virus is able to trigger the plethora of symptoms exhibited remain under investigation. Central to this is triggering of the innate immune response. This process is multi-factorial with induction of mucus secretion, thereby initiating coughing. Likewise tissue resident granulocytes and macrophages interact with the virus, triggering a pro-inflammatory response and mobilization of the body’s immune cells and defense mechanisms. Pro-inflammatory cytokines, including interleukin (IL)-1, IL-6 and tumour necrosis factor alpha (TNF-α) to stimulate this response further. Such pathway activation results in a raise in body temperature by modulating the set-point in the hypothalamus.

Treatment options for COVID-19 are constantly evolving, with options including repurposed drugs such as dexamethasone, convalescent plasma, immunoglobulin based treatment, however there is a need for improved and efficacious treatment for COVID-infection and symptoms thereof..

MSC have been suggested for treatment of different neurological diseases such as including ALS and MS, and also for the treatment of Graft versus Host Disease (GvHD), arthritis, SLE, autoimmune Diabetes (Paladino et al., Stem Cells International Volume 2019, Article ID 3548917,) and a number of clinical studies have been conducted.

However, a limiting factor for the use of MSCs is the difficulty to obtain large batches of cells with desired properties. Furthermore, expansion of cells from one donor is generally used for production of a single batch, the next batch will typically use cells from another donor. Hence, the batch-to-batch variability is of major concern for both safety and efficacy. To overcome this issue, some researchers have developed methods for pooling donors and expanding the cells in vitro as a mixed donor product. These methods, while overcoming issues associated with cell numbers, also suffer from the same batch-to-batch heterogeneity issues, due to differential responses to pooling of donors, for example shifts in the expression of key immunosuppressive factors and enhancement of pro-inflammatory factors (WO 2016/193836, WO 2012/131618).

These challenges cause the cell therapy industry to go through cumbersome manufacturing processes with extensive testing and as consequence of excessive expansion, the cells might lose their potency and/or exhibit an increased risk for genetic instability. Additionally, when expansion of cells is done on a case to case basis, and this it is difficult to ensure optimal dosage of MSCs for the recipient patient and “giving the patient the number of cells we managed to expand” is a common approach. This makes treatment outcomes as well as potential adverse side effects highly unpredictable. Furthermore, the process of finding a suitable donor may be time consuming and laboursome, and carries an uncertainty regarding if a suitable donor will be found or not. Additionally, there is a risk that patients develop antibodies against the transplanted MSCs. In particular, a dose response relation between MSCs administered and the patient developing antibodies directed against the MSCs is expected. This effect can be profound by multiple administrations.

Thus, there is a large need in the field to provide a treatment and/or prevention of inflammatory diseases or conditions, autoimmune disease, transplantation rejection, CNS disorders and viral disorders, including but not limited to coronavirus such as COVID-19,ln particular, there is a need for a MSC population in this context, which enables administration of a suitable dosage of cells to a patient in need thereof. The production should ensure a robust manufacturing process with little variations between batches and every batch should yield multiple doses. The cells need to have proven potency and be formulated to minimize the risk of allosensitization and/or donor specific antibodies. It is furthermore desirable that said population is instantaneously available to a patient without the need for donor-recipient matching.

### SUMMARY OF THE DISCLOSURE

The object of the present disclosure is to provide methods, agents and treatments for COVID-19 infection or the treatment and/or prevention of symptoms associated with COVID-19 infection, which overcome the drawbacks of the prior art. It is envisioned that treatments with the isolated, pooled allogenic MSC population as described herein are an interesting therapeutic option.

Thus, the present disclosure aims at providing a treatment for COVID-19 infection or treatment and/or prevention of symptoms associated with COVID-19 infection of by using a MSC population suitable for transplantation (for example, but not limited to infusion or injection) to a patient in need thereof, which population comprises potent cells, exhibits low, or even no statistically significant, batch-to-batch variability and results in low alloimmunization or allosensitization in treated patients. The present object is achieved by the use of an isolated, pooled allogenic MSC population obtainable by the method disclosed herein, which employs the selection algorithm as described herein.

As used herein, the term “selection algorithm” refers to step 2-5 of the method defined below, in other words to all method steps disclosed except the culturing or providing step and the pooling step. It will be understood that further steps may be added to the selection algorithm without falling outside the scope of the present disclosure.

Within the context of COVID-19 the presence of a pro-inflammatory microenvironment, for example, elevated levels of TNF-α and interferon (IFN)-γ and/or stimulation or toll-like ligand receptor 3 by viral RNA, MSCs release prostaglandin E2 (PGE2), indoleamine 2,3 dioxygenase (IDO) and transforming growth factor beta 1 (TGFβ1), acting as anti-inflammatory signals that can promote regulatory T cell (Treg) and regulatory dendritic cell frequencies (Reviewed by Bernardo and Fibbe 2013; Cell Stem Cell. 13(4):392-402).

THUS, IN A FIRST ASPECT OF THE PRESENT DISCLOSURE, THERE IS PROVIDED AN ISOLATED, POOLED ALLOGENEIC MSC POPULATION FOR USE IN THE TREATMENT AND/OR PREVENTION OF COVID-19 INFECTION OR FOR USE IN THE TREATMENT AND/OR PREVENTION OF SYMPTOMS ASSOCIATED WITH COVID-19 INFECTION, WHEREIN SAID AN ISOLATED, POOLED ALLOGENEIC MSC POPULATION COMPRISES MSCS DERIVED FROM AT LEAST 3 INDIVIDUAL DONORS, WHEREIN THE NUMBER OF CELLS DERIVED FROM ANY ONE DONOR DOES NOT EXCEED 50% OF THE TOTAL CELL NUMBER AND WHEREIN SAID MSCS HAVE AT MOST BEEN SUBJECT TO TEN PASSAGES; AND WHEREIN SAID ISOLATED, POOLED ALLOGENEIC MSC POPULATION IS OBTAINABLE BY A METHOD comprising the steps of:
culturing or providing MSCs from more than said at least 3 individual donors to obtain more than at least 3 individual donor derived MSC populations;
assaying each individual donor derived MSC population using at least 3 assays to obtain at least 3 assay results for said each individual donor derived MSC population;
for each assay allocating an individual ranking score value to said each individual donor derived MSC population based on the assay result and thus obtaining at least 3 individual ranking score values for each individual donor derived MSC population, wherein a higher ranking score value is indicative of more desirable assay result; or wherein a lower ranking score value is indicative of more desirable assay result;
allocating a total score value to each individual donor derived MSC population based on said at least 3 individual ranking score values, wherein in the case of a higher ranking score value being indicative of more desirable assay result, a higher total score value is indicative of more desirable population properties; or wherein in the case of a lower ranking score value being indicative of more desirable assay result, a lower total score value is indicative of more desirable population properties;
selecting a subset of individual donor derived MSC populations with desirable population properties based on their total score values; and
pooling said selected individual donor derived MSC populations to obtain an isolated, pooled allogeneic MSC population;
wherein at least 2 of said at least 3 assays are selected from the group consisting of one assay measures indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs) and wherein at least 1 of said at least 3 assays is selected from the group consisting of one assay measuring the effect of said MCSs on the capacity of T cells to suppress an immune response; one assay measuring the effect said MCSs on the proliferation and/or apoptosis of dendritic cells; one assay measuring the effect of the said MSCs on monocytes and one assay measuring the effect of said MSCs on microglia cell and/or microglia-like cells.

The herein disclosed method for obtaining an isolated, pooled allogeneic MSC population for use as disclosed herein comprising cells from at least 3 individual donors, wherein the number of cells derived from any one donor does not exceed 50% of the total cell number, thus ensuring that the population comprises a significant number of cells derived from each donor and that cells derived from any one donor are not dominant in the population. It is considered beneficial that the population comprises similar numbers or numbers in the same range of cells derived from different individual donor. The present inventors expect that an isolated, pooled allogeneic MSC population obtained according to the method will exhibit low immunogenic properties. The selection algorithm is used herein to select cells with desired functionalities. Furthermore, the pooling of cells from multiple donors meeting the criteria of the selection algorithm will decrease batch-to-batch variability. The method also ensures that the isolated, pooled allogeneic MSC population comprises potent cells, as the selection algorithm functions to select cells with desirable properties. Additionally, the method as described herein allows for obtaining large batches of cells due to the pooling step. In particular, large batches of cells may be obtained, which cells have been subjected to a low number of passages. Furthermore, it should be highlighted that pooling of the product is restricted to the formulation step of obtaining the final drug product, thereby ensuring that no additional expansion of the cells, and the associated negative impact of said process on the potency and functionality of the product, is encountered. For example prior art documents WO 2016/193836, WO 2012/131618 teach that pooling and subsequent expansion of a cell product can result in a loss of immunosuppressive and/or immune-modulatory potential and an increase in inflammatory markers. Furthermore, said documents disclose that this effect is differential across pooled batches, therefore indicating a negative impact on batch-to-batch variation with donor mixing. Additionally, large batches also allow for reduction in manufacturing costs. In contrast, if cells are not pooled, it is difficult to obtain large batches of cells, especially if cells are subjected to a low number of passages. Surprisingly, data from the inventors demonstrate in fact that pooling of the product, without further expansion of the cells can lead to an enhanced immunosuppressive and/or immune-modulatory potential compared to the single donor cells of which the pooled product is comprised. Furthermore, a low passage number is associated with high potency in MSCs and it is therefore desirable that cells are not exposed to excessive numbers of passages. For clarity, a subculture is a new cell or microbiological culture made by transferring some or all cells from a previous culture to fresh growth medium. This action is called subculturing or passaging the cells. To record the approximate number of divisions cells have had in culture the number of passages may be recorded. As used herein, the term “passage” refers to transferring cells from a previous culture to fresh growth medium.

Thus, in one embodiment there is provided an isolated, pooled allogeneic MSC population for use as disclosed herein, wherein said MSC in the isolated, pooled allogeneic MSC population have at most been subject to ten passages, such as most nine passages, such as most eight passages, such as most seven passages, such as at most six passages, such as at most five passages, such as at most four passages, such as at most three passages, such as one, two or three passages, such as two or three passages. It is to be appreciated that the number of passages is related to the number of cells present in the culture. Thus, it may be beneficial to retain a balance between cell number and maintained potency in order to obtain a sufficient number of cells with desirable properties. Thus, in some embodiments the said MSC have been subject to from 2 to 6, such as from 2 to 5, such as from 2 to 4, such as from 2 to 3 passages.

Mesenchymal stem cells (MSCs) are non-hematopoietic cells expressing the surface markers CD73, CD90, and CD105 while lacking the expression of CD14, CD34, and CD45 or CD11b, CD79alpha or CD19 and HLA-DR surface molecules. In vitro, MSCs adhere to plastic under standard tissue culture conditions, and have the capacity to differentiate into osteoblasts, adipocytes and chondroblasts. As used herein, the terms “MSCs”, “mesenchymal stem cells”, “mesenchymal stromal cells” and “marrow stromal cells” refer to cells with the above-mentioned properties. The present disclosure adheres to the definition of MSC according to the criteria of the International Society for Cell and Gene Therapy (ISCT). MSCs can be derived from tissues including bone marrow, peripheral blood, adipose tissue, dental tissue, placenta, umbilical cord, amniotic fluid, cord blood, Wharton Jelly, decidua, chondrion membrane and amnion membrane. Without being bound be theory, MSCs are considered to be well suited to treat the complex diseases, such as inflammatory diseases or conditions, autoimmune disease, transplantation rejection, and CNS disorders (in particular of CNS disorders) because of their wide range of potential therapeutic responses, including direct cell replacement, trophic factor delivery, and immunomodulation. Some investigators in preclinical studies have given insight into possible MSC mechanisms in treating CNS disorders, such as ALS. Their most important mechanism of action of the treatment of ALS is most likely the creation of a protective milieu near the motor neurons through secretion of neuroprotective factors, reduction of neuroinflammation and inhibition of motor neuron apoptosis. Potential mechanisms of mesenchymal stem cell efficacy in neurodegeneration may be achieved through paracrine effects and cell-to-cell contacts with resident neural cells. The capacity of MSCs to secrete cytokines, growth factors and exosomes could potentially induce and support regeneration processes, including angiogenesis, synaptogenesis, axonal re-myelination and neurogenesis. Because of their immunomodulatory properties, MSCs could attenuate inflammatory responses in the central nervous system by inhibiting maturation and migration of dendritic cells, suppression of lymphocyte activation and proliferation and by reducing gliosis. Moreover, MSCs possess anti-apoptotic properties, and may limit excitotoxicity by modulating astrocyte function. Additionally, compared to other types of stem cell (embryonic stem cells or induced pluripotent stem cells), MSCs have a better biosafety profile and lower risk of tumourgenicity (Ra et al., (2011). Stem Cells Dev, 20, 1297-308).

Thus, in one embodiment, there is provided an isolated, pooled allogeneic MSC population for use disclosed herein, wherein said MSCs are selected from the group consisting of bone marrow derived MSCs, peripheral blood derived MSCs, adipose tissue derived MSCs, dental tissue derived MSCs, oral mucosa derived MSCs, placenta derived MSCs, umbilical cord derived MSCs, amniotic fluid derived MSC, cord blood derived MSCs, Wharton Jelly derived MSCs, decidua derived MSCs, chondrion membrane derived MSCs and amnion membrane derived MSCs. In particular embodiments, said MSCs are selected from the group consisting of placenta derived MSCs, umbilical cord derived MSCs, amniotic fluid derived MSC, oral mucosa derived MSCs, cord blood derived MSCs, Wharton Jelly derived MSCs, decidua derived MSCs, chondroid membrane derived MSCs, dental pulp and amnion membrane derived MSCs; such as placenta derived MSCs, umbilical cord derived MSCs, amniotic fluid derived MSC, cord blood derived MSCs, Wharton Jelly derived MSCs, decidua derived MSCs, dental pulp derived MSCs and amnion membrane derived MSCs; such as placenta derived MSCs, umbilical cord derived MSCs, amniotic fluid derived MSC, cord blood derived MSCs, Wharton Jelly derived MSCs, dental pulp derived MSCs; such as placenta derived MSCs, umbilical cord derived MSCs, cord blood derived MSCs and Wharton Jelly derived MSCs; such as umbilical cord derived MSCs, cord blood derived MSC and Wharton Jelly derived MSCs.

It will be appreciated that MSC or cells exhibiting MSC characteristics which cells have been transdifferentiated or dedifferentiated into MSCs carry epigenetic characteristics of their previous fate (also referred to as epigenetic memory), which may affect the properties of said transdifferentiated or dedifferentiated MSC. Without being bound by theory, for example, a population of said cells may express MSC markers to a lower degree than a population of native MSCs and/or may affect other cell populations to a lesser extent compared to native MSCs. In contrast, MSCs derived from a native MSC source (in other words native MSCs), for example from any one of the cell sources listed above including but not limited to Wharton’s Jelly, have not been manipulated into a cell outside of their germ layer and thus there is no negative impact on factors such as marker expression or functionality. It is considered that said MSCs derived from a native MSC source therefore may exhibit a higher degree of desirable properties.

Thus, in one embodiment of the present aspect, said MSCs are derived from a native MSC source.

As used herein, the term “derived from” in reference to a source of MSCs is to be understood to mean the same as “isolated from”. These terms are used interchangeably in the present disclosure.

As used herein, the term “native MSC source” refers to a source of MSC which is present within fetal and adult organs and isolating or deriving MSC therefrom does not require any manipulation of the cells to induce a characteristic MSC phenotype. It is assumed that someone skilled in the art would appreciate that this phenotype would be defined as per the ISCT guidelines for expanded MSC sources, and that primary MSCs express a different cell surface marker profile prior to contact with plastic and expansion. Also isolating or deriving MSC from a native source does not require any transdifferentiation and dedifferentiation step.

As used herein, the term “transdifferentiation” is used to describe the process by which one mature cell type transitions to another mature type with a different function and/or phenotype. This process can occur artificially, for example lineage reprogramming or in response to environmental cues both in vivo and ex vivo.

As used herein, the term “dedifferentiation” refers to a process whereby cells regress from a specialized function to a simpler state reminiscent of stem or progenitor cells.

In recent years, MSCs have emerged as a potential candidate in cell therapy of neurodegenerative diseases due to their multi-facet functions in tissue regeneration. Particularly, the immune-modulatory properties of MSCs have been identified to play an important role in their therapy for inflammatory diseases including neurodegenerative disorders. Additionally, studies have indicated that umbilical cord derived MSCs or Wharton Jelly derived MSCs are nontumourigenic, anti-tumorigenic, and do not transform to the TAF phenotype that is associated with enhanced growth of solid tumours, and suppress hematopoietic tumour development. Therefore, umbilical cord derived MSCs or Wharton Jelly derived MSCs (also referred to herein as WJMSCs) may be particularly useful in this context. Thus, in one embodiment, said MSCs are umbilical cord derived MSCs or Wharton Jelly derived MSCs, such as Wharton Jelly derived MSCs.

WJMSCs have been shown to have high immunomodulatory capabilities, as well as good proliferation and differentiation potential and are readily available as a cell source; therefore, WJMSCs may be an important cell therapy source. WJMSCs are known to have immunoprivileged characteristics and are less immunogenic than BM-MSC as well as foetal MSCs which may be an advantage in an allogeneic setting.

Preclinical studies have shown that MSCs express 12 neural genes and 11 transcription factors (Blondheim, 2006, Stem Cells Dev. Apr;15(2):141-64.). Compared to BM-MSCs, WJ-MSCs have been shown to overexpress genes involved in neurotrophic support, neuronal maturation (Drela et al, 2016 Cytotherapy. Apr;18(4):497-509, cell adhesion, proliferation, and immune system function and under adequate stimulation WJMSCs can differentiate into neuron-like cells in vitro (Donders, 2018, Stem Cells Dev. Jan 15;27(2):65-84; Ishii, Neurosci Lett;163:159-62). Thus, without being bound by theory, WJMSCs may be a suitable for cell therapy of CNS disorders, including neurodegenerative disorders.

In some embodiments of the isolated, pooled allogeneic MSC population for use as disclosed herein, it may be beneficial that the isolated, pooled allogeneic MSC population comprises MSCs derived from more than 3 donors in order to ensure that the concentration of any allogenic Human Leukocyte Antigen (HLA) will be lower than when cells from a single donor or from few donors were used. It is envisioned that this will reduce the risk of generation of anti-HLA antibodies (i.e. Donor specific antibodies, DSA) in patients administered the isolated, pooled allogeneic MSC population. The present inventors expect that low concentration of any specific HLA allele in the isolated, pooled allogeneic MSC population will reduce the risk of adverse effect connected to single and multiple administrations of said cells. Additionally, by using cells from multiple donors, low batch variability can be obtained. The donor to donor variability between donors that have qualified for manufacturing and that have passed all GMP quality criteria in the expansion to large scale clinical grade drug product is reduced by up to 40 % or even more when comparing the results from all donors with the results from the donors selected for pooling. It is envisioned that the reduction in variation for specific assays generates an overall assessment reduction of variation, based on the selection algorithm, of up to 40 % or oven more between selected donors and all donors evaluated for a specific batch. The GMP production of MSC will dramatically reduce the donor variability and the Selection algorithm will further reduce the variation by up to 40 % or even more, resulting in batch-to-batch variation without statistical significance.

Thus, in one embodiment, said population for use as disclosed herein comprises MSCs derived from at least four individual donors, such as at least five individual donors, such as at least six individual donors, such as at least seven individual donors, such as at least eight individual donors, such as at nine individual donors, such as at least ten individual donors. In another embodiment, the isolated, pooled allogeneic MSC population for use comprises MSCs derived from 3-20 individual donors, such as 3-15 individual donors, such as 3-10 individual donors, such as 4-8 individual donors, such as 5-7 individual donors, such as 5, 6 or 7 individual donors. In one particular embodiment, said step of assaying each individual donor derived MSC population comprises assaying at least one more, such as at least two more, such as at least three more, such as at least four more, such as at least five more, such as at least six more, such as at least seven more, such as least eight more, such as at least nine more, such as at least ten more individual donor derived MSC population than the number of individual donor derived MSC populations pooled in the pooling step. In one particular embodiment, said the step of assaying each individual donor derived MSC population comprises assaying at least 1-4 times, such as 2-4 times, such as 2-3 or 3-4 times, as many individual donor derived MSC population as the number of individual donor derived MSC populations pooled in the pooling step. Thus, for example, if the isolated, pooled allogeneic MSC population for use as disclosed herein comprises MSC derived from 3 individual donor derived MSC populations, the step of assaying each individual donor derived MSC population comprises assaying 3-12, such as 6-12, such as 6-9 or 9-12 individual donor derived MSC populations. In this example, only 3 individual donor derived MSC populations would be selected for pooling, while the remaining individual donor derived MSC populations would be discarded.

In one embodiment of the disclosure, said step of culturing or providing MSCs comprises culturing or providing MSCs from at least 4 individual donors to obtain said at least 4 individual donor derived MSC populations, such as at least 5, such as at least 6, such as at least 7, such as at least 8, such as at least 9, such as at least 10, such as at least 11, such as at least 12, such as at least 13, such as at least 14, such as at least 15, such as at least 16, such as at least 17, such as at least 18, such as at least 19, such as at least 20 individual donor derived MSC populations. For example, from about 3 to about 50 individual donor derived MSC populations, such as from about 4 to about 50, such as from about 5 to about 50, such as from about 6 to about 50, such as from about 6 to about 30, such as from about 6 to about 20, such as from about 6 to about 15, such as from about 8 to about 12 individual donor derived MSC population may be provided or cultured.

In some embodiments of the isolated, pooled allogeneic MSC population for use as disclosed herein, the step of assaying said each individual donor derived MSC population, comprises assaying at least 3, such as at least 4, such as at least 5, such as at least 6, such as at least 7, such as at least 8, such as at least 9, such as at least 10, such as at least 11, such as at least 12, such as at least 13, such as at least 14, such as at least 15, such as at least 16, such as at least 17, such as at least 18, such as at least 19, such as at least 20 individual donor derived MSC populations, In another embodiment, the step of assaying said each individual donor derived MSC population comprises assaying from about 3 to about 50 individual donor derived MSC populations, such as from about 4 to about 50, such as from about 5 to about 50, such as from about 6 to about 50,such as from about 6 to about 30, such as from about 6 to about 20, such as from about 6 to about 15, such as from about 8 to about 12 individual donor derived MSC populations. In one embodiment, from about 3 to about 20 individual donor derived MSC populations are assayed, for example, 8, 9, 10, 11, 12, 13, 14 individual donor derived MSC population may be assayed. It is to be understood that the individual donor derived MSC populations assayed in the present step of the method as described herein, are obtained in the culture or provision step according to the said method.

It will be appreciated that the immunosuppressive capacity of MSCs as disclosed herein may be of large importance for their suitability for therapeutic uses. As used herein, the term “immunosuppressive capacity” refers to the capacity to elicit a reduction of the activation or efficacy or a modulation of the function of the immune system. The skilled person will appreciate that the immunosuppressive capacity may be measured directly or indirectly in an assay.

The isolated, pooled allogeneic MSC population for use as described herein is obtainable by a method that comprises a step of assaying each individual donor derived MSC population using at least 3 assays to obtain at least 3 assay results for said each individual donor derived MSC population. As disclosed herein, 2 of said at least 3 assays are selected from the group consisting of one assay measures indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs).

An immunosuppressive potential may reported as a measure of IDO activity, determined by measuring tryptophan and kynurenine in the culture supernatant. IDO is a heme-containing enzyme that in humans is encoded by the IDO1 gene. The IDO enzyme converts L-tryptophan to N-formylkynurenine (or kynurenine), an immunosuppressive molecule that acts as an inhibitor of immune cell proliferation, including T cells. The IDO activity may be presented as the ratio of kynurenine/tryptophan and can be determined by calculating the amount of tryptophan and kynurenine present in cell culture supernatants for example using an ELISA kit. When stimulated with interferon gamma (IFNγ), in the presence or absence of tumor necrosis factor, mesenchymal stem/stromal cells (MSCs) secrete more IDO than when they are unstimulated. In one embodiment, said assay measuring IDO activity comprises or consists of the step of measuring IDO activity within the culture supernatant of MSCs co-cultured with stimulated PBMCs or purified T cells or activated monocytes/macrophages or microglia. In one embodiment, measuring IDO activity may be performed as described above. Inducible IDO activity indicates that the cells have functional potency, related to antibacterial and antiviral function, immunomodulation and/or immunosuppression which the present inventors consider a key quality attribute of the MSCs described herein. Said assay measuring indoleamine-2,3-dioxygensase (IDO) activity thus immunosuppressive capacity of said MSCs.

Furthermore, the MSCs may be assayed to measure prostaglandin E2 secreted by said MSCs. Prostaglandin E2 (PGE2) is formed in a variety of cells from prostaglandin H2, which is synthesized from arachidonic acid by the enzyme prostaglandin synthetase. PGE2 has a number of biological actions, including vasodilation, both anti- and proinflammatory action, modulation of sleep/wake cycles, and facilitation of human immunodeficiency virus replication. PGE2 is active in inflammation, immune regulation, generation of fever, pain perception, protection of the gastric muscosa, fertility and parturition, as well as sodium and water retention. Likewise, PGE2 has antifibrotic functions. PGE2 is rapidly metabolized in vivo, the half-life of PGE2 in the circulatory system is approximately 30 seconds and normal plasma levels are 3-12 pg/mL. PGE2 is involved in the regulation of different stages of the immune response and different effector mechanisms of immunity. MSCs constitutively produce PGE2, and their proliferation is regulated by this prostaglandin through the differential activation of cAMP-dependent protein kinase isoforms. This production of PGE2 is sensitive to the local environment, where inflammatory signals stimulate its induction. During co-culture with immune cells, and/or tumor necrosis factor alpha (both in combination with INFγ or alone), PGE2 production by MSCs is substantially increased and participates in the immunomodulatory effects of MSCs. Moreover, the role of PGE2 in MSC-induced immunosuppressive effects depends on T-cell stimuli, as reported by Rasmusson et al. (Rasmusson et al., (2005) Exp.Cell.Res, 305 (1) (2005), pp. 33-41). PGE2 is effective in the MSC inhibition of T cells activated by PHA rather than by alloantigens. MSCs prevent lymphocyte activation and induce the inhibition of T-cell proliferation through the modulation of COX1/ COX2 expression and ultimately PGE2 production. Therefore, it is possible use the amount of PGE2 secretion found in cell culture supernatants from co-cultures of peripheral blood mononuclear cells (PBMCs) and MSCs as a measure of immunosuppressive capacity. In one embodiment, said at least one assay measuring the immunosuppressive capacity of said MSCs measures prostaglandin E2 secreted by said MSCs. In one embodiment, said at least one assay measuring prostaglandin E2 secreted by said MSCs comprises measuring prostaglandin E2 secreted by said MSCs when co-cultured with PBMCs, such as PHA stimulated PBMCs, such as PHA stimulated T-lymphocytes, activated monocytes/macrophages and/or microglia. In one embodiment, said one assay measuring prostaglandin E2 secreted by said MSC comprises or consists of the step of measuring PGE2 secretion by MSCs co-cultured with INFγ and/or tumor necrosis factor alpha.

It is also possible to quantitatively measure the immunosuppressing effect the MSCs have on the proliferation of peripheral blood mononuclear cells (PBMC). MSCs have been shown to suppress T-lymphocyte proliferation. Mixed lymphocyte reactions with MSC are frequently used to demonstrate the immunosuppressive activity of MSC. In one embodiment, said least one assay measuring the immunosuppressive capacity of said MSCs measures the effect of said MSCs on the proliferation of peripheral blood mononuclear cells (PBMCs), such as T-lymphocytes. For example, the proliferation of T-lymphocytes, such as proliferation of phytohemagglutinin (PHA) stimulated T-lymphocytes. PHA is used as a mitogen which activates proliferation of T-lymphocytes. Thus, in one embodiment, said proliferation of PBMCs is the proliferation of T-lymphocytes, such as proliferation of PHA stimulated T-lymphocytes. The immunosuppressive activity of MSCs may be quantified as the decrease in proliferation of PHA stimulated T-lymphocytes.

It will be appreciated that said at least 2 of said at least 3 assay may be independently selected from the group consisting of one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs). Thus said at least two assays may be one assay measuring IDO activity and one assay measuring PGE2; or one assay measuring IDO activity and one assay measuring proliferation of PBMCs; or one assay measuring PGE2 and one assay measuring proliferation of PBMCs. Said at least 2 assays may also include all said three assays.

Similarly, it will be appreciated that said at least 1 of said at least 3 assay may be independently selected from the group consisting of one measuring the effect of said MCSs on the capacity of T cells to suppress an immune response; one assay measuring the effect said MCSs on the proliferation and/or apoptosis of dendritic cells, one assay measuring the effect of the said MSCs on monocytes and one assay measuring the effect of the said MSCs on microglia cell and/or microglia-like cells. However, said at least 1 assay may include any 2, or 3 or all 4 of said assays. Thus said at least 1 assay may be one assay measuring the effect of said MCSs on the capacity of T cells to suppress an immune response and one assay measuring the effect said MCSs on the proliferation and/or apoptosis of dendritic cells; or one assay measuring the effect said MCSs on the proliferation and/or apoptosis of dendritic cells and one assay measuring the effect of the said MSCs on microglia cell and/or microglia-like cells ; or one assay measuring the effect of said MCSs on the capacity of T cells to suppress an immune response and one assay measuring the effect of the said MSCs on microglia cell and/or microglia-like cells; or one assay measuring the effect of said MCSs on the capacity of T cells to suppress an immune response and one assay measuring the effect of the said MSCs on monocytes; or one assay measuring the effect said MCSs on the proliferation and/or apoptosis of dendritic cells and one assay measuring the effect of the said MSCs on monocytes; or one assay measuring the effect of the said MSCs on microglia cell and/or microglia-like cell and one assay measuring the effect of the said MSCs on monocytes. In one embodiment, said at least 1 assay may be one assay any 3 assays selected from selected from the group consisting of one measuring the effect of said MCSs on the capacity of T cells to suppress an immune response; one assay measuring the effect said MCSs on the proliferation and/or apoptosis of dendritic cells, one assay measuring the effect of the said MSCs on monocytes and one assay measuring the effect of the said MSCs on microglia cell and/or microglia-like cells. Also, said at least 1 assay may also include all said four assays.

To clarify, any at least 2 of said at least 3 assay may be independently selected from the group consisting of one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs) may be combined with any at least 1 of said at least 3 assay may be independently selected from the group consisting of one assay measuring the effect of said MSCs on the capacity of T cells to suppress an immune response; one assay measuring the effect said MSCs on the proliferation and/or apoptosis of dendritic cells, one assay measuring the effect of the said MSCs on monocytes and one assay measuring the effect of the said MSCs on microglia cell and/or microglia-like cells .

T regulatory (Treg) cells are identified as a subpopulation of the CD4+CD25+ T cell population with the capacity to suppress an immune response. This subpopulation may be further characterized by lack of expression of CD127 or positive expression of FoxP3. This fraction of cells is expected to increase when T cells are exposed said MSCs. This effect may for example be analyzed by flow cytometry. Thus, in one embodiment, said at least 1 of said at least 3 assays is an assay measuring the effect of said MSCs on the capacity of T cells to suppress an immune response. In one embodiment, said capacity of T cells to suppress an immune response is measured as the fraction of T regulatory cells, such as a fraction of CD25+ T cells, of a T cell population. For example, a fraction of CD4+CD25+ T cells of the total CD4+ T cell population. In one embodiment, effect is measured during coculture of said MSCs and T cells. In one embodiment, said coculture is in the presence of a stimulus, such as a stimulus selected from PHA and lipopolysaccharide (LPS). In one embodiment, an increase of the fraction of Treg expressing is indicative of a desirable result.

Fms-related tyrosine kinase 3-ligand (FLT3L) is a key regulator of dendritic cell (DC) commitment in hematopoiesis, which regulates the proliferation, differentiation and apoptosis of hematopoietic cells through the binding to FLT3 (Yuan et al (2019), Nature Communications volume 10, Article number: 2498). MSCs express FLT3L that binds to FLT3 on CD1c+DCs to promote the proliferation and inhibit the apoptosis of tolerogenic CD1c+DCs. MSC expression of FLT3L may be measured by ELISA in co-culture with PBMC, with or without stimulation with e.g. PHA or LPS.

The fraction of cells being CD1c+ is expected to increase in the presence of said MSCs as said MSC induce tolerance. This effect may for example be analyzed by flow cytometry.

Thus, in one embodiment, said at least 1 of said at least 3 assay is an assay measuring the effect said MCSs on the proliferation and/or apoptosis of dendritic cells. In one embodiment, said effect is measured during coculture of said MSCs and DC. In one embodiment, said coculture is in the presence of a stimulus, such as a stimulus selected from PHA and lipopolysaccharide (LPS). In one embodiment, an increase of the fraction of DCs expressing CD1c is indicative of a desirable result.

For example, in some embodiments of the isolated, pooled allogeneic MSC population for use as disclosed herein,, said at least three assays comprise one assay measuring indoleamine-2,3-dioxygensase (IDO) activity; one assay measuring prostaglandin E2 secreted by said MSCs; and one assay measuring the effect of said MCSs on the capacity of T cells to suppress an immune response or one assay measuring indoleamine-2,3-dioxygensase (IDO) activity; one assay measuring the effect of said MSCs on the proliferation of PBMCs; and one assay measuring the effect of said MCSs on the capacity of T cells to suppress an immune response,
or one assay measuring prostaglandin E2 secreted by said MSCs; one assay measuring the effect of said MSCs on the proliferation of PBMCs; and one assay measuring the effect of said MCSs on the capacity of T cells to suppress an immune response,
or one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs); and one assay measuring the effect of said MCSs on the capacity of T cells to suppress an immune response.

For example, in some embodiments as disclosed herein, said at least three assays comprise

one assay measuring indoleamine-2,3-dioxygensase (IDO) activity; one assay measuring prostaglandin E2 secreted by said MSCs; and one assay measuring the effect said MCSs on the proliferation and/or apoptosis of dendritic cells, or one assay measuring indoleamine-2,3-dioxygensase (IDO) activity; one assay measuring the effect of said MSCs on the proliferation of PBMCs; and one assay measuring the effect said MCSs on the proliferation and/or apoptosis of dendritic cells, or one assay measuring prostaglandin E2 secreted by said MSCs; one assay measuring the effect of said MSCs on the proliferation of PBMCs; and one assay measuring the effect said MCSs on the proliferation and/or apoptosis of dendritic cells or one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs); and one assay measuring the effect said MCSs on the proliferation and/or apoptosis of dendritic cells.

It will be understood that the method for obtaining the isolated, pooled allogeneic MSC population for use as disclosed herein may also comprise further assays.

Microglia are a type of neuroglia (glial cell) located throughout the brain and spinal cord. Microglia account for 10-15% of all cells found within the brain and as they act as the first and main form of active immune defense in the central nervous system (CNS). Upon activation, microglia are capable of acquiring diverse phenotypes that display different cell surface and intracellular markers, secrete different factors, and exhibit different functions. Furthermore, the cells are capable of shifting between the different phenotypes, for example M1 to M2 phenotype, during an inflammatory response. M1 microglia are typically the initial responders to an insult. Cytokines released by astrocytes and Th1 cells, including IFNγ and TNF-alpha (tumor necrosis factor alpha), bacterial-derived products, such as lipopolysaccharide (LPS), and trauma-induced cellular debris will polarize microglia toward the M1 phenotype. M1 microglia will produce proinflammatory cytokines, chemokines, and redox signalling molecules. They will also express scavenger receptors, and MHC class II and co-stimulatory molecules on their cell surface. These actions allow M1 microglia to kill and phagocytize foreign and cellular debris, and recruit and differentiate T cells in order to launch an immune response. Over time, the inflammatory response is shifted to be more anti-inflammatory, which is facilitated by M2 microglia. Microglia are polarized to the M2 phenotype following stimulation with IL-4 or IL-13, which are typically released from Th2 cells. M2 microglia secrete anti-inflammatory cytokines and growth factors that promote attenuation of the inflammatory response and repair of damaged tissue.

One or several assay(s) may be used to, for example quantitatively or qualitatively, measure the immunosuppressing effect that said MSCs have on the proliferation of microglia cells or assay the effect of said MCSs on microglia phenotype. As used herein, these assays are referred to as “microglia assays”. Said microglia assays may use immortalized cell lines, such as for example HMC3 cells or CHME5 cells. Alternatively, primary microglia from biopsies may be used or primary microglia-like cells cultured from cord blood, or immortalized microglia-like cells for example DUOC-01 cells. The skilled person is familiar with other cell lines (immortalized or primary) which may be suitable for use in microglia assays.

In one embodiment of the isolated, pooled allogeneic MSC population for use as disclosed herein, said one assay measuring the effect of the said MSCs on microglia cell or microglia-like cells is selected from the group consisting of one assay measuring microglia cell or microglia-like cell proliferation; one assay measuring expression of markers characteristic of the M1 phenotype in microglia cells or microglia-like cells; one assay measuring expression of markers characteristic of the M2 phenotype in microglia cells or microglia-like cells; and an assay measuring the shift from the M1 microglia phenotype to the M2 microglia phenotype in microglia cells or microglia-like cells.

MSC have been shown to suppress microglia proliferation. Co-culture of microglia and MSC may be used to demonstrate the immunosuppressive activity of MSC. Lipopolysaccharides (LPS) may be used as a mitogen which activates proliferation of microglia. The immunosuppressing effect of said MSCs may be quantified as the decrease in proliferation of mitogen stimulated, such as LPS stimulated, microglia cells or microglia-like cells.

In one embodiment, said one assay measuring microglial proliferation comprises cocultivation of said individual donor derived MSC population(s) with microglia cells and/or microglia-like cells. It will be understood that an assay measuring the immunosuppressing effect of said MSCs on microglia or microglia-like cells may be performed in conditions of cocultivation, but may also be performed in a transwell cell culture setup or using conditioned media from MCS cultivation. The skilled person is aware of different variants and experimental setups that may be used.

In one embodiment, said microglia cells or microglia-like cells are selected from the group consisting of immortalized cell lines, such as the human microglial HMC3 cell line or the CHME-5 cell line; primary microglia obtained from biopsies; primary microglia-like cells cultured from cord blood; and immortalized microglia-like cells from cord blood, such as the DUOC-01 cell line. In one embodiment, said microglia cells or microglia-like cells are selected from immortalized cell lines. In one embodiment, said microglia cells or microglia-like cells are selected from the group consisting of immortalized cell lines are selected from the group consisting of the HMC3 cell line, CHME-5 cell line and the DUOC-01 cell line.

In one embodiment, said one assay measuring microglial proliferation comprises assaying if a decrease in the proliferation microglia cells or microglia-like cells occurs upon mitogen, such as lipopolysaccharide, stimulation or quantifying a decrease in the proliferation microglia cells or microglia-like cells upon mitogen, such as lipopolysaccharide, stimulation. Said proliferation may be measured as a proliferation percentage, may be measured as a proliferation index, may be measured by counting cells or may be measured as a growth index, such as may be measured as a growth index.

Microglia and/or microglia-like cells of M1 phenotype are characterized by expression of one or more of the following markers CD183, CD11b, CD14, B7-2/CD86, Integrin alpha V beta 3, MFG-E8, NO, ROS, RNS, CCL2/MCP-1, CCL3/MIP-1 alpha, CCL4/MIP-1 beta, CCL5/RANTES, CCL8/MCP-2, CCL11/Eotaxin, CCL12/MCP-5, CCL15/MIP-1 delta, CCL19/MIP-3 beta, CCL20/MIP-3 alpha, CXCL1/GRO alpha/KC/CINC-1, CXCL9/MIG, CXCL10/IP-10, CXCL11/I-TAC, CXCL13/BLC/BCA-1, CX3CL1/Fractalkin, MMP-3, MMP-9, Glutamate, IL-1 beta/IL-1F2, IL-2, IL-6, IL-12, IL-15, IL-17/IL-17A, IL-18/IL-1F4, IL-23, IFNγ, TNF-alpha, Fc gamma RIII/CD16, Fc gamma RII/CD32, CD36/SR-B3, CD40, CD68/SR-D1, B7-1/CD80, MHC II, iNOS and COX-2. Microglia and/or microglia-like cells of M2 phenotype are characterized by expression of one or more of the following markers CX3CR1, CD200R, CD206, IL-1Ra/IL-1F3, IL-4, IL-10, IL-13, TGF-beta, CCL13/MCP-4, CCL14, CCL17/TARC, CCL18/PARC, CCL22/MDC, CCL23/MPIF-1, CCL24/Eotaxin-2/MPIF-2, CCL26/Eotaxin-3, FIZZ1/RELM alpha, YM1/Chitinase 3-like 3, CLEC10A/CD301, MMR/CD206, SR-AI/MSR, CD163, Arginase 1/ARG1, Transglutaminase 2/TGM2, PPAR and gamma/NR1C3.

By measuring the expression of any one or more of said markers, the phenotype characteristics of the microglia and/or microglia-like cells can be determined. CX3CR1 (Fractalkine receptor) is upregulated on microglia with M2 phenotype (desired). The MSC should also have increased expression of CX3CL1 (Fractalkine ligand). The ligand is cleaved by metalloproteinase and binds to the receptor. Thus, media concentration of fractalkine ligand should be low to reflect an active M2 phenotype. CD200R is upregulated on microglia with M2 phenotype, which is desirable in the present context. The MSC should also have increased expression of CD200 (which is the ligand that binds to CD200R).

The expression may be analyzed by any method known to the person skilled in the art, including but not limited to flow cytometry, antibody staining, in situ-hybridization.

Thus, in one embodiment, said one assay measuring expression of markers characteristic of the M1 phenotype in microglia and/or microglia-like cells comprises measuring the expression of at least one marker selected from the group consisting of CD183, CD11b, CD14, B7-2/CD86, Integrin alpha V beta 3, MFG-E8, NO, ROS, RNS, CCL2/MCP-1, CCL3/MIP-1 alpha, CCL4/MIP-1 beta, CCL5/RANTES, CCL8/MCP-2, CCL11/Eotaxin, CCL12/MCP-5, CCL15/MIP-1 delta, CCL19/MIP-3 beta, CCL20/MIP-3 alpha, CXCL1/GRO alpha/KC/CINC-1, CXCL9/MIG, CXCL10/IP-10, CXCL11/I-TAC, CXCL13/BLC/BCA-1, CX3CL1/Fractalkine, MMP-3, MMP-9, Glutamate, IL-1 beta/IL-1F2, IL-2, IL-6, IL-12, IL-15, IL-17/IL-17A, IL-18/IL-1F4, IL-23, IFNγ, TNF-alpha, Fc gamma RIII/CD16, Fc gamma RII/CD32, CD36/SR-B3, CD40, CD68/SR-D1, B7-⅟CD80, MHC II, iNOS and COX-2; such as at least one marker selected from the group consisting of CD183, CD11b, CD14, B7-2/CD86, CD40 and B7-1/CD80; such as at least one marker selected from the group consisting of CD183, CD11b and CD14. In particular, in one embodiment said one assay measuring expression of markers characteristic of the M1 phenotype in microglia and/or microglia-like cells comprises measuring the expression of at least CD183.

In one embodiment, a decrease in expression of at least one of the markers whose expression in measured by said one assay measuring expression of markers characteristic of the M1 phenotype in microglia and/or microglia-like cells is indicative of a desirable result.

In one embodiment, said one assay measuring expression of markers characteristic of the M2 phenotype in microglia and/or microglia-like cells comprises measuring the expression of at least one marker selected from the group consisting of CX3CR1, CD200R, CD206, IL-1ra/IL-1F3, IL-4, IL-10, IL-13, TGF-beta, CCL13/MCP-4, CCL14, CCL17/TARC, CCL18/PARC, CCL22/MDC, CCL23/MPIF-1, CCL24/Eotaxin-2/MPIF-2, CCL26/Eotaxin-3, FIZZ1/RELM alpha, YM1/Chitinase 3-like 3, CLEC10A/CD301, MMR/CD206, SR-AI/MSR, CD163, Arginase 1/ARG1, Transglutaminase 2/TGM2, PPAR and gamma/NR1C3; such as at least one marker selected from the group consisting of CX3CR1/Fractalkine Receptor, CD200R, CD206 and CD163; such as at least one marker selected from the group consisting of CX3CR1, CD200R and CD206.

In particular, in one embodiment said one assay measuring expression of markers characteristic of the M2 phenotype in microglia and/or microglia-like cells comprises measuring the expression of at least CD200R.

In one embodiment, an increase in expression of at least one of the markers whose expression in measured by said one assay measuring expression of markers characteristic of the M2 phenotype in microglia and/or microglia-like cells is indicative of a desirable result.

Additionally, a shift from M1 phenotype to M2 phenotype may be measured by a change in expression of any one of more of said markers. For example, as shift from M1 phenotype to the M2 phenotype of said microglia and/or microglia-like cells is associated with the decrease in expression levels of any one or more of the M1 markers and with the increase in expression levels of any one or more of the M2 markers.

Thus, in one embodiment, wherein said shift from the M1 microglia and/or microglia-like cell phenotype to the M2 microglia and/or microglia-like cell phenotype is measured as a decrease in the expression of any one or more of the M1 markers defined above and an increase in the expression of any one or more of the M2 markers defined above. In particular, said shift from the M1 microglia and/or microglia-like cell phenotype to the M2 microglia and/or microglia-like cell phenotype is measured as a decrease in the expression of any one or more of the markers selected from CD183, CD11b, CD14, B7-2/CD86, CD40 and B7-1/CD80, and an increase in the expression of any one or more of the markers selected from CX3CR1/fractalkine receptor, CD200R, CD206 and CD163; such as wherein said shift from the M1 microglia and/or microglia-like cell phenotype to the M2 microglia and/or microglia-like cell phenotype is measured as a decrease in the expression of any one or more of the markers selected from CD183, CD11b and CD14 and an increase in the expression of any one or more of the markers selected from CX3CR1, CD200R and CD206, such as wherein said shift from the M1 microglia and/or microglia-like cell phenotype to the M2 microglia and/or microglia-like cell phenotype is measured as a decrease in the expression of CD183 and an increase in the expression of CD200R. In one embodiment, said shift from the M1 microglia and/or microglia-like cell phenotype to the M2 microglia and/or microglia-like cell phenotype is indicative of a desirable result, such as an induction of an anti-inflammatory effect in said microglia and/or microglia-like cells. For example, a shift score may be calculated according to the following generalized formula:

$$S h i f t s c o r e = \frac{M 2 f o l d i n c r e a s e}{1 - M 1 s u p p r e s s i o n} \qquad \text{(formula 1)}$$

Thus, in one embodiment of the isolated, pooled allogeneic MSC population for use as disclosed herein, wherein said shift from the M1 microglia phenotype to the M2 microglia phenotype is calculated as a shift score according to formula 1. Using formula 1, the higher the shift score value the more microglia cell or microglia-like cells with M2 phenotype are present.

The skilled person will appreciate that a shift score may be calculated based on expression of any M1 marker(s) and any M2 marker(s), such as fold increase of CD200R expression and suppression of CD183 expression.

In addition, the upregulation of CX3CL1/Fraktaline and CD200 may be observed on said MSCs when the shift to M2 phenotype of microglia or microglia-like cells occurs. Thus, in one embodiment of the isolated, pooled allogeneic MSC population for use as disclosed herein, said at least 3 assays may further comprise at least one assay measuring the expression of CX3CL1/Fraktaline and CD200 by said MSCs.

In one embodiment, said microglia cells or microglia-like cells are selected from the group consisting of immortalized cell lines, such as the human microglial HMC3 cell line or the CHME-5 cell line; primary microglia obtained from biopsies; primary microglia-like cells cultured from cord blood; and immortalized microglia-like cells from cord blood, such as the DUOC-01 cell line. In one embodiment, said microglia cells or microglia-like cells are selected from the group consisting of immortalized cell lines; such as selected from the group consisting of the HMC3 cell line, CHME-5 cell line and the DUOC-01 cell line.

For example, in some embodiments of the isolated, pooled allogeneic MSC population for use as disclosed herein, said at least three assays comprise one assay measuring indoleamine-2,3-dioxygensase (IDO) activity; one assay measuring prostaglandin E2 secreted by said MSCs; and one assay measuring the effect of the said MSCs on microglia cell and/or microglia-like cells
or one assay measuring indoleamine-2,3-dioxygensase (IDO) activity; one assay measuring the effect of said MSCs on the proliferation of PBMCs; and one assay measuring the effect of the said MSCs on microglia cell and/or microglia-like cells
or one assay measuring prostaglandin E2 secreted by said MSCs; one assay measuring the effect of said MSCs on the proliferation of PBMCs; and one assay measuring the effect of the said MSCs on microglia cell and/or microglia-like cells
or one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs); and one assay measuring the effect of the said MSCs on microglia cell and/or microglia-like cells .

Further assays, such as one assay measuring the effect of said MSCs on the capacity of T cells to suppress an immune response and/or one assay measuring the effect said MCSs on the proliferation and/or apoptosis of dendritic cells may be included. Other additional assays may also be included.

Monocytes originate from myeloid precursors in the bone marrow and they can enter CNS during inflammation. Classically, a monocyte expresses CD14 but not CD16 (referred to as CD14++ CD16- monocytes). These classical monocytes are highly plastic and upon recruitment to inflamed tissues, they can change to macrophages or dendritic cells. Non classical monocytes express CD14 and high levels of CD16 (referred to as CD14+ CD16++ monocytes) and are involved in tissue homeostasis and local regeneration. MSC can change the monocyte phenotype from classical to non-classical. In yet another assay, the monocyte phenotype changes in the presence of said MSCs may be measured. The increasing expression of CD16 and the decreasing percentage of CD14++ CD16- in monocytes in co-culture with and without said MSCs may be compared. The MSCs population which lead to the highest fold induction of CD16 expression and highest suppression of CD14++CD16- is considered most desirable.

Hence, each individual donor derived MSC population may be evaluated in terms of its effect on the monocyte phenotype shift. Thus, in one embodiment, said at least 3 assays further comprise at least one assay measuring the shift from classical to non-classical monocyte phenotype (also referred to as regenerative phenotype) in response to said MSCs, such as in presence of said MSCs. In one embodiment, said at least one assay measures the effect of said MSC on monocyte shift towards regenerative phenotype. In one embodiment, said shift is measured by assaying at least CD16 expression, such as CD16 and CD14 expression in said monocytes.

For example, in some embodiments as disclosed herein, said at least three assays comprise
one assay measuring indoleamine-2,3-dioxygensase (IDO) activity; one assay measuring prostaglandin E2 secreted by said MSCs; and one assay measuring the effect of the said MSCs on monocytes;
or one assay measuring indoleamine-2,3-dioxygensase (IDO) activity; one assay measuring the effect of said MSCs on the proliferation of PBMCs; and one assay measuring the effect of the said MSCs on monocytes;
or one assay measuring prostaglandin E2 secreted by said MSCs; one assay measuring the effect of said MSCs on the proliferation of PBMCs; and one assay measuring the effect of the said MSCs on monocytes;
or one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs); and one assay measuring the effect of the said MSCs on monocytes.

In yet another assay, the HLA-G expression in the MSCs may be measured. HLA-G has been identified as a naturally occurring tolerance-inducing molecule. It has restricted expression under physiological conditions but can be upregulated e.g. in response to IFNγ, IL-10 and PHA. MSC have low levels of intracellular HLA-G and express low levels of soluble HLA-G (sHLA-G) but stimulation with IFNγ or IL-10 is expected to result in increased levels. Stimulation with PHA or GABA is expected to increase soluble HLA-G levels. JEG-3, a placenta derived cell line, has a high level of HLA-G expression, both intracellular and soluble, and may be used as a positive control in the assays. The scope may be to compare both intracellular HLA-G expression for example by flow cytometry (FACS) analysis and the release of sHLA-G by for example ELISA between individual donor derived MSC populations.

Thus, in one embodiment, said at least 3 assays further comprise at least one assay measuring HLA-G expression in said MSCs, for example said at least one assay measures HLA-G expression in said MSCs in response to IFNY, alum, IL-10, PHA and/or GABA, for example said at least one assay measures HLA-G expression in said MSCs in response to IFNY, IL-10 and/or PHA. In one embodiment, said at least one assay measures HLA-G expression in said MSCs in response one or several selected from the group consisting of IFNY, IL-10, PHA and GABA. Said HLA-G expression may be expression of soluble HLA-G.

Additionally, the individual donor derived MSC populations may be evaluated in terms of protein expression and/or cytokine expression in order to select the populations with desired characteristics. For example, it may be of interest to evaluate the expression of interleukins, growth factors, interferon, tumor necrosis factors, colony stimulating factors and lipoproteins in said populations. Thus, in one embodiment, said at least 3 assays further comprise least one assay measuring the protein expression and/or cytokine expression by said MSCs, such as the expression of one or several proteins or cytokines selected from the group consisting of interleukins, growth factors, interferons, tumor necrosis factors, colony stimulating factors and lipoproteins. In another embodiment, said at least one assay measuring the protein expression and/or cytokine expression measures the expression of one or several proteins or cytokines selected from the group consisting of, IL-2, IL-4, IL-6, IL-8, IL-10, IL-12, IL- 12/13, IL-17A, IL-21, IL-22, IL-29, IL-31, TGFβ, VEGF, FGF, GM-CSF (granulocyte-macrophage colony stimulating factor), IFNα, IFNY, apo E and TNFα, such as the group consisting of IL-2, IL-4, IL-6, IL-8, IL-12, IL- 12/13, IL-17A, IL-21, IL-22, IL-29, IL-31, TGFβ, VEGF, FGF, GM-CFS, IFNα, IFNY, apo E and TNFα, such as the group consisting of IL-6, IL-8, GM-CSF and TGFβ, such as the group consisting of at least IL-6.

ACE2 receptor is the entry point on the surface of target cells for infection by coronavirus spike proteins. Co-expression of the serine protease TMPRSS2 by the target cells allows for coronavirus spike protein priming. In one embodiment, said at least one assay measuring the protein expression and/or cytokine expression measures one or several proteins or cytokines selected from the group consisting of IL-2, IL-4, IL-6, IL-8, IL-12, IL- 12/13, IL17A, IL-21, IL-22, IL-29, IL-31, TGFβ, VEGF, FGF, GM-CSF, IFNα, IFNY, apo E and TNFα and ACE2 receptor and TMPRSS2, such as the group consisting of IL-6, IL-8, GM-CSF,TGFβ, ACE2 receptor and TMPRSS2, such as the group consisting of at least ACE2 receptor and TMPRSS2. In one embodiment, least one assay measuring the protein expression measures the expression of ACE2 receptor. In one embodiment, least one assay measuring the protein expression measures the expression of TMPRSS2.

The skilled person will appreciate that also mRNA expression may be measured to evaluate it a given protein is expressed in cells. Thus in one embodiment, said said at least one assay measures the mRNA expression one or several proteins or cytokines selected from the group consisting of IL-2, IL-4, IL-6, IL-8, IL-12, IL- 12/13, IL17A, IL-21, IL-22, IL-29, IL-31, TGFβ, VEGF, FGF, GM-CSF, IFNα, IFNY, apo E and TNFα and ACE2 receptor and TMPRSS2, such as the group consisting of IL-6, IL-8, GM-CSF,TGFβ, ACE2 receptor and TMPRSS2, such as the group consisting of at least ACE2 receptor and TMPRSS2. In one embodiment, least one assay measures the mRNA expression of ACE2 receptor. In one embodiment, least one assay measures the mRNA expression of TMPRSS2.

In one embodiment, each individual donor derived MSC population is negative for the expression of ACE2 receptor and TMPRSS2.

In one embodiment, the isolated, pooled allogeneic MSC population is negative for the expression of ACE2 receptor and TMPRSS2.

In one particular embodiment, the expression of at least 2, such as at least 3, such as at least 4, such as at least 5, such as at least 6, such as at least 7, such as at least 8, such as at least 9, such as at least 10, such as at least 11, such as at least 12, such as at least 13, such as at least 14, such as at least 15, such as at least 16, such as at least 17, such as at least 18, such as all 19 of said proteins and/or cytokines are measured. Furthermore, the skilled person will appreciate the expression of said proteins and/or cytokines may be measured in the absence of any stimuli and/or in the presence of at least one stimulus. In one embodiment, the expression of said proteins and/or cytokines is measured in the presence of at least one stimulus or several stimuli, such as two, three, four or more stimuli. In one embodiment, said stimulus/stimuli is/are immune response modifying stimulus/stimuli. Non-limiting examples of said immune response modifying stimuli include PBMCs; stimulated PBMCs, (such as PBMCs stimulated with PHA, IL10, gamma-aminobutyric acid (GABA), anti-CD2, anti-CD3, anti-CD28, alum and/or interferon gamma (IFNγ)); and/or other. Other non-limiting examples of immune response modifying stimuli include GABA, Poly IC, resiquimod and IFNγ (without addition of PBMCs). Thus, in one embodiment, said immune response modifying stimulus/stimuli is/are selected from the group consisting of PBMCs and stimulated PBMCs, such as PBMCs stimulated with PHA, IL10, gamma-aminobutyric acid (GABA), anti-CD2, anti-CD3, anti-CD28, alum, and/or interferon gamma (IFNγ), such as PBMCs stimulated with PHA, IL10, GABA and/or IFNγ.

In one embodiment, said immune response modifying stimulus/stimuli is/are GABA and/or IFNγ. In one embodiment, there is provided an isolated, pooled allogeneic MSC population for use as disclosed herein, obtainable by a method, wherein the stimulus/stimuli is/are selected from the group consisting of polyinosinic: polycytidylic acid (Poly I:C), resiquimod (r848), GABA and IFNγ, such as the group consisting of Poly I:C and IFNγ or the group consisting of GABA and IFNγ. In one embodiment, said stimuli is PBMCs, such as stimulated or unstimulated PBMCs, such as PHA stimulated PBMCs, such as PHA stimulated T-lymphocytes. In one embodiment, the stimulus /stimuli is/are PHA stimulated T-lymphocytes and/or GABA.

In one particular embodiment, said method may comprise measuring IL-10 expression in said MSCs in response to stimulation with PHA stimulation T-lymphocytes and/or GABA.

In one particular embodiment, said method may comprise measuring expression of tumor necrosis factor-α-induced gene/protein 6 (TSG-6) in said MSCs. TSG-6 has been shown to be involved in reduction of glial scarring.

The skilled person appreciates that said assays may be combined to obtain a specific assay combination of interest depending to the desirable properties of the MSC population(s) assayed. The assays may be selected independently of each other.

It is furthermore of importance that the any MSCs to be pooled to obtain the isolated, allogeneic pooled MSC population for use as disclosed herein and obtainable by said method are cells which have a cell morphology of normal cells. MSC cultures are known to contain a subpopulation of small, round cells that are rapidly self-renewing, usually identified by flow cytometry as low forward scatter and low side scatter. MSCs isolated from donors with greater colony-forming ability are known to have significantly higher proportion of smaller-sized cells. Collectively, data show that donor MSCs classified as having high-growth capacity have an increased capacity for self-renewal, a higher CFU-F efficiency, and a larger proportion of small-sized cells. Cells may be visually inspected during expansion (culture) as well as immediately before or in connection with harvesting and evaluated based on for example the size of cells; size of nuclei; shape of cells; and ratio between cell size and nuclei size. Thus, in one embodiment, said at least 3 assays comprise at least one morphological assay. In one embodiment, said morphological assay assays morphological features of cells and/or cells nuclei. In one embodiment, said morphological features of cells and/or cells nuclei are one or more features selected from the group consisting of the size of the cell, the size of the nuclei, the shape of the cell and the ratio between cell and nuclei size.

It is important that the isolated, pooled allogeneic MSC population for use as disclosed herein comprises as many cells as possible which exhibit healthy and desirable morphology, in other words normal morphology. Thus, in one embodiment, an individual donor derived MSC population is only eligible for pooling if it exhibits at least to 90%, such as at least 91%, such as at least 92%, such as at least 93%, such as at least 94%, such as at least 95%, such as at least 96%, such as at least 97%, such as at least 98%, such at least 99%, normal cells and/or nuclei. Thus, if said individual donor derived MSC population comprises less than 90% normal cells, said population is not eligible for pooling.

It will be appreciated that said step of assaying each individual donor derived MSC population using at least 3 assays may be performed at any of passages 0 (p0) to p8. For example, said assays may be performed when said individual donor derived MSC populations are in the same passage as when they are pooled in order to ensure that said individual donor derived MSC populations then exhibit the desirable properties at the relevant time point. It is also possible that the assays are performed at an earlier passage than the passage at which they are pooled. It will also be appreciated that different assays may be performed at different passages, provided that a particular assay is performed on each individual donor derived MSC population in the same passage to ensure that the assay results obtained for each individual donor derived MSC population may be compared.

Thus, in one embodiment, the step of assaying each individual donor derived MSC population using at least 3 assays is performed when the MSC population is in passage 0 (p0) - passage 8 (p8), such as in p1 - p5, such as in p1 - p4, such as in p2 - p4 or in p1 - p4, such as in p1, p2 and/or p3, such as in p2 and/or p3. In one embodiment, at least one assay, such as at least two assays, such as at least three assays, such as all assays, is/are performed when the cells are in the same passage as when they are pooled. In another embodiment, at least two assays are performed at different passages.

In one embodiment, said each individual donor derived MSC population is assayed by at least one morphology assay.. In one embodiment, said each individual donor derived MSC population is assayed by at least one assay measuring indoleamine-2,3-dioxygensase (IDO) activity. In one embodiment, said each individual donor derived MSC population is assayed by at least one assay measuring the effect of said MSCs on the proliferation of PBMCs. In one embodiment, said each individual donor derived MSC population is assayed by at least one assay measuring prostaglandin E2 secreted by said MSCs.

In one embodiment, said each individual donor derived MSC population is assayed by at least one morphology assay; an assay measuring IDO activity; and an assay measuring the effect of said MSCs on the proliferation of PBMCs. In one embodiment, said each individual donor derived MSC population is assayed by at least a morphology assay; an assay measuring IDO activity; an assay measuring the effect of said MSCs on the proliferation of PBMCs; and an assay measuring prostaglandin E2 secreted by said MSCs. In one embodiment, said each individual donor derived MSC population is further assayed by an assay measuring HLA-G expression in said MSCs. In one embodiment, there is provided an isolated, pooled allogeneic MSC population for use as disclosed herein, obtainable by a method, wherein said each individual donor derived MSC population is further assayed by at least one assay, such as at least two assays, such as at least three assays, such as at least four assays, measuring the expression of at least one, such as two, such as three, such as all four, factor(s) selected from IL-6, IL-8. GM-CSF and TGFβ. It will be understood that each assay may measure the expression of one of IL-6, IL-8. GM-CSF and TGFβ. In one embodiment, said each individual donor derived MSC population is further assayed by an assay measuring HLA-G expression in said MSCs and by at least one assay, such as at least two assays, such as at least three assays, such as at least four assays, measuring the expression of at least one, such as two, such as three, such as all four factor(s) selected from IL-6, IL-8. GM-CSF and TGFβ. It will be understood that each assay may measure the expression of one of IL-6, IL-8, GM-CSF and TGFβ.

The present isolated, pooled allogeneic MSC population for use as disclosed herein, is obtainable by a method which comprises a step of allocating a total score value to each individual donor derived MSC population. In this step a total score value is allocated to each individual donor derived MSC population based on said at least three individual ranking score values. In the case when a higher ranking score value is indicative of more desirable assay result, a higher total score value is indicative of more desirable population properties. Alternatively, in the case when a lower ranking score value is indicative of more desirable assay result, a lower total score value is indicative of more desirable population properties. The skilled person will appreciate that the ranking score value system and/or the total score value system may be modified without departing from the scope of the present disclosure, provided that said systems allow for a comparison between the individual donor derived MSC populations in terms of desirable properties. In one embodiment, the individual ranking score value for at least one assay is allocated to said each individual donor derived MSC population based on a comparison of the assay result for said each individual donor derived MSC population to the results for the remaining individual donor derived MSC populations. Thus, individual ranking score values may be allocated based on comparison between the individual donor derived MSC populations analyzed. In one embodiment, the individual ranking score value for at least one assay is allocated to said each individual donor derived MSC population based on absolute assay result obtained for said individual donor derived MSC population. Thus, a desired threshold value for an assay may be chosen. In one embodiment, the assay result is deemed desirable and an individual ranking score value that reflects the obtained desirable assay result is allocated, when said absolute result corresponds to at least a predetermined value or at most a predetermined value.

It will be appreciated that the step of allocating an individual ranking score value to the results from one, two, three or more of said at least 3 assays involves allocating an individual ranking score value, which individual ranking score value is non binary. A not binary score value is a score value which is selected from at least three levels, in other words at least three different scores. Non limiting examples of non binary score values is 1, 2 and 3; 0, 1 and 2; and 1, 3 and 5. It will be appreciated the non binary ranking score values may be represented by any three numbers X, Y, Z, wherein said X, Y and Z are different numbers. The allocation of non binary score values allows for a higher resolution of ranking the assay results compared to binary score values. Thus, in one embodiment, allocation an individual ranking score value to each individual donor derived MSC population based on the assay result involves allocating a score value selected from at least three ranking score values, such as at least four ranking score values, such as at least five ranking score values. For example, said individual ranking score value may be selected from 5, 6, 7, 8, 9, 10 or even more possible score values. The skilled person will appreciate that the ranking score values may be numeric or not numeric.

The total score value may be an additive score value obtained by addition of ranking score values for each individual donor derived MSC population. Alternatively, the total score value may be a weighed total score value, obtained by 1) assigning a weight to the ranking score value for each assay and 2) adding the weighed ranking score values for individual donor derived MSC population. In this way it is possible to allocate a relatively higher weight (or importance) to one or several assay results of choice compared to the remaining assay results. The skilled person will appreciate that one or several assay results may be weighed and the weight allocated to each assay result may be chosen independently. Thus, in one embodiment, wherein said total score value allocated to said each individual donor derived MSC population is an additive total score value obtained by addition of ranking score values for each individual donor derived MSC population. In another embodiment, said total score value allocated to said each individual donor derived MSC population is a weighed total score value obtained by 1) assigning a weight to the ranking score value for each assay and 2) adding the weighed ranking score values for individual donor derived MSC population.

Based on the total score values, a subset of individual donor derived MSC populations with desirable population properties is selected. In this step, it is envisioned that at least 3, such as at least 4, such as at least 5, such as at least 6, such as at least 7, such as at least 8, such as at least 9, such as at least 10 individual donor derived MSC populations are selected. As used herein, the term “subset” refers to all or fewer than all of the assayed individual donor derived MSC populations.

In one embodiment there is provided an isolated, pooled allogeneic MSC population for use as disclosed herein, obtainable by a method wherein the step of selecting a subset of individual donor derived MSC populations with desirable population properties comprises selecting the individual donor derived MSC populations with total score values which correspond to at least a predetermined total score value in the case wherein a higher total score value is indicative of more desirable population properties; or to at most a predetermined total score value in the case wherein a lower total score value is indicative of more desirable population properties. In another embodiment, the step of selecting a subset of individual donor derived MSC populations with desirable population properties comprises selecting a predetermined number of the individual donor derived MSC populations, which populations exhibit a higher total score value relative the remaining individual donor derived MSC populations in the case wherein a higher total score value is indicative of more desirable population; or which populations exhibit a lower total score value relative the remaining individual donor derived MSC populations in the case wherein a lower total score value is indicative of more desirable population properties.

In the next step of said method, the selected individual donor derived MSC populations are pooled to obtain the isolated, pooled allogeneic MSC population. As explained above, it is considered beneficial that the isolated, pooled allogeneic MSC population comprises similar numbers or numbers in the same range of cells derived from each individual donor, such that cells from one donor are not significantly dominating in said pooled population. Thus, in one embodiment of the isolated, pooled allogeneic MSC population for use as disclosed herein the number of cells derived from any one donor does not exceed about 45%, such as does not exceed about 40%, such as does not exceed about 35%, of the total cell number in said isolated, pooled allogeneic MSC population and said population comprises MCSs derived from at least 3 donors; such as
the number of cells derived from any one donor does not exceed about 40%, such as does not exceed about 35%, such as does not exceed about 30%, of the total cell number in said isolated, pooled allogeneic MSC population and said population comprises MCSs derived from at least 4 donors;
such as the number of cells derived from any one donor does not exceed about 35%, such as does not exceed about 30%, such as does not exceed about 25%, of the total cell number in said isolated, pooled allogeneic MSC population and said population comprises MCSs derived from at least 5 donors;
such as the number of cells derived from any one donor does not exceed about 30%, such as does not exceed about 25%, such as does not exceed about 20%, of the total cell number in said isolated, pooled allogeneic MSC population and said population comprises MCSs derived from at least 6 donors;
such as the number of cells derived from any one donor does not exceed about 25%, such as does not exceed about 22%, such as does not exceed about 20%, of the total cell number in said isolated, pooled allogeneic MSC population and said population comprises MCSs derived from at least 7 donors;
such as the number of cells derived from any one donor does not exceed about 20 %, such as does not exceed about 18 %, such as does not exceed about 16 %, of the total cell number in said isolated, pooled allogeneic MSC population and said population comprises MCSs derived from at least 8 donors;
such as the number of cells derived from any one donor does not exceed about 18 %, such as does not exceed about 15 %, such as does not exceed about 13 %, of the total cell number in said isolated, pooled allogeneic MSC population and said population comprises MCSs derived from at least 9 donors;
such as the number of cells derived from any one donor does not exceed about 16 %, such as does not exceed about 14 %, such as does not exceed about 12 %, of the total cell number in said isolated, pooled allogeneic MSC population and said population comprises MCSs derived from at least 10 donors.

In one particular embodiment, the number of MSC derived from any one individual donor does not exceed about four times, such as about three times, such as about two times the number of the cells derived any other donor.

It will be appreciated that in order to maintain the desired distribution of MSCs derived from individual donors in the population, no further culture of the MCSs is performed after pooling of the selected subset of individual donor derived MSC populations. Without being bound by theory, it is envisioned that the distribution of MSCs derived from individual donors in the pooled population is of importance for obtaining a HLA mismatch expected to ensure no or low HLA immunization in patients administered the isolated, pooled allogeneic MSC population. Therefore, the isolated, pooled allogeneic MSC population as disclosed herein is not further cultured after pooling according to an embodiment. In one embodiment of the present aspect, the isolated poopled allogenein MSC population is not further cultured after the pooling step.

As mentioned above, only individual donor derived MSC populations which fulfill the desired requirements as assayed by said at least said 3 assays are eligible for pooling in the pooling step. The non-eligible cells are thus discarded. It is possible to compare the assay results obtained for the individual donor derived MSC populations, thus the properties of said cells, to the assay results obtained an earlier obtained isolated, pooled allogeneic MSC population, which earlier population was obtained by the method. Hence, the earlier population serves as an internal quality control in the method. Thus, in one embodiment, the method further comprises the step of discarding an individual donor derived MSC population from the pooling step if the assay results for said individual donor derived MSC population are less desirable than the corresponding assay results for a pooled allogeneic MSC population previously obtained by the same method.

Importantly, the method leads to the reduction of variation in the overall assessment of the isolated pooled allogeneic MCS population. To clarify, the variation within a batch is reduced compared at a batch comprising MSC pooled form all donors.

To illustrate, the variation in assessment can be calculated by the following formula:

$$\text{Variation = 1 -} \frac{\text{selected} \left(max - min\right)}{\text{all} \left(max \text{- min}\right)}$$

, wherein the maximum and minimum values are the maximum and minimum assay results obtained.

The overall assessment may be calculated as the delta selection algorithm score for selected donors, in this example 6-3 =3, divided by the delta selection algorithm score for all donors evaluated, in this example 6-1 = 5.

$$\text{Variation =} \frac{\text{1 -selected} \left(max - min\right)}{\text{all} \left(max \text{- min}\right)} = \frac{1 - 3}{5} = 0.4$$

Thus, in one embodiment of the isolated, pooled allogeneic MSC population for use as disclosed herein, the the variation in the overall assessment within a batch is reduced by at least 30%, such as at least 35 %, such as at least 40 % such as at least 45 %, such as at least 50 %, such as at least 60 %, when comparing the assay results for all the individual donor derived MSC populations assayed and the for selected a subset of individual donor derived MSC populations.

As explained in detail in the present disclosure, the method by which the isolated, pooled allogeneic MSC population for use as disclosed herein is obtainable allows for obtaining batches of isolated, pooled allogeneic MSC population as disclosed herein, which batches show no statistically significant batch-to-batch variability.

In another embodiment, said method further comprises the step of culturing the isolated, pooled allogeneic MSC population in the presence of proinflammatory compound(s), such as IFNγ, alum and/or tumor necrosis factor alpha for a period prior to administration to a patient in need thereof, for example for at least 12 hours but not for more than 72 hours, such as 24-72 hours. For example, said culture step may be performed for a period of from about 12 to about 72 hours. For example, said period may be about 24 hours, 36 hours, 48 hours, 60 hours or about 72 hours. Said period may be any period from about 12 hours to about 72 hours. In one embodiment, the culture step is performed directly prior to administration. In some embodiments as disclosed herein, said culture period ends no more than about 12, such as 11, 10, 9, 8, 7, 6, 5, 4, 3, 2 or 1 hour(s) prior to administration.

As explained in above, pooling of the MSCs is restricted to the formulation step of obtaining the isolated, pooled allogeneic MSC population and hence the cells are not subject to any culture or additional expansion after pooling. This ensures no additional expansion of the cells and thus no associated negative impact of such expansion on the potency and functionality of the isolated, pooled allogeneic MSC population. Culture after pooling increases the risk for negative impact, such as, but not limited to, a loss of immunosuppressive and/or immune-modulatory potential and an increase in inflammatory markers. Additionally, if cells are cultured/expanded after pooling the negative impact of loss of immunosuppressive and/or immune-modulatory potential and/or increase in inflammatory markers may be differential across pooled batches, therefore indicating a negative impact on batch-to-batch variability. Data from the inventors demonstrate that pooling of the MSCs according to the present disclosure, without further expansion of the cells can lead to an enhanced immunosuppressive and/or immune-modulatory potential compared to the single donor cells of which the pooled product is comprised. Thus, the isolated, pooled allogeneic MSC population for use as disclosed herein exhibits desirable properties. Thus in one embodiment said isolated pooled allogeneic MSC population for use as disclosed herein, wherein said pooled population exhibits enhanced immunosuppressive and/or immune-modulatory potential compared to individual donor derived MSC populations. Said comparison may be with said at least 3 individual donor derived MSC populations assayed as defined in the inventive method, such as each individual donor derived MSC population assayed. Thus, said pooled population may exhibit enhanced immunosuppressive and/or immune-modulatory potential compared to at least approximately 50 %, such as approximately 60 %, such as approximately 70 %, such as approximately 75 %, such as approximately 80 %, such as approximately 85 %, such as approximately 90 %, such as approximately 95%, such as approximately 100 % of the assayed individual donor derived MSC populations. Alternatively, said comparison may be with said the individual donor derived MSC populations selected for pooling as defined in the inventive method. Thus, said pooled population may exhibit enhanced immunosuppressive and/or immune-modulatory potential compared to at least approximately 50 %, such as approximately 60 %, such as approximately 70 %, such as approximately 75 %, such as approximately 80 %, such as approximately 85 %, such as approximately 90 %, such as approximately 95%, such as approximately 100 % of the individual donor derived MSC populations selected for pooling.

In one embodiment, said pooled population for use as disclosed herein exhibits enhanced immunosuppressive and/or immune-modulatory potential compared the assayed individual donor derived MSC populations, wherein said enhancement is by at least approximately 5 %, such as at least approximately 7.5 %, such as at least approximately 10 %, such as at least approximately 12.5 %, such as at least approximately 15 %, such as at least approximately 17.5 %, such as at least approximately 20 %, such as at least approximately 22.5 %, such as at least approximately 25 %, such as at least approximately 30 % or more.

It will be appreciated by the skilled person that the above listed embodiments may be combined in any way. The different combinations are not listed here individually merely for the sake of brevity, but are represented here in a table with columns A, B and C. It will be appreciated that any value (row) in column A and/or B may be combined with any value (row) in column C, to arrive at an embodiment as disclosed herein. The selection of values from columns A, B and C is an independent selection. Thus said embodiments can be expressed as said pooled population may exhibit enhanced immunosuppressive and/or immune-modulatory potential compared to [value from column A] of the assayed individual donor derived MSC populations and/or [value from column B] of the individual donor derived MSC populations selected for pooling, wherein said enhancement is [value for column C]. Non-limiting illustrative examples include: an embodiment, wherein said pooled population exhibits enhanced immunosuppressive and/or immune-modulatory potential compared to at least approximately 50 % of the assayed individual donor derived MSC populations, wherein said enhancement is by at least approximately 10 %; as well as an embodiment, wherein said pooled population exhibits enhanced immunosuppressive and/or immune modulatory potential compared to at least approximately 75 % of individual donor derived MSC populations selected for pooling, wherein said enhancement is by at least approximately 5 %.

Table illustrating combinations of the above mentioned embodiments.

| A | B | C |
|---|---|---|
| % of the assayed individual donor derived MSC populations | % of the individual donor derived MSC populations selected for pooling | Level of enhancement compared to A or B |
| At least approximately 50% | At least approximately 50% | At least approximately 5% |
| At least approximately 60% | At least approximately 60% | At least approximately 10 % |
| At least approximately 70% | At least approximately 70% | At least approximately 12.5% |
| At least approximately 75% | At least approximately 75% | At least approximately 15% |
| At least approximately 80% | At least approximately 80% | At least approximately 17.5% |
| At least approximately 85% | At least approximately 85% | At least approximately 20% |
| At least approximately 90% | At least approximately 90% | At least approximately 22.5% |
| At least approximately 95% | At least approximately 95% | At least approximately 25% |
| Approximately 100 % | Approximately 100 % | At least approximately 30% |

In one particular embodiment, said enhanced immunosuppressive and/or immune-modulatory potential is measured as expression of IDO by unstimulated MSC. In another embodiment, said enhanced immunosuppressive and/or immune-modulatory potential is measured as expression of PGE2 by unstimulated MSC.

In one embodiment, said isolated, pooled allogeneic MSC population for use as disclosed herein comprises MSCs from at least 3, such as at least 4, such as at least 5, such as at least 6, such as at least 7, such as at least 8, such as at least 9, such as at least 10 individual donors. For example, said population may contain MSCs from 3-10, such as 4-10, such as 5-10, such as 5-9, such as 5-8, such as from 5, 6 or 7 individual donors. Furthermore, in one embodiment, said isolated, pooled allogeneic MSC population for use as disclosed herein exhibits no statistically significant batch-to-batch variability. The method allows for obtaining an isolated, pooled allogeneic MSC population which exhibits advantageous properties. It is considered advantageous that large batches of MSCs may be obtained due to the step of pooling cells, and additionally this allows for reduction of manufacturing costs. Due to the pooling of individual donor derived MSC populations, it is possible to maintain the cells at low passage numbers as described above, whereby the obtained isolated, pooled allogeneic MSC population exhibits high potency as well as low risk of genetic instability. Hence, large batches of genetically stable cells with high potency can be obtained. Furthermore, the isolated, pooled allogeneic MSC population obtainable by the method as disclosed herein exhibits no statistically significant batch-to-batch variability, due to the method steps employed.

As used herein, the term “batch” refers to an isolated, pooled allogeneic MSC population obtained by the method as disclosed herein.

As used herein, the term “batch-to-batch variability” refers to the difference in properties between an isolated, pooled allogeneic MSC population obtained by the method as disclosed herein and another isolated, pooled allogeneic MSC population obtained by the method as disclosed herein.

Said batch-to-batch variability may be quantified by comparing the results from one or several of said at least three assays which were used for assaying the individual donor derived MSC populations. Alternatively, one or several different assays may be employed.

As used herein, the term “no statistically significant batch-to-batch variability” is to be interpreted as the difference between the assay results from one batch and the assay results from a different batch is not statistically significant (for example using a probability value of P>0.05). Said statistical significance may be justified as the coefficient of variance between batches is equal or below the inter and/or intra assay coefficient of variance. The skilled person is familiar suitable statistical calculations.

Thus, in one embodiment, there is provided an isolated, pooled allogeneic MSC population for use as disclosed herein, which population exhibits no statistically significant batch-to-batch variability. In one embodiment, said no statistically significant batch-to-batch variability is between two consecutively produced batches. In one embodiment, said no statistically significant batch-to-batch variability is between any two batches, for example such as two consecutively produced batches or for example such as any two batches produced 1, 2, 3, 4, 5, 6, 7, 8, 9, 10 or even more batches apart. In one embodiment, said no statistically significant batch-to-batch variability is between a produced batch and reference batch, wherein said reference batch is an isolated, pooled allogeneic MSC population previously produced by the method as disclosed herein.

In one embodiment, said no statistically significant batch-to-batch variability is associated with the probability value (P) of > 0.05, such as for example P of > 0.04, such as for example P of > 0.03, such as for example P of > 0.02, such as for example P of > 0.01, such as for example P of > 0.005, such as for example P of > 0.001.

In one embodiment, said batch-to-batch variability is quantified based on the assay results from at least 2 of said 3 assay selected from the group consisting of the IDO assay as described herein, the PGE2 assay as described herein and the proliferation assay as described herein; and at least 1 assay selected from the group consisting of the Treg assay as described herein, the DC assay as described herein, the monocyte assay as described herein and the microglia assay as described herein; such as all three of the IDO assay as described herein, the PGE2 assay as described herein and the proliferation assay as described herein and the microglia assay as described herein. Optionally, the batch-to-batch variability may be quantified based one or more additional assays.

As explained herein,, it may be beneficial that the isolated, pooled allogeneic MSC population for use as disclosed herein is derived from a native MSC source in contrast to a transdifferentiated or dedifferentiated MSC source, for example for reasons of epigenetic memory. Furthermore, an isolated, pooled allogeneic MSC population as disclosed herein derived from a native MSC source may be beneficial for reasons of safety, such as lower risk of tumorigenicity or ectopic tissue formation. It is known that that, unless terminally differentiated, cells can transform and become malignant in vivo, for example via the formation of tumours or ectopic tissue. In contrast, this has not been observed for MSCs derived from native human MSC sources. Thus, in one embodiment of the present aspect, said MSCs isolated, pooled allogeneic MSC population as disclosed herein is obtained from a native MSC source. Such native sources are disclosed in connection with the first aspect as disclosed herein and will not be repeated here for the mere sake of brevity.

The isolated, pooled allogeneic MSC population for use as disclosed herein exhibits desired functional and morphological properties, high potency, no statistically significant batch-to-batch variability and is also obtainable in large batches. This allows for predictability and low variability when said population is used as a medicament. Thus, the present isolated, pooled allogeneic MSC population for use as disclosed herein may be used in a standardized medical treatment procedure. It is envisioned that there are both logistic and dosing advantages for the isolated, pooled allogeneic MSC population obtainable by the method as disclosed herein, when said population is used as medicament, in particular in regard to the formulation and dose regimen. The logistic chain is to keep said isolated, pooled allogeneic MSC population in cryogenic storage, hence ensuring that the properties of the isolated, pooled allogeneic MSC population are maintained and allowing that the patient receives a predefined cell number as a medicament, in contrast to “giving the patient the number of cells we managed to expand” according to the prior art, is considered important. Thus, the isolated, pooled allogeneic MSC population as disclosed herein is suitable as on “off the shelf” standardized medical product, which offers predictability in terms of therapeutic effect and safety.

It is envisioned that said isolated, pooled allogeneic MSC population as disclosed herein will be useful in the treatment and/or prevention of diseases or conditions selected from the group consisting of inflammatory diseases or conditions, autoimmune disease, transplantation rejection and CNS disorders. In particular, said isolated, pooled allogeneic MSC population as disclosed herein may be exposed to one or several stimulating factors, for example pro inflammatory factors and/or factors stimulating the immunosuppressive capacity said population, prior to administration to a subject in need thereof. For example, said stimulating factors may be IFNγ and/or tumor necrosis factor alpha and/or alum.

In one embodiment, said isolated, pooled allogeneic MSC population for use as disclosed herein, is exposed to IFNγ or/and tumor necrosis factor alpha and/or alum hours prior to administration, such as directly prior to administration. For example, said exposure may be for a period of from about 1 to about 24 hours prior to administration, such as directly prior to administration. For example, said exposure period may be about up to 1 hour or about 1, 2, 4, 5 or 24 hours. Said period may be any period any period of about 24 hours or less. For example, in some embodiments said period may be less than about 1 hour (in other words up to about 1 hour). Said period may be any period from up to about 1 hour (in other words less than about 1 hour) to about 24 hours or from about 1 hour to about 24 hours . In some embodiments, said exposure ends no more than about 12, such as 11, 10, 9, 8, 7, 6, 5, 4, 3, 2 or 1 hour(s) prior to administration. It will be appreciated by the person skilled in the art that said exposure occurs prior to administration of the cells to a patient in need thereof and is not to be equated with further culturing of the isolated, pooled allogeneic MSC population after pooling. The purpose of said exposure is to induce expression of factors beneficial for treatment of the disorder in the patient and is not for the purpose of proliferation of cells in order to obtain a larger cell population. It will be appreciated that said exposure does not affect batch to batch variability. In the case wherein said isolated, pooled allogeneic MSC population after pooling is frozen after pooling (such a frozen directly after pooling without any additional culture after the pooling step), the exposure as discussed herein is after thawing the isolated, pooled allogeneic MSC population but prior to administration to the patient. The skilled person will appreciate that the step of exposing said isolated, pooled allogeneic MSC population is different from cell culture for the expansion of the cell population. Therefore, the exposure in this context is for a shorter period of time than the average doubling time of the cells. Without being bound by theory, the isolated, pooled allogeneic MSC population is envisioned to be able modulate responses by innate and adaptive immune cells, retain dendritic cells in an immature state, inhibit dendritic cell differentiation and suppressing their proinflammatory cytokine production. Therefore, the present inventive isolated, pooled allogeneic MSC population is envisioned to be useful for the treatment and/or prevention of inflammatory and autoimmune diseases or conditions, transplant rejections as well as CNS disorders., such as amylotrophic lateral sclerosis (ALS), primary lateral sclerosis (PLS), progressive muscular atrophy (PMA), multiple sclerosis (MS), cerebral palsy (CP), hypoxia related brain damage, diffuse cerebral sclerosis of Schilder, acute disseminated encephalomyelitis, acute hemorrhagic leukoencephalitis, transverse myelitis and/or neuromyelitis optica, in particular such as amylotrophic lateral sclerosis (ALS), primary lateral sclerosis (PLS), progressive muscular atrophy (PMA), multiple sclerosis (MS), cerebral palsy (CP) and/or hypoxia related brain damage; such as amylotrophic lateral sclerosis (ALS), primary lateral sclerosis (PLS), and/or progressive muscular atrophy (PMA).

COVID-19 infection can induce a range of neurological symptoms, indicating the potential for the SARS-CoV-2, as well as, other members of the coronavirus family to target the central nervous system. More extensive research on coronavirus infections have demonstrated neurological manifestations such as febrile seizures, convulsions and encephalitis. Current research indicates that the virus can enter the CNS through the olfactory bulb, resulting in inflammation and demyelination. Without being bound by theory, the present inventors envision that the ability of MSC therapy to target inflammatory processes through modulation of the immune cell compartment and induction of immune tolerance indicates potential for stromal cell therapy to be of value in COVID-19 treatment and in the treatment of long-term neurological complications associated with COVID or coronavirus infection (Heneka et al., 2020). Likewise, the current use of MSC therapy in neurological disorders such as multiple sclerosis illustrate the potential for stromal therapeutics in targeting of demyelinating conditions such as COVID-19.

Therefore, in one embodiment of the isolated pooled allogeneic MSC population for use as described herein, the the treatment and/or prevention of COVID-19 infection, is the treatment and/or prevention of neurological symptoms associated with COVID-19 infection, such as inflammation or demyelination associated with COVID-19 infection.

Treatment options for COVID-19 are constantly evolving, with options including repurposed drugs such as dexamethasone, convalescent plasma, immunoglobulin based treatment. Despite growing treatment options, the burden of long COVID, a continuing manifestation of the disease symptoms after the virus has left the system, is extensive. Many of these long COVID patients present with neurological symptoms including loss of memory, concentration and sleep related issues. Neurological effects of COVID infection have not been as extensively investigated as the respiratory associated symptoms. However, there have been reports of infected patients experiencing delirium, swelling and inflammation of the brain and a deterioration in myelin, as seen in multiple sclerosis. Neurological effects have since been widened to include stroke and brain hemorrhage, peripheral nerve damage, anxiety and post-traumatic stress disorder (Marshall. 2020 Nature. 585; 342-343) In one embodiment, said use is in the treatment and/or prevention of neurological symptoms associated with COVID-19 infection is the use in the treatment and/or prevention of inflammation associated with COVID-19 infection. In one embodiment, said use is in the treatment and/or prevention of neurological symptoms associated with COVID-19 infection is the use in the treatment and/or prevention of demyelination associated with COVID-19 infection. In one embodiment, there is provided an isolated, pooled allogeneic MSC population for use as disclosed herein, for use in the treatment and/or prevention of symptoms associated with long COVID-19 infection, such as for use in the treatment and/or prevention of neurological symptoms associated with long COVID-19 infection.

As used herein, the term “long COVID-19” refers to persistence of symptoms associated with COVID-19 infection post-resolution of viral infection. As used herein, the term “neurological symptoms associated with long COVID-19 infection” refers to persistence of neurological symptoms associated with COVID-19 infection post-resolution of viral infection.

As used herein, the term “infusion” is meant to be interpreted as encompassing infusion and injection. Thus, for example, term “intrathecal infusion” encompasses “intrathecal injection”.

In one embodiment, said use comprises administration of said isolated, pooled allogeneic MSC population as an infusion to patient in need thereof. In one embodiment, said infusion/injection is administered intravenously, intraperitoneally or intralymphatically, intravenously, intrathecal, intracerebral and or through the ommaya reservoir, intraarterially or subcutaneously. In one embodiment, said infusion/injection is administered intravenously, intraperitoneally, intralymphatically, intravenously, intrathecal, intracerebral, through the ommaya reservoir, intraarterially or subcutaneously. In one embodiment, said infusion/injection is administered intravenously, intraperitoneally or intralymphatically, intravenously, intrathecal, intracerebral, through the ommaya reservoir, intraarterially, subcutaneously or intramuscularly. In one embodiment, said infusion or injection is administered intramuscularly. In one embodiment, said infusion is administered intravenously, intraperitoneally or intralymphatically, such as intravenously. In one embodiment, said administration is intravenous infusion or intravenous injection.

Proposed regenerative approaches to neurological diseases using MSCs include cell therapies in which cells are delivered via intracerebral or intrathecal infusion/injection. Thus, in one embodiment,, said use comprises administration of said isolated, pooled allogeneic MSC population as an intrathecal or intracerebral infusion/injection, such as an intrathecal infusion/injection. Relevant mechanisms of action after transplantation of MSCs into the brain include that MSCs promote neurogenesis, decrease apoptosis and necrosis, reduce levels of free radicals, encourage synaptic connection from damaged neurons, release diverse neurotrophic factors and regulate inflammation, reduce vascular injury, primarily through paracrine actions, (Joyce, 2010, Regen Med. Nov;5(6):933-46;Lima et al., 2020, Stem Cell Res Ther. 11:367).

In another embodiment, said use comprises administration of said isolated, pooled allogeneic MSC population as an intravenous infusion/injection. In another embodiment, said use comprises administration of said isolated, pooled allogeneic MSC population as an intramuscular infusion/injection.

The modulatory nature of MSC and the low expression of HLA-DR are two reasons for assuming that allogeneic transplantation of MSC may be accepted without HLA matching between donor and recipient. It is envisioned that, said infusion/injection may be performed repeatedly or only once, depending on the therapeutic needs of the patient. Without being bound by theory, it is envisioned that said administration, by one infusion/injection or repeated infusions/injections, will not lead to clinically relevant levels of anti-HLA antibodies in the treated patients. Hence, said patients will be eligible for several infusion/injection treatments as described herein. Thus, in one embodiment, said I infusion/injection is performed only once. In another embodiment, said infusion/injection is performed repeatedly. For example, said infusion/injection may be performed two times, three times, four times or more. Said infusion/injection may for example be performed every month, every two months, every three months, every fourth month, every fifth month or every six month or more.

In particular, in the embodiments wherein said isolated, pooled allogeneic MSC population as disclosed herein is for use in the treatment and/or prevention of a CNS disorder as disclosed herein, infusion/injection may be performed every month, every two months, every three months, every fourth month, every fifth month or every six month or more. It is envisioned that said treatment may be continued throughout the life span of the patient in need thereof.

Thus, in one embodiment, said administration induces no or low anti-HLA antibody titers in said patient. As used herein, the term “low or no anti-HLA antibody titers” refers to titers which are considered clinically irrelevant. Antibody analysis by solid phase multiplex technologies have allowed for a more precise definition of the breadth and strength of HLA antibodies. By correlating these results with those obtained by an actual cell-based crossmatch, and eventual graft outcome, clinically relevant antibodies can be defined in a center-specific manner (Zachary et al. Hum Immunol 2009; 70: 574-579). The skilled person will appreciate that clinically irrelevant anti-HLA antibody titers may be defined by LABScreen single antigen beads test with higher mean fluorescence intensity (MFI) for donor specific antibodies than 1000, DSA MFI >1000.

The isolated, pooled allogeneic MSC population for use as described herein is to be administered in a therapeutically effective dose. In one embodiment, there is provided an isolated, pooled allogeneic MSC population for use as discloses herein, wherein said use comprises administration to said patient a dose of approximately at least 3 × 10⁶ cells, such as approximately at least 5 × 10⁶ cells, such as approximately at least 10 × 10⁶ cells, such as approximately at least 15 × 10⁶ cells, such as approximately at least 20 × 10⁶ cells, such as approximately at least 25 × 10⁶ cells, such as approximately at least 30 × 10⁶ cells, such as approximately at least 50 × 10⁶ cells,, such as approximately at least about 60 × 10⁶ cells, such as approximately at least about 75 × 10⁶ cells, such as approximately at least about 100 × 10⁶ cells, such as approximately at least about 150 × 10⁶ cells, such as at least approximately at least about 200 × 10⁶ cells . In one embodiment, said use comprises administration to said patient a dose of approximately at least 0.1 × 10⁶ cells/kg bodyweight, such as approximately at least 0,3 × 10⁶ cells/kg bodyweight, such as approximately at least 0,5 × 10⁶ cells/kg bodyweight, such as approximately at least 0,75 × 10⁶ cells/kg bodyweight, such as approximately at least 1 × 10⁶ cells/kg bodyweight, such as approximately at least 1,2 × 10⁶ cells/kg bodyweight. In one embodiment, said use said use comprises administering to said patient a dose from approximately 0.1 × 10⁶ cells/kg bodyweight to approximately 10 × 10⁶ cells/kg bodyweight, such as from approximately 0.15 × 10⁶ cells/kg bodyweight to approximately 4 × 10⁶ cells/kg bodyweight, such as from approximately 0.20 × 10⁶ cells/kg bodyweight to approximately 4 × 10⁶ cells/kg bodyweight, such as from approximately 0.25 × 10⁶ cells/kg bodyweight to approximately 4 × 10⁶ cells/kg bodyweight, such as from approximately 0.3 × 10⁶ cells/kg bodyweight to approximately 4 × 10⁶ cells/kg bodyweight, such as for example from approximately 0.25 × 10⁶ cells/kg bodyweight to approximately 3 × 10⁶ cells/kg bodyweight, such as from approximately 0.25 × 10⁶ cells/kg bodyweight to approximately 2 × 10⁶ cells/kg bodyweight or from approximately 0.3 × 10⁶ cells/kg bodyweight to approximately 1.2 × 10⁶ cells/kg bodyweight.

It is envisioned that said isolated, pooled allogeneic MSC population for use as disclosed herein will be useful as a pharmaceutical composition.

Thus, in a second aspect of the present disclosure, there is provided a pharmaceutical composition comprising an isolated, pooled allogeneic MSC population as disclosed herein and at least one pharmaceutically acceptable excipient or carrier. Said pharmaceutical composition may be useful a medicament, for example for treatment and or prevention of COVID-19 infection, such as of neurological symptoms associated with COVID-19 infection, such as inflammation or demyelination associated with COVID-19 infection.

It will be appreciated that the pharmaceutical composition may be useful in the treatment and/or prevention of any one of the diseases or conditions listed in connection with the fourth aspect of the present disclosure, which diseases or conditions will for the sake of brevity not be repeated here. In one embodiment, said pharmaceutical composition comprises approximately at least 3 × 10⁶ cells, such as approximately at least 5 × 10⁶ cells, such as approximately at least 10 × 10⁶ cells, such as approximately at least 15 × 10⁶ cells, such as approximately at least 20 × 10⁶ cells, such as approximately at least 25 × 10⁶ cells, such as approximately at least 30 × 10⁶ cells, such as approximately at least 50 × 10⁶ cells, such as approximately at least about 60 × 10⁶ cells, such as approximately at least about 75 × 10⁶ cells, such as approximately at least about 100 × 10⁶ cells, such as approximately at least about 150 × 10⁶ cells, such as at least approximately at least about 200 × 10⁶ cells. Thus, one dosage of said composition comprises the above mentioned number of cells.

In one embodiment, said pharmaceutical composition comprises an isolated, pooled allogeneic MSC population as disclosed herein, wherein said population has not be subject to further culture after the pooling.

In another embodiment, said pharmaceutical composition comprises an isolated, pooled allogeneic MSC population as disclosed herein, wherein said population has been exposed to IFN-γ or/and tumor necrosis factor alpha and/or alum for a period prior to administration, such as directly prior to administration. Said population may be exposed for a period of about 24 hour or less. For example, in some embodiments said period may be less than about 1 hour (in other words up to about 1 hour). For example, said population may be exposed for a period of from up to about 1 hour (in other words less than about 1 hour) to about 24 hours or from about 1 to about 24 hours prior to administration, such as directly prior to administration. For example, said exposure period may be up to about 1 hour, about 1 hour, 4 hours, 6 hours, 12 hours or about 24 hours. Said period may be any period from about 12 hours to about 24 hours. In some embodiments, said culture ends no more than about 12, such as about 11, 10, 9, 8, 7, 6, 5, 4, 3, 2 or 1 hour(s) prior to administration.

In one embodiment, said pharmaceutical composition is formulated for infusion/injection; such for intravenous infusion/injection, intraperitoneal infusion/injection, intralymphatical infusion/injection, intravenous infusion/injection, intracerebral infusion/injection, intrathecal infusion/injection, intracerebral infusion/injection, intraarterial infusion/injection, subcutaneous infusion/injection or infusion/injection through the ommaya reservoir; such as for intracerebral or intrathecal infusion/injection. In one embodiment, said pharmaceutical composition is formulated for intravenous infusion/injection. In one embodiment, said pharmaceutical composition is formulated for intravenous infusion or intravenous injection.

In a third aspect of the present disclosure, there is provided a method for treatment and/or prevention of a disease or condition, which disease or condition is or is associated with COVID-19 infection, comprising the steps of
-obtaining an isolated, pooled allogeneic mesenchymal stem/stromal cell (MSC) population using the method as defined herein; and
-administering a therapeutically effective dose of an isolated, pooled allogeneic MSC population as disclosed herein or of a pharmaceutical composition comprising said isolated, pooled allogeneic MSC population to a patient in need thereof.

In one embodiment, said disease or condition is or is associated with COVID-19 infection is inflammation associated with COVID-19 infection. In one embodiment, said disease or condition is or is associated with COVID-19 infection is demyelination associated with COVID-19 infection.

The skilled person will appreciate that any embodiments mentioned in connection with the first aspect of the present disclosure are equally applicable to the inventive method of treatment and/or prevention. For the sake of brevity, said embodiments will not be repeated here or will only be mentioned briefly. In one embodiment of said method, the administration of said MSC population is by infusion/injection, such as intravenous infusion/injection, intraperitoneal infusion/injection, intralymphatical infusion/injection, intravenous infusion/injection, intrathecal infusion/injection, intracerebral infusion/injection, intraarterial infusion/injection, subcutaneous infusion/injection or infusion/injection through the ommaya reservoir. In one embodiment of said method, the administration is by intravenous, intraperitoneal or intralymphatic infusion/injection. In one embodiment of said method, the administration is by intrathecal infusion/injection or intracerebral infusion/injection. In one embodiment of said method, the administration is by intravenous infusion/injection. In one embodiment of said method, the administration is by intravenous infusion or intravenous injection.

In one embodiment, said infusion is performed repeatedly. In another embodiment, said infusion/injection is performed one time only. In one embodiment of the method for treatment and/or prevention as disclosed herein, said administration induces no or low anti-HLA antibody titers in the patient. In one embodiment, said method comprises administering to said patient a dose of approximately at least 3 × 10⁶ cells, such as approximately at least 5 × 10⁶ cells, such as approximately at least 10 × 10⁶ cells, such as approximately at least 15 × 10⁶ cells, such as approximately at least 20 × 10⁶ cells, such as approximately at least 25 × 10⁶ cells, such as approximately at least 30 × 10⁶ cells, such as approximately at least 50 × 10⁶ cells,, such as approximately at least about 60 × 10⁶ cells, such as approximately at least about 75 × 10⁶ cells, such as approximately at least about 100 × 10⁶ cells, such as approximately at least about 150 × 10⁶ cells, such as at least approximately at least about 200 × 10⁶ cells. In one embodiment, said method comprises administering to said patient a dose of approximately at least 0.1 × 10⁶ cells/kg bodyweight, such as approximately at least 0,3 × 10⁶ cells/kg bodyweight, such as approximately at least 0,5 × 10⁶ cells/kg bodyweight, such as approximately at least 0,75 × 10⁶ cells/kg bodyweight, such as approximately at least 1 × 10⁶ cells/kg bodyweight, such as approximately at least 1,2 × 10⁶ cells/kg bodyweight. In one embodiment, said method comprises administering to said patient a dose approximately from about 0.1 × 10⁶ cells/kg bodyweight to about 10 × 10⁶ cells/kg bodyweight, such as from about 0.15 × 10⁶ cells/kg bodyweight to about 4 × 10⁶ cells/kg bodyweight, such as from about 0.20 × 10⁶ cells/kg bodyweight to about 4 × 10⁶ cells/kg bodyweight, such as from about 0.25 × 10⁶ cells/kg bodyweight to about 4 × 10⁶ cells/kg bodyweight, such as from about 0.3 × 10⁶ cells/kg bodyweight to about 4 × 10⁶ cells/kg bodyweight, such as for example from about 0.25 × 10⁶ cells/kg bodyweight to about 3 × 10⁶ cells/kg bodyweight, such as from about 0.25 × 10⁶ cells/kg bodyweight to about 2 × 10⁶ cells/kg bodyweight or from about 0.3 × 10⁶ cells/kg bodyweight to about 1.2 × 10⁶ cells/kg bodyweight.

In yet another aspect of the present disclosure, there is provided a use of the isolated, pooled allogeneic MSC population as disclosed herein, in the manufacture of a medicament for the treatment and/or prevention of COVID-19 infection or of symptoms associated with COVID-19 infection.

In one embodiment of this aspect, said use is in the manufacture of a medicament for the treatment and/or prevention of neurological symptoms associated with COVID-19 infection. In one embodiment of this aspect, said use is in the manufacture of a medicament for the treatment and/or prevention of inflammation associated with COVID-19 infection. In one embodiment of this aspect, said use is in the manufacture of a medicament for the treatment and/or prevention of demyelination associated with COVID-19 infection. In one embodiment of this aspect, said use is in the manufacture of a medicament for the treatment and/or prevention of symptoms associated with long COVID-19 infection, such as neurological symptoms associated with long COVID-19 infection. It will be understood that any embodiments listed in connection to the first aspect as disclosed herein are equally relevant for this aspect and are not repeated here merely for the sake of brevity.

In another aspect of the present disclosure, there is provided a method for evaluating of potency of a MSC population, comprising the step of:
culturing or providing a MSCs population;
assaying said MSC population using at least 3 assays to obtain said at least 3 assay results;
for each assay allocating a score value to said MSC population based on the assay result, wherein a higher score value is indicative of more desirable assay result; or wherein a lower score value is indicative of more desirable assay result;
allocating a total score value to said MSC population based on the score values allocated to each assay, wherein in the case of a higher score value being indicative of more desirable assay result, a higher total score value is indicative of more desirable population properties; or wherein in the case of a lower score value being indicative of more desirable assay result, a lower total score value is indicative of more desirable population properties;
qualifying the MSC population as potent if said total score value is above a predetermined threshold value in the case of a higher score value being indicative of more desirable assay result, or qualifying the MSC population as potent if said total score value is below a predetermined threshold value in the case of a lower score value being indicative of more desirable assay result. In one embodiment, said at least 3 assays comprise wherein 2 of said at least 3 assays are selected from the group consisting of one assay measures indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs) and
wherein 1 of said at least 3 assays is selected from the group consisting of one assay measuring the effect of said MCSs on the capacity of T cells to suppress an immune response; one assay measuring the effect said MCSs on the proliferation and/or apoptosis of dendritic cells; one assay measuring the effect of the said MSCs on monocytes and one assay measuring the effect of the said MSCs on microglia cell and/or microglia-like cells. In one embodiment, the method employs the assays as disclosed in the context of the first aspect described herein.

The isolated, pooled allogeneic MSC population may furthermore be useful for culturing cells to be used in ex vivo therapy, for example the MSC population may be used as feeder cells or to providing factor or signals of interest. Thus, in yet another aspect of the present disclosure the use of the isolated, pooled allogeneic MSC population as disclosed herein for co-culture of immune cells is provided. For example, said MSC population may be used as feeder cells in culture for ex vivo expansion and/or stimulation of immune cells, for example but not limited to dendritic cells, natural killer cells, lymphocytes (such as B-cells or T-cells), monocytes and mast cells. Said MSC population may be used as exosome producing cells and/or paracrine factor producing cells and/or for cell to cell stimulation between MSC and immune cells in culture. In one embodiment, there is provided the use of the isolated, pooled allogeneic MSC population as disclosed herein as feeder cells for co-culture of immune cells. In one embodiment, there is provided the use of the isolated, pooled allogeneic MSC population as disclosed herein for stimulation of immune cells co-cultured with said population. It is envisioned that said immune cells which have been co-cultured with the isolated, pooled allogeneic MSC population as disclosed herein will be useful in therapy.

### Definitions of Tests

As used herein, the following tests are performed in the disclosed clinical trial and the skilled person is familiar with said tests:
Modified Ashworth Spasticity scale (MAS) measures resistance during passive soft-tissue stretching and is used as a simple measure of spasticity. Muscle tone of bilateral elbows and ankles will be quantified using the Modified Ashworth Spasticity Scale (Bohannon and Smith, 1987, Physical Therapy, 67(2), 206-207);
Ouality of Life (QoL) is the perceived quality of an individual’s daily life, that is, an assessment of their well-being or lack thereof;
HAD anxiety and depression is the Hospital Anxiety and Depression Scale;
Forced vital capacity, or FVC, refers to the amount of air that can be forcibly exhaled from the lungs after taking the deepest breath possible.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation, MSC population or compositions to the teachings of the invention without departing from the essential scope thereof.

Therefore, it is intended that the invention not be limited to any particular embodiment contemplated, but that the invention will include all embodiments falling within the scope of the appended claims. The invention will be further illustrated by the following non-limiting Examples.

### BRIEF DESCRIPTION OF THE FIGURES

FIG. 1 is a flowchart illustrating the manufacturing process of isolated, pooled allogeneic MSC population according to the present disclosure.

FIG. 2 shows the results from FACS analysis of an apoptotic marker 7AAD in (A) undiluted drug substance and in (B) diluted drug substance as described in Example 6. Data from 3 separate experiments presented as absolute viability, error bars represent standard deviation.

FIG. 3A, shows overlay of CD200R expression at HMC3 which is treated with IFN_{Y} for 48 hour and FIG. 3B, shows overlay of CD200R expression on HMC3 which is treated with IFN_{Y} and MSC for 48 hours. In both overlay plots, the light grey histogram is from unstained cells and the dark grey histogram is from cell stained with anti-human CD200R.

FIG. 4, panel A, shows overlay of CD183 expression at HMC3 which is treated with IFN_{Y} for 48 hours but panel B, shows overlay of CD183 expression on HMC3 which is treated with IFN_{Y} and MSC for 48 hours. In both overlay plots, the light grey histogram is from unstained cells and the dark grey histogram is from cell stained with anti-human CD183.

FIG. 5, box and whiskers plots demonstrating no significant batch to batch variation with pooling of the WJ-MSC product in terms of relative suppression of PBMC proliferation, prostaglandin E2 secretion and IDO activity.

FIG. 6 shows bar graphs showing that the isolated, pooled allogeneic MSC population according to the present disclosure (TB1) obtained from WJ exhibits higher level of IDO-activity at baseline (unstimulated) compared to MSCs obtained from WJ from single donors (TST-503; TST-526), MSCs obtained from bone marrow (BM-MSC) and JEG-3 cell line (FIG. 6A) and compared to MSCs obtained from WJ from single donors (TST-475; TST-503; TST-526) and the JEG-3 cell line (FIG. 6B).

FIG. 7 is a bar graph showing that the isolated, pooled allogeneic MSC population according to the present disclosure (CB2) obtained from WJ secretes higher levels of PGE2 at baseline (unstimulated) compared to MSCs obtained from WJ from single donors (05-MSC2; 07-MSC3; 09-MSC4 and 11-MSC5).

FIG. 8 is a table showing that the isolated, pooled allogeneic MSC population according to the present disclosure (CB1, CB2 and TB1) do not express the SARS-CoV-2 viral entry receptor, ACE2 receptor or the spike protein priming serine protease TMPRSS2. The housekeeping gene RPL13A was used to confirm relative expression levels and 597 WJMSCs from a single donor are presented as a control.

### EXAMPLES

The present non-limiting Examples describe the generation of the inventive pooled allogeneic MSC composition of in vitro expanded mesenchymal stromal cells, including characterization of cells, selection of appropriate donor derived populations of cells and pooling of said donor derived populations of cells to obtain said composition. Examples 1-5 describe the process of obtaining the inventive pooled allogeneic MSC composition. Examples 6-9 describe a clinical study using said pooled allogeneic MSC composition for treatment and/or prevention of COVID-19 and/or symptoms associated with COVID-19.

As used in the Example section the following terms have the meaning as explained below:

Master Cell Stock - Term used to define Drug Substance Intermediate at certain passage. In the example presented herein, Master Cell Stock is the Drug Substance Intermediate at passage 0. The skilled person will appreciate that the Master Cell Stock may be the Drug Substance Intermediate at passage 1 or 2.

Drug Substance Intermediate - Term used to define MSCs from a single donor that are in production, hence being expanded. Meeting in process quality criteria but has not yet been evaluated with the selection algorithm. Drug Substance Intermediate corresponds to individual donor derived MSC population as disclosed herein, which individual donor derived MSC population has not yet been selected for pooling.

Drug Substance - Term used to define MSCs from a single donor that meet in the manufacturing quality criteria and have been identified as having desired characteristics by the selection algorithm. Hence, not subject to further culturing or expansion. Drug Substance thus corresponds to individual donor derived MSC population as disclosed herein, which individual donor derived MSC population have been selected for pooling.

Drug Product - The term Drug Product refers to a cell suspension of ex vivo expanded Wharton’s jelly derived mesenchymal stem cells (WJMSCs) from multiple donors which have been identified as having desired characteristics by the selection algorithm. Drug Product corresponds to the isolated, pooled allogeneic MSC populations as disclosed herein.

Final Product - The term Final Product refers to a pharmaceutical composition comprising the Drug Product and at least one pharmaceutically acceptable excipient or carrier.

To clarify, the term “antigen X-antibody” and “anti-antigen X-antibody” as used herein both refer to an antibody with affinity for antigen X. Said terms are used interchangeably.

### Example 1

The present Example describes the process of harvesting, transportation, ex vivo expansion, and cryopreservation of MSCs from Wharton’s Jelly. Additionally, maternal blood is tested for infections agents. Furthermore, culture conditions are described.

### Materials and Methods

The manufacturing for the Master Cell Stock of Wharton’s Jelly-derived MSC is a continuous process from the donor qualification and subsequent ex vivo expansion in xeno-free culture system.

Umbilical cord (UC) samples are collected after natural delivery as well as caesarian sections after placenta expulsion and umbilical cord blood collection (for infectious agents screening). Maternal peripheral blood samples are also collected.

For minimizing the risk of contamination, disposable, sterile scissors and forceps are used and fragments of the umbilical cord are placed into sterile transportation containers filled with transportation liquid (99% Sodium Chloride (0.9% sol.) (Fresenius Cat.no: PK03XE050PL) supplemented with 1% Antibiotic/Antimycotic solution (Gibco Cat.no: 15240-062)). Samples are transported inside of protective boxes to the Manufacturer’s Laboratory. Transport conditions are monitored and UC tissue is processed within 72 hours of child delivery. Isolation of Wharton’s Jelly tissue and culture of explants for cell isolation are performed in GMP lab with use of xeno-free, serum-free media and compounds.

Qualification of UC tissue as a source material requires providing complete responses to a medical questionnaire and submission of maternal peripheral blood sample collected within 7 days of the UC collection for infectious agents testing. Donor sampling, testing and screening (medical health questionnaire) is in accordance with Annex II of Directive 2006/17/EC. All donor test kits are validated for intended use. Infectious agent tests performed before umbilical cord qualification are listed in Table 1. Approximately 10-25% of collected samples qualify for further production.

TABLE 1

Infectious agent tests performed from maternal blood (MB) samples.

| | | |
|---|---|---|
| Agent/Disease | Test specification | Results required |
| HIV I, HIV II | anti-HIV I/II | (-) nonreactive |
| HIV I | HIV I Nucleic Acid Test (NAT) | (-) negative |
| HBV | anti-HB core | (-) nonreactive |
| HBV | HBs-Ag | (-) nonreactive |
| HBV | HBV Nucleic Acid Test (NAT) | (-) negative |
| HCV | anti-HCV | (-) nonreactive |
| HCV | HCV Nucleic Acid Test (NAT) | (-) negative |
| CMV | anti-CMV M | (-) nonreactive |
| CMV | anti-CMV G | (-) nonreactive |
| Toxoplasmosis | anti-Toxo M | (-) nonreactive |
| Toxoplasmosis | anti-Toxo G | (-) nonreactive |
| Syphilis | Specific treponemal antibody assay | (-) nonreactive |

*In case of reactive results CMV IgG of maternal blood screening, additional test of Real Time PCR is performed from primary culture. Negative results of RT PCR CMV are required for product release.

Upon arrival at the Manufacturer’s Laboratory, UC fragments are removed from transportation container and washed in a sterile transportation liquid. UC is dissected and blood vessels are removed. Wharton Jelly tissue is minced into 1-2 mm³ scraps with a sterile lancet and placed in xeno-free, serum-free media into culture flask coated with Attachment Solution (1% MSC Attachment Solution Stock 99% D-PBS) for primary explants cultures. Flask are incubated at 37° C. in 5% CO₂. After 1-2 weeks cultures are examined for the presence of adherent, fibroblast-like cells. All non-adherent cells presence in cultures are washed out. The cell culture medium comprises 94% NutriStem® XF (Biological Science, Cat no: 05-200-1A), 5% NutriStem® XF Supplement Mix (Biological Science, Cat no: 05-201-1U) and 1% Antibiotic/Antimycotic solution (Gibco Cat.no: 15240-062). Adherent cells from primary are passaged (controlled enzyme digestion of cultures) upon reaching 90% confluence, generating a master cell stock at P0, and (i) cryopreserved in the presence of cryoprotectant solution (70-80% Human Serum Albumin (5% sol.) (CSL Behring Cat.no: Alburex 5) and 20-30% Dimethyl Sulfoxide (WAK Chemie, Cat.no: WAK-DMSO-50)) for vapor phase of liquid nitrogen storage or (ii) immediately reseeded for further expansion.

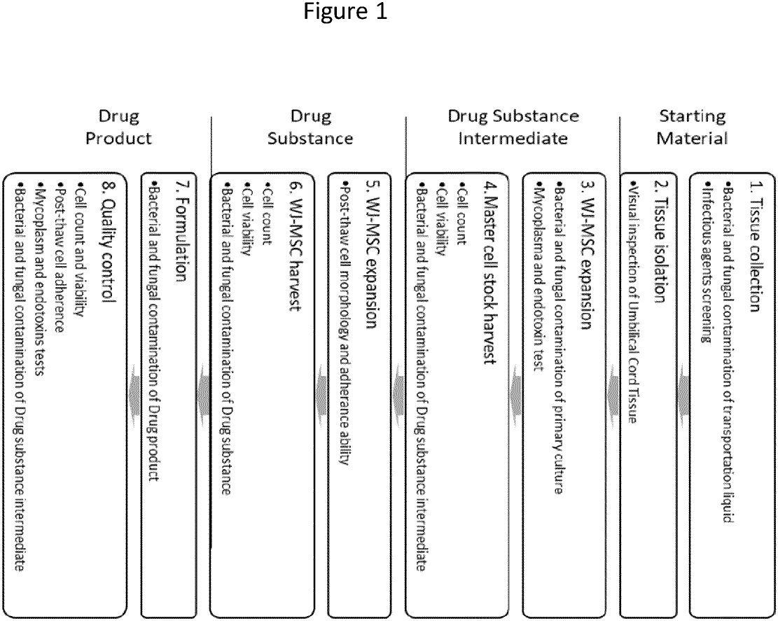

When cells are thawed for expansion at passage 1, the Master Cell Stock exists only for a few hours. Regardless of this short lifespan of the Master Cell Stock, all tests mentioned in FIG. 1 are performed.

TABLE 2

Solutions used in steps of manufacturing process according to the FIG. 1

| Name of solution | Composition | Step of process |
|---|---|---|
| Transportation Liquid | 99% Sodium Chloride (0.9% sol.) 1% Antibiotic/Antimycotic solution | 1 |
| Culture Medium | 94% NutriStem® XF 5% NutriStem® XF Supplement Mix 1% Antibiotic/Antimycotic solution | 2-6 |
| Attachment Solution | 1% MSC Attachment Solution Stock 99% D-PBS | 2-6 |
| Cryoprotectant Solution* | 70-80% Human Serum Albumin (5% sol.) 20-30% Dimethyl Sulfoxide | 7 |

*when Master Cell Stock is intended to be cryopreserved.

There are no animal-origin raw materials used in the manufacturing process from umbilical cord tissue collection until Drug Product release.

During primary cultures of explants, and at the end of manufacturing of Master Cell Stock each cell passage, samples are taken to determine the presence of bacterial and fungal contamination, mycoplasma and endotoxins. The number and viability of cells is evaluated in the Master Cell Stock and Drug Product. Microbial culture, mycoplasma and endotoxins is evaluated from final product. Additionally, one reference sample of Drug Product is can be thawed and testing cell culture is established. This testing culture serve as a source of material for additional final confirmation of product safety purity (by microbial culture and mycoplasma and endotoxins test, karyotype etc.), potency (cell number, adherence efficiency and viability) and identity (cytometric immunophenotyping). Cultures fulfilling the approval criteria listed in Table 3 qualify for next steps of processing or cryopreservation and analytical procedures for evaluation of cultures are explained in Example 2. The quality criteria for impurities is totally less than 5 % of the cells may express any of the negative cell surface markers (analysed collectively) and at least 70 % of the cells have to be positive to for the positive cell surface markers (analyzed separately).

TABLE 3

Tests and acceptance specification

| Test or Specification | Goal | Limits or range |
|---|---|---|
| Appearance | Identity | Single cell suspension, clear to opalescent |
| Sterility of in-process media | safety, purity | bacteria/fungi not detected |
| Sterility of Master Cell Stock | safety, purity | bacteria/fungi not detected |
| Mycoplasma | safety, purity | not detected |
| Endotoxins | safety, purity | not detected (<0.25 IU/ml) |
| Cell count | potency | ≥ 500 × 10⁶ |
| Cell viability | potency | ≥ 80% |
| Surface adherence | potency, identity | ≥ 90% of adherent cells |
| Karyotype | safety | 46 and sex chromosomes |
| CD45 antigen expression | identity | < 5% |
| CD34 antigen expression | identity | < 5% |
| CD14 antigen expression | identity | < 5% |
| CD19 antigen expression | identity | < 5% |
| CD3 antigen expression | identity | < 5% |
| CD73 antigen expression | identity | ≥ 70% |
| HLA-DR antigen expression | identity | < 5% |
| CD90 antigen expression | identity | ≥ 70% |
| CD105 antigen expression | identity | ≥ 70% |
| Osteogenesis assay | identity | Detection of osteocytes |
| Adipogenesis assay | identity | Detection of adipocytes |
| Chondrogenesis assay | identity | Detection of chondrocytes |
| Excipient | | |
| 0.9 % sodium chloride | | |
| Human Serum Albumin | | 5 % ± 0.5 volume |
| DMSO | | 10 % ± 1% volume |

### Results

Manufacturer demonstrated the microbiological safety of WJMSC derived from umbilical cords obtained after natural deliveries. The addition of antibiotic/antimycotic solution to the transportation liquid and during next steps of manufacturing resulted in the absence of microorganism (bacteria and fungi) in Master Cell Stock as well as in Drug Product.

The retrospective analysis the characteristic of cells obtained from different donors allow to get criteria to be met by all MSCs for manufacturing comparable Drug Products as is referred in Table 4.

The present procedures provide highly uniform MSC populations which fulfill the required safety criteria.

TABLE 4

Analysis of different batches of Master Cell Stock
Batch #

| Test | TST 626 | TST 668 | TST 664 | TST 681 | TST 642 |
|---|---|---|---|---|---|
| Inf. agents | neg | neg | neg | neg | neg |
| Sterility | neg | neg | neg | neg | neg |
| Mycoplasma | neg | neg | neg | neg | neg |
| Endotoxins | neg | neg | neg | neg | neg |
| Viability (%) | 98.82% | 97.39% | 99.06% | 98.5% | 98.8% |
| CD45^{∗∗} | 0.04% | 0.33% | 0.00% | 0.02% | 0.03% |
| CD34^{∗∗} | 0.04% | 0.33% | 0.00% | 0.02% | 0.03% |
| CD14^{∗∗} | 0.04% | 0.33% | 0.00% | 0.02% | 0.03% |
| CD19^{∗∗} | 0.04% | 0.33% | 0.00% | 0.02% | 0.03% |
| HLA-DR^{∗∗} | 0.04% | 0.33% | 0.00% | 0.02% | 0.03% |
| CD73 | 85.19% | 81.02% | 86.66% | 92.76% | 93.9% |
| CD90 | 95.82% | 99.86% | 99.48% | 99.97% | 98.39% |
| CD105 | 95.56% | 86.47% | 98.79% | 98.66% | 96.72% |

^{∗}Positive results of CMV IgG from maternal blood. Real time PCR test from primary culture has confirmed the lack of viral DNA in culture cells
^{∗∗}Tests performed in one antibody panel

### Example 2

The present Example describes characterization of MSCs from donors based on morphology, proliferative capacity and expression of markers for MSC according to the criteria of the ISCT. Furthermore, the cells are screened for the presence of mycoplasma, endotoxins, bacterial contaminants, fungal contaminants, viral contaminants and/or endotoxins and karyotype testing is performed. The described characterization results in identification of MSC populations derived from Drug Substance Intermediates, which MSCs fulfill quality criteria for pooling.

### Materials and Methods

First, MSCs must be plastic-adherent when maintained in standard culture conditions. Only plastic adherent cells are subject to the analytical procedures described below. Cultures are screened according to the analytical procedures given below.

### Analytical Procedures

### Infectious Agents

Sampling. The source material for WJ-MSC manufacturing (placental part of the umbilical cord) is obtained within several minutes after placenta expulsion. That is why the only way of infectious agent transmission is from maternal blood via placenta. Two samples of donor-mother’s peripheral blood are collected at the day of delivery and source tissue harvest.

Analysis. ABBOTT ARCHITECT 2000 for chemiluminescent immunoassay and Procleix PANTHER System for NAT assay are used according to manufacturer’s instructions. The following test using the Abbott ACHITECT for chemiluminescent immunoassay are performed HIV Ag/Ab Combo; HBsAg Qualitative II; Anti-HBc II; Anti-HCV; CMV IgM; CMV IgG; Toxo IgM; Toxo IgG; and Syphilis TP. Additionally, Proclex Utrio Plus Assay is used to qualitative screen in vitro nucleic acid amplification for HIV-1 RNA, hepatitis C virus (HCV) RNA and hepatitis B virus (HBV) DNA in plasma and serum specimens from human donors.

Acceptance criteria. Results of test must be “negative”, “non-reactive” or “not detected” for infectious agents (except CMV IgG: having positive results of this test Manufacturer performs confirm the lack of CMV DNA in the product by RealTime PCR test).

### Sterility

Sampling. A sample (10 mL) of cells and supernatant from cultures after enzymatic harvest.

Analysis. Sample is seeded into two BACTEC bottles intended for growth of anaerobic and aerobic bacteria as well as for detection of fungal contamination. Bottles are placed in BACTEC FX400 microbial analyzer for 14 days.

Acceptance criteria. Results of test must be “negative” or “not detected” for aerobic anaerobic bacteria as well as for fungal microorganisms after 14 days incubation.

### Mycoplasma.

Sampling. A sample (0.1 mL) of cells and supernatant from cultures after enzymatic harvest

Analysis. The Venor®GeM Classic Assay (Merck KGaA, cat no MP0025) is based on PCR amplification is uses according to the manufacturer’s instructions.

Acceptance criteria. Results of test must be “not detected” for amplification product in the gel slot.

### Endotoxin.

Sampling. A sample (0.5 mL) of cells and supernatant from cultures after enzymatic harvest

Analysis. The Endosafe®-PTS™, (Charles River Laboratories, cat no PTS2005F) realtime endotoxin testing system, is used according to the manufacturer’s recommendations.

Acceptance criteria. Results of test must be “not detected”

### Cell Count.

Sampling. A sample (0.5 mL) of cells from cultures after enzymatic harvest Analysis. Light microscopic analysis of cell number with use of Malassez’ chamber.

Acceptance criteria. Not less than 98% of required number of cells

### Cell Viability.

Sampling. A sample (0.5 mL) of cells from cultures after enzymatic harvest

Analysis. Flow cytometric analysis of 7-AAD (Becton, Dickinson and Company, Cat.no. 559925) stained cell suspension is performed using a FACS Calibur flow cytometer. Acceptance criteria. More than 80% of viable cells.

### Immunophenotyping.

Sampling. A sample (0.5 mL) of cells from cultures after enzymatic harvest

Analysis. Flow cytometric analysis of cells previously labelled with monoclonal antibodies is performed using a FACS Calibur flow cytometer Antigens tested a listed in Table 4.

Acceptance criteria- Expression of CD73, CD90 and CD105 on more than 70% of cells. Lack of expression of lineage antigens (CD45, CD34, CD14 or CD11b, CD79alpha or CD19 and HLA-DR surface molecules).

### Karyology.

Sampling. Cell culture performed especially for this assay.

Analysis. Culture is blocked with Colcemid and stained with Giemsa. The number of chromosomes and structural aberrations are evaluated.

Acceptance criteria. 46 chromosomes, XY or XX; no visible aberrations.

### Differentiation Assay

### Cells Are Subject to Differentiation

Analysis. Differentiation assays are used according to manufacturer’s instructions. Human Mesenchymal Stem Cell Functional Identification Kit, Catalog Number SC006, R&D Systems, Inc. designed for the identification of human MSCs based on their ability to differentiate into multiple mesenchymal lineages. This kit contains specially formulated adipogenesis, chondrogenesis, and osteogenesis media supplements, which can be used to effectively differentiate MSCs into adipogenic, chondrogenic, or osteogenic lineages. A panel of antibodies, consisting of anti-mFABP4, anti-hAggrecan, and anti-hOsteocalcin, are included to define the mature phenotypes of adipocytes, chondrocytes, and osteocytes, respectively. StemPro® Chondrogenesis Differentiation Kit, Catalogue number: A1007101, Thermo Fisher Scientific Inc. developed for the chondrogenic differentiation of mesenchymal stem cells (MSCs) in tissue-culture vessels. The kit contains all reagents required for inducing MSCs to be committed to the chondrogenesis pathway and generate chondrocytes.

Acceptance criteria. Ability to differentiate to osteoblasts, adipocytes and chondroblasts in vitro.

### Results

Obtained MSC populations are plastic-adherent when maintained in standard culture conditions. The MSC express CD105, CD73 and CD90, and lack expression of CD45, CD34, CD14 or CD11b, CD79alpha or CD19 and HLA-DR surface molecules as given in Example 3 and are able to differentiate to osteoblasts, adipocytes and chondroblasts in vitro. Thus, MSC populations eligible for pooling are identified.

While these criteria are currently employed, they may require modification as new knowledge unfolds leading to for example alteration of the definition of MSC according to the criteria of the ISCT), the present inventors believe the above minimal set of standard criteria will foster a more uniform characterization of MSC. As used herein, MSCs are defined according to criteria from ISCT.

### Example 3

The present Example describes the screening assays used to characterize the said MSC populations derived from Drug Substance intermediates for morphological, proliferative and functional characteristics in order to select the MSC populations to be pooled.

### Materials and Methods

Below follows the description of 6 assays used to characterize the MSC populations.

Assay 1 - IDO: IDO assay is used to analyze the immunosuppressive capacity of Drug Substance Intermediate or Drug Substance, i.e. mesenchymal stem/stromal cells (MSC).

The UC-MSC immunomodulatory potential is reported as a measure of indoleamine 2,3-dioxygenase (IDO) activity, determined by measuring tryptophan and kynurenine in the culture supernatant. Indoleamine-pyrrole 2, 3-dioxygenase (IDO or INDO EC 1.13.11.52) is a heme-containing enzyme that in humans is encoded by the IDO1 gene. The IDO enzyme converts L-tryptophan to N-formylkynurenine (or kynurenine), an immunosuppressive molecule that acts as an inhibitor of immune cell proliferation - including T cells, as well as exhibiting antibacterial and antiviral functions. The IDO activity is the ratio of kynurenine/tryptophan and can be determined by calculating the amount of tryptophan and kynurenine present in cell culture supernatants using an ELISA kit. When stimulated with interferon gamma (IFN_{Y}) in the presence or absence of tumor necrosis factor alpha, mesenchymal stem/stromal cells (MSC) secrete more IDO than when they are unstimulated.

Inducible IDO activity indicates that the cells released have functional potency, related to immunomodulation.

MSC culturing: Seed 10 000 MSC / well in 48-well cell culture plates in 100 µl assay medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)). Dilute IFN_{Y} from stock, 1 mg/ml (ThermoFisher Scientific, cat no. PHC4033). The final concentration of IFN_{Y} / well is 100 ng/ml. Add 100 µl of 200 ng/ml IFN_{Y} to the wells. Add 100 µl assay medium to non-stimulated cells (no IFN_{Y}). Incubate cell culture plate at 37° C., 5% CO₂ for 72 hours. Remove the supernatant from each well and store in micro tubes at -20° C. until further processing for ELISA analysis.

Tryptophan and kynurenine measurements are done according to manuals provided by the ELISA-kit manufacturer (Immundiagnostik AG, cat no. K 3730 and K 3728). Both tryptophan and kynurenine ELISA are performed on the same day but at separate occasions. The two ELISAs are conducted according to manufacturer’s instructions; see the manuals for respective ELISA.

Absorption at 450 nm with background subtraction at 620 nm is measured in a Spectramax microplate reader (Molecular Devices, Spectramax 190).

Analyzing results: Amount of absorbance measured is inversely proportional to the amount of amino acid present in the sample; i.e. the lower the OD450, the more kynurenine or tryptophan there is. The 4PL-algorithm (Four Parameter Logistic Regression) is used to calculate results (software SoftMax Pro 7.0.2, Molecular Devices), as recommended by kit manufacturer. Concentrations are determined directly from the standard curve. The control samples provided with the kits should are evaluated for acceptability: if outside the acceptable range according to the manufacturer of the kit, the samples need to be re-assayed.

### Results

Relative IDO bioactivity of IFNy treated cells from Drug Substance Intermediates are used for ranking of the samples according to the selection algorithm (Example 4). The donors with the highest increase in bioactivity get the highest ranking score. The ranking score (Table 5) is later used in the final selection of donor (see Example 4).

TABLE 5

Illustrative example of ranking score based on IDO fold increase.

| Donor | IDO fold increase | Ranking score | Donor | IDO fold increase | Ranking score |
|---|---|---|---|---|---|
| D5 | 150 | 3 | D3 | 112 | 2 |
| D7 | 130 | 3 | D2 | 100 | 1 |
| D4 | 125 | 3 | D8 | 90 | 1 |
| D6 | 124 | 2 | D10 | 85 | 0 |
| D1 | 112 | 2 | D9 | 80 | 0 |

### Assay 2: Proliferation Assay

This method is used to quantitatively measure the immunosuppressing effect of the Drug Substance Intermediate and/or Drug Substance, i.e. umbilical cord derived MSCs have on the proliferation of peripheral blood mononuclear cells (PBMC). MSC have been shown to suppress T-lymphocyte proliferation. Mixed lymphocyte reactions with MSC are frequently used to demonstrate the immunosuppressive activity of MSC.

Phytohaemagglutinin (PHA) is used as a mitogen which activates proliferation of T-lymphocytes. The immunosuppressive activity of Drug Substance Intermediate and/or Drug Substance is quantified as the decrease in proliferation of PHA stimulated T-lymphocytes.

Culturing and CFSE priming: MSC (2× 10⁵ cells/well) in 500 µl of working medium (RPMl1640 (ThermoFisher Scientific, cat no. 12633012) + 2 mM Glutamax (ThermoFisher Scientific, cat no. 35050061) + 100 U/ml Pest (ThermoFisher Scientific, cat no. 15140122) + 10% FBS (ThermoFisher Scientific, cat no. 16140071) are seeded in 12-well cell culture plates. The plates are incubated at 37° C., 5% CO₂ for 2 hours for plastic adherence of cells. Lymphoprep™ kit is used for isolation of mononuclear cells from donated peripheral blood, retrieved from the blood central, according to manufacturer’s instructions (Stem Cell Technologies, cat no. 07801). PBMC are suspended in RPMI 1640, 22 × 10⁶ cells/ml. CellTrace™ CFSE Cell Proliferation Kit (ThermoFisher Scientific, cat no. C34554) is used for analysing the proliferation according to manufacturer’s instruction. CFSE-primed PBMC (1×10⁶ cells /well) are seeded to the 12-well cell culture plate and PHA is added as a mitogen.

Analysis: CFSE positive cells are analyzed by Accuri C6 plus flow cytometer. CFSE histogram includes three or four peaks and the first top from the right represents undivided cells (G0). The following tops show different generations (G1-G4). Proliferation Index (PI) is calculated as the total number of cells of all generations divided by the number of parent cells that entered cell division.

TABLE 6

Illustrative example of ranking score based on proliferation index

| Donor | Proliferation Index | Ranking score | Donor | Proliferation Index | Ranking score |
|---|---|---|---|---|---|
| D7 | 1.02 | 3 | D1 | 1.14 | 2 |
| D5 | 1.06 | 3 | D8 | 1.16 | 1 |
| D6 | 1.07 | 3 | D2 | 1.17 | 1 |
| D4 | 1.11 | 2 | D9 | 1.22 | 0 |
| D3 | 1.13 | 2 | D10 | 1.28 | 0 |

### Results

The average proliferation index for each Drug Substance Intermediates is used for relative comparison of the donors. The donors with the lowest PI get the highest ranking score. The ranking score (Table 6) is later used in the final selection of donor (see Example 4).

### Assay 3: Prostaglandin E2

Prostaglandin E2 (PGE2) assay evaluates Drug Substance Intermediate and/or Drug Substance secretion of PGE2 when co-cultured with peripheral blood mononuclear cells (PBMCs) activated with Phytohemagglutinin (PHA).

Cell culturing: Cells are cultured in assay medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) for 3 days in co-culture cell ratio MSC-PBMC 1:5, in the presence and absence of PHA (Merck, cat no. 11082132001). 40 000 MSCs are seeded per well in 12-well cell culture plates. Cell culture plates are incubated at 37° C., 5% CO₂ for 2 h to allow the cells to adhere.

Lymphoprep™ kit is used for isolation of mononuclear cells from donated peripheral blood, retrieved from the blood central, according to manufacturer’s instructions (Stem Cell Technologies, cat no. 07801). PBMC are suspended in assay medium, 0.5 × 10⁶ cells/ml, and 400 µl is seeded into wells intended for PBMC. 500 µl assay medium is added to wells without PBMC. 100 µl/well of 100 µg/ml is added to PHA to PBMC containing wells and the cell culture plate is incubated at 37° C., 5% CO₂ for 72 hours. The supernatant is removed from each well and centrifuged for 5 min at 500 g to remove particulates. The supernatant is frozen and stored at -20° C. until further processing for ELISA analysis.

The Parameter™ Prostaglandin E2 Immunoassay kit is used for PGE2 expression detection according to manufacturer’s instruction (Bio-Techne, cat no. KGE004B) and is analyzed with Spectramax microplate reader (Molecular Devices, Spectramax 190). The 4PL-algorithm (Four Parameter Logistic Regression) is used to calculate results (software SoftMax Pro 7.0.2, Molecular Devices).

### Results

The average expression of PGE2 in pg/ml for each Drug Substance Intermediate is used for relative comparison of the donors. The donors with the highest expression level get the highest ranking score. The ranking score (Table 7) is later used in the final selection of donors (see Example 4).

TABLE 7

Illustrative example of ranking score based on PGE2 expression

| Donor | PGE2 expression | Ranking score | Donor | PGE2 expression | Ranking score |
|---|---|---|---|---|---|
| D5 | 14900 | 3 | D3 | 12400 | 2 |
| D7 | 13000 | 3 | D2 | 12000 | 1 |
| D4 | 12900 | 3 | D8 | 11100 | 1 |
| D6 | 12600 | 2 | D10 | 10000 | 0 |
| D1 | 12500 | 2 | D9 | 9000 | 0 |

### Assay 4: HLA-G

The HLA-G assay evaluates Drug Substance Intermediate and/or Drug Substance expression of soluble and/or intracellular HLA-G in response to IFNy, IL-10 and/or PHA.

Cell culture: 50 000 MSC and 25 000 JEG-3 cells (positive control cells, ATCC, cat no. ATCC^{®} HTB-36^{TM} ) are seeded per well in 12-well cell culture plates in 1 ml assay medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) with a final concentration of 10-50 ng/ml IL-10 (Miltenyi Biotec, cat no. 130-108-985) or 25-100 ng/ml IFN_{Y} (ThermoFisher Scientific, cat no. PHC4033) or without stimulation. Cells are incubated at 37° C., 5% CO₂ for 48 to 72 h. The supernatant is removed from each well and stored at -20° C. for ELISA analysis of soluble HLA-G.

Intracellular HLA-G: The adherent cells are washed twice with DPBS and detached with TrypLE Express (Thermo Scientific, cat no. 12604021). The BD Cytofix/Cytoperm™ is used for fixation and permeabilization of cells according to manufacturer’s instruction (Becton, Dickinson and Company, cat no. 554714). Cells are stained with HLA-G (PE) antibody (EXBIO Praha, cat no. 1P-431-C100) according to manufacturer’s instruction and analyzed by flow cytometry (Merck, Guava easyCyte 5HT).

Soluble HLA-G: The concentration of HLA-G in the supernatant is analyzed with sHLA-G ELISA kit (Enzo Life Sciences, cat no. ALX-850-309-KI01) according to manufacturer’s instruction.

### Results

The Drug Substance Intermediates and/or Drug Substances are analyzed for both intracellular and soluble HLA-G expression and receive a score based on the relative expression compared to with the other samples analyzed. The total score from intracellular and soluble HLA-G expression is summarized and the Drug Substance Intermediates receive a ranking score (Table 8) that is used for the final selection of donors (see Example 4).

TABLE 8

Illustrative example of ranking score based on sHLA-G and iHLA-G scores.

| Donor | Soluble HLA-G | sHLA-G score | Intracellular HLA-G | iHLA-G score | HLA-G score | Ranking score |
|---|---|---|---|---|---|---|
| D5 | 1110 | 8 | 0% | 9 | 17 | 3 |
| D7 | 1100 | 7 | 1% | 6 | 13 | 3 |
| D4 | 990 | 4 | 0% | 9 | 13 | 3 |
| D6 | 1200 | 9 | 6% | 1 | 10 | 2 |
| D1 | 1002 | 6 | 1% | 6 | 12 | 2 |
| D3 | 980 | 3 | 0% | 9 | 12 | 2 |
| D2 | 1000 | 5 | 8% | 0 | 5 | 1 |
| D8 | 937 | 2 | 5% | 3 | 5 | 1 |
| D10 | 825 | 1 | 5% | 3 | 4 | 0 |
| D9 | 600 | 0 | 2% | 4 | 4 | 0 |

### Assay 5: Morphology

Cell morphology of the Drug Substance Intermediate and/or Drug Substance cultures are continuously surveilled. Cells are being visually inspected during expansion as well as before harvesting and evaluated based on:

| | |
|---|---|
| • Size of cell | normal/big |
| • Size of nuclei | normal/big |
| • Shape of cell | normal/abnormal |
| • Ratio between cell and nuclei size | normal/abnormal |

### Results

Drug Substance Intermediate cells are visually assessed based on the criteria above. Only samples with more than 90% normal cells are accepted. The frequency of abnormal cells is used for ranking (Table 9) the Drug Substance Intermediates (see Example 4).

TABLE 9

Illustrative example of ranking score based on morphology.

| Donor | Percentage abnormal cells | Ranking score | Donor | Percentage abnormal cells | Ranking score |
|---|---|---|---|---|---|
| D5 | 0% | 3 | D4 | 2% | 2 |
| D6 | 0% | 3 | D3 | 5% | 1 |
| D7 | 0% | 3 | D8 | 5% | 1 |
| D1 | 1% | 2 | D9 | 6% | 0 |
| D2 | 1% | 2 | D10 | 8% | 0 |

### Assay 6: Fluorospot

Drug Substance Intermediate and/or Drug Substance are cultured in Fluorospot specific 96 well plates pre-coated with antibodies (service provided by MabTech). 1000 - 3000 cells are seeded per well in 100 µl assay medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) and incubated for 48 hours in absence or presence of stimuli. Stimulations used are Poly I:C (Invivogen, cat no. tlrl-pic), r848 (Invivogen, cat no. tlrl-r848), GABA (Diamyd Medical) and IFNy (ThermoFisher Scientific, cat no. PHC4033). The expression of IL-2, IL-4, IL-6, IL-8, IL-12, IL-12/13, IL-17A IL-21, IL-22, IL-29, IL-31, TGFβ1, GM-CFS, IFNa, IFNɣ, apoE and TNFa is analyzed by Fluorospot (MabTech). Earlier batches of the pooled allogeneic MSC composition, i.e. Drug Product, are used as reference. The assay contains both proteins and cytokines considered desirable and undesirable. For example, it is considered positive if the cells are expressing IL-6 but negative if they express IFNy.

TABLE 10

Antibodies used in said Fluorospot-assay.

| | | | |
|---|---|---|---|
| Fluorospot-assay | Detection antibody | Fluorospot-assay | Detection antibody |
| IL-2 | human mAb MT8G10-biotin, 0.5 mg/ml | IL-29 | human mAb MT6G4-biotin, 0.5 mg/ml |
| IL-4 | human mAb IL-4 II-biotin, 1 mg/ml | IL-31 | human mAb MT158-biotin, 0.5 mg/ml |
| IL-6 | human mAb 39C3-biotin, 1 mg/ml | IFNα (pan) | human mAbs MT2/4/6-biotin, 1 mg/ml |
| IL-8 | human mAb MT8F19-biotin, 0.5 mg/ml | IFNɣ | human mAb 7-B6-1-biotin, 1 mg/ml |
| IL-13 | human mAb IL13-3-biotin, 0.5 mg/ml | TNFα | human mAb TNF5-biotin, 0.5 mg/ml |
| IL-21 | human mAb MT21.3 m-biotin, 0.5 mg/ml | GM-CSF | human mAb 23B6-biotin, 1 mg/ml |
| IL-22 | human mAb MT7B27-biotin, 0.5 mg/ml | TGFβ1 (latent form) | human mAb MT517-biotin, 0.5 mg/ml |

### Results

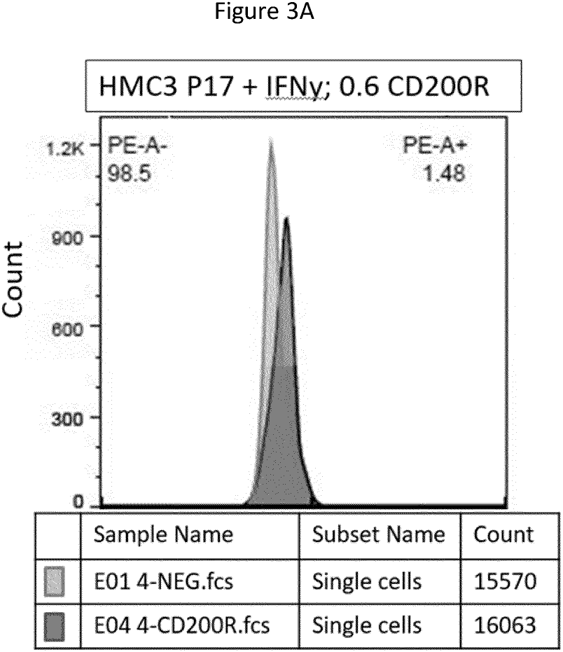

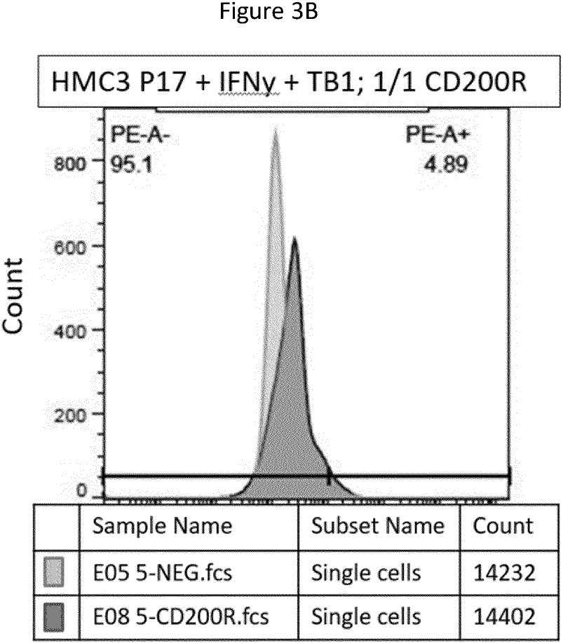

The results are analyzed with the software provided with the Fluorspot reader. The program generates both visual and numeric output (see FIG. 3).

The Drug Substance Intermediate samples are scored in relation to the reference sample (numeric value). A threshold value for positive vs. negative is predefined for each marker and the Drug Substance Intermediates are scored according to Table 12.

TABLE 11

Marker translation to numeric scores

| Type of marker | Result | Numeric score | Type of marker | Result | Numeric score |
|---|---|---|---|---|---|
| Positive markers | Negative | 0 | Negative markers | Negative | 0 |
| | Positive | 1 | | Positive | -2 |
| | Higher than reference | 2 | | | |

The final ranking of the Drug Substance Intermediates is based on the summarized score from all markers analyzed with and/or without stimuli (Table 12).

TABLE 12

Illustrative example of ranking score for all markers analyzed.

| Donor | M1 | M1 S1 | M2 | M3 | M4 S1 | M4 S2 | M4 S3 | Summarized score |
|---|---|---|---|---|---|---|---|---|
| D1 | 1 | 1 | 0 | 0 | 1 | 1 | 1 | 5 |
| D2 | 1 | 1 | 0 | 0 | 1 | 1 | 1 | 5 |
| D3 | 1 | 1 | -1 | 0 | 1 | 1 | 1 | 4 |
| D4 | 1 | 1 | 0 | 0 | 1 | 1 | 1 | 5 |
| D5 | 2 | 1 | 0 | 0 | 2 | 1 | 1 | 7 |
| D6 | 2 | 2 | 0 | 0 | 1 | 2 | 1 | 8 |
| D7 | 1 | 2 | 0 | 0 | 2 | 1 | 1 | 7 |
| D8 | 0 | 0 | 0 | 0 | 1 | 1 | 1 | 3 |
| D9 | 0 | 0 | 0 | 0 | 0 | 0 | 1 | 1 |
| D10 | 1 | 1 | 0 | 0 | 0 | 0 | 1 | 3 |

D1 = donor 1, D2 = donor 2 etc. M1 = marker 1, M2 = marker 2 etc. S1 = stimuli 1, S2 = stimuli 2 etc. M1 and M4 are positive markers. M2 and M3 are negative markers.

The ranking of the Drug Substance Intermediates is based on the score which generates are ranking score for the Fluorospot assay. The ranking score is later used for the overall selection of donors described in Example 4. The Drug Substance Intermediate sample with the highest score will also get the highest ranking score (see Table 13). Furthermore, it is also possible to use some or all of the Fluorospot results as input in the selection algorithm, i.e. data from each analyzed protein as a separate component in the selection algorithm.

TABLE 13

Illustrative example of ranking score based on Summarized score from the Fluorospot assay

| Donor | Score | Ranking score | Donor | Score | Ranking score |
|---|---|---|---|---|---|
| D6 | 8 | 3 | D4 | 5 | 2 |
| D5 | 7 | 3 | D3 | 4 | 1 |
| D7 | 7 | 3 | D8 | 3 | 1 |
| D1 | 5 | 2 | D9 | 3 | 0 |
| D2 | 5 | 2 | D10 | 1 | 0 |

### Assay 7: Microglia Proliferation Assay

This method is used to quantitatively measure the immunosuppressing effect of the Drug Substance Intermediate and/or Drug Substance, i.e. MSCs as described herein, have on the proliferation of microglia cells. MSC have been shown to suppress microglia proliferation. Co-culture of microglia and MSC is used to demonstrate the immunosuppressive activity of MSC. Lipopolysaccharide (LPS) is used as a mitogen which activates proliferation of microglia. The immunosuppressive activity of Drug Substance Intermediate and/or Drug Substance is quantified as the decrease in proliferation of LPS stimulated microglia cells.

Co-culturing CFSE-primed microglia with MSC: Microglia cells (1 × 10⁶ cells/ml) are suspended in DPBS+2%FBS and stained with CellTrace ™ CFSE Cell Proliferation Kit (ThermoFisher Scientific, cat no. C34554) according to manufacturer’s instruction. MSC (5000 cells/well) in 100 µl of working medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) are seeded in 48-well cell culture plates. 200 µl of CFSE-primed microglia cells (25000 cells/well) in DMEM+10%FBS are seeded in 48-well cell culture plate simultaneously. After 24 hours at 37° C., 5%CO₂, LPS (from E.coli, Sigma Aldrich Cat No;10900010L4391) with a final concentration of 1 pg/ml is added and the 48-well cell culture plates are incubated for a further 48 hours, then the medium is removed and the adherent cells are washed twice with DPBS and detached with 25 µl TrypLE Express (Thermo Scientific, cat no. 12604021).1 ml working medium is added to the wells and cells are transferred to tubes and centrifuged at 300 g for 5 min. The supernatants are removed and cells are re-suspended in 200 µl DPBS+2%FBS and run on Accuri C6 Plus Flow cytometer.

Analysis: Total cell amount of each sample is calculated by multiplying cells/µl of CFSE stained cells x 200 µl. Growth index is calculated by dividing the total cells after 72 hour to cell amount at starting of assay.

### Results

The average growth index for each Drug Substance Intermediate is used for relative comparison of the donors. The donors with the lowest GI get the highest ranking score. The ranking score is later used in the final selection of donors (see Example 5).

TABLE 14

Illustrative example of ranking score based on proliferation index.

| Donor | Growth Index | Ranking score | Donor | Growth index | Ranking score |
|---|---|---|---|---|---|
| D7 | 1.92 | 3 | D1 | 2.35 | 2 |
| D5 | 1.95 | 3 | D8 | 2.36 | 1 |
| D6 | 2.01 | 3 | D2 | 2.44 | 1 |
| D4 | 2.11 | 2 | D9 | 2.48 | 0 |
| D3 | 2.22 | 2 | D10 | 2.70 | 0 |
| RefTB1 | 2.25 | | Negative | 3.50 | 0 |

### Assay 8: Microglia CD183 Expression

Chemokine receptor CXCR3 is a receptor in the CXC chemokine receptor family. Other names for CXCR3 are G protein-coupled receptor 9 (GPR9) and CD183. CXCR3 is expressed primarily on activated T lymphocytes and NK cells and some epithelial cells as well as on microglia cells.

Co-culturing microglia cells with MSC: Microglia cells are re-suspended in serum-free media and seeded in CellBIND culture flasks (1 × 10⁶ cells/T75). After 2 hours at 37° C., 5% CO₂, MSC (0.2 × 10⁶ cells/T75) and IFN_{Y} (10 ng/ml) are added to the microglia cells. The ratio will be 5:1 microglia cells: MSC. The cells are incubated for 48 h before washing with DPBS and detaching the cells with 500 µl TrypLE Express (Thermo Scientific, cat no. 12604021). Serum free medium is added and cells are transferred to tubes and centrifuged at 200 g for 5 min. The supernatant is removed and 3 ml working medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) is added. Cells are counted and divided equally to flow cytometry staining tubes. Cells are stained with 16 µl Anti Human CD183 from BD Pharmingen (Product no; 557185; PE mouse Anti human CD183) for 30 min at RT protected from light. Staining is stopped by adding 2 ml DPBS+2%FBS to each tube. Cells are centrifuged at 200 g for 5 min, supernatants are discarded and cell pellets are re-suspended in 200 µl DPBS+2%FBS. 150 µl of each sample is used for running flow cytometry. A minimum of 30000 events is recorded.

### Results

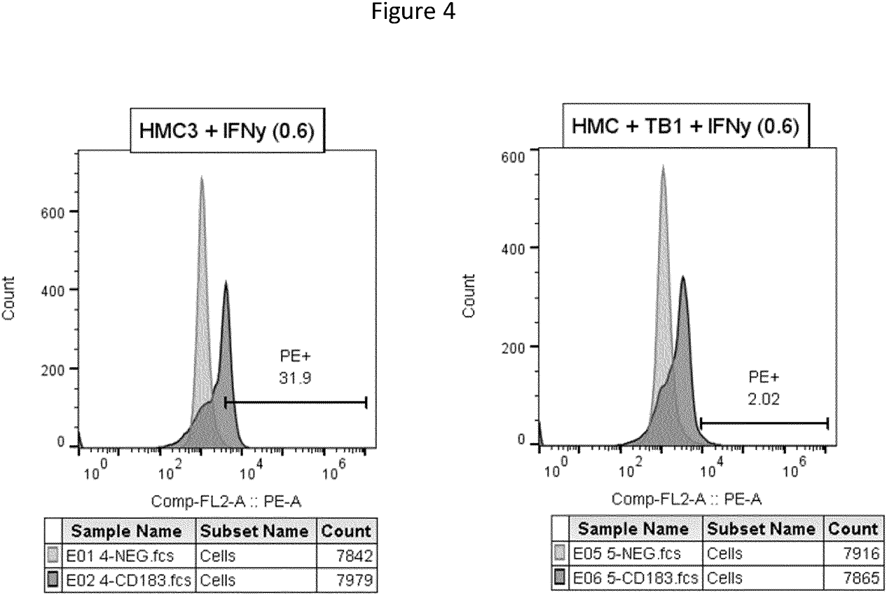

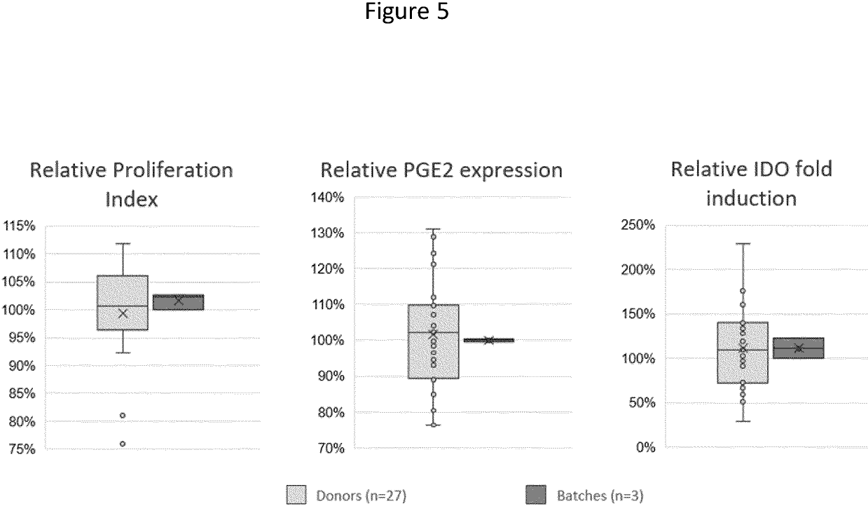

The FACS results are analyzed with Flow-Jo software. The inactivation of microglia is calculated as the decrease of CD183 positive microglia cells caused by the Drug Substance Intermediates (FIG. 4A), calculated as:

$$S u p p r e s s i o n \% = 1 - \frac{\% C D 183 p o s i t i v e m i c r o g l i a c e l l s w i t h M S C}{\% C D 183 p o s i t i v e m i c r o g l i a c e l l s}$$

The donors with the highest suppression percent get the highest ranking score. The ranking score is later used in the final selection of donors.

TABLE 15

Illustrative example of ranking score based on CD183 suppression.

| Donor | Suppression | Ranking score | Donor | Suppression | Ranking score |
|---|---|---|---|---|---|
| D5 | 92% | 3 | D8 | 80% | 2 |
| D6 | 89% | 3 | D3 | 75% | 1 |
| D7 | 88% | 3 | D4 | 55% | 1 |
| D1 | 84% | 2 | D9 | 32% | 0 |
| D2 | 82% | 2 | D10 | 28% | 0 |
| RefTB1 | 82% | 2 | Negative | 0% | 0 |

### Assay 9: Microglia CD200R Expression

The CD200 transmembrane glycoprotein, mostly expressed in neurons, interacts with its receptor, CD200R which is expressed in the CNS almost exclusively in microglia as well as in other CNS macrophages, to inhibit microglial priming and holds microglia in a quiescent state. Fold increase of CD200R expression on microglia cells by MSC is analyzed to measure immunosuppression or the shift towards an M2 phenotype. Co-culturing microglia cells with MSC: Microglia cells are re-suspended in serum-free media and seeded in CellBIND culture flasks (0.6 × 10⁶ cells/T75). After 2 hours at 37° C., 5% CO₂, MSC (0.6 × 10⁶ cells/T75) and IFN_{Y} (10 ng/ml) are added to the microglia. The ratio will be 1:1 microglia cells: MSC. The cells are cultured for 48 h before washing with DPBS and detaching the cells with 500 µl TrypLE Express (Thermo Scientific, cat no. 12604021). Serum free medium is added and cells are transferred to tubes and centrifuged at 200 g for 5 min. The supernatant is removed and 3 ml working medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) is added. Cells are counted and divided equally to flow cytometry staining tubes. Cells are stained with 10 µl Anti Human CD200R from Abcam (Product no; ab33366; PE mouse Anti human CD200R) for 30 min at RT protected from light. Staining is stopped by adding 2 ml DPBS+2%FBS to each tube. Cells are centrifuged at 200 g for 5 min, supernatants are discarded and cell pellets are re-suspended in 200 µl DPBS+2%FBS. 150 µl of each sample is used for running flow cytometry. A minimum of 30000 events are recorded.

### Results

The FACS results are analyzed with Flow-Jo software. The inactivation of microglia is calculated as the fold increase of CD200R positive microglia cells caused by the Drug Substance Intermediates (FIG. 3), calculated as:

$$\begin{matrix}F o l d i n c r e a s e = \\ \frac{\% C D 200 R p o s i t i v e m i c r o g l i a c e l l s w i t h M S C}{\% C D 200 R p o s i t i v e m i c r o g l i a c e l l s} - 1\end{matrix}$$

The donors with the highest CD200R fold increase get the highest ranking score. The ranking score is later used in the final selection of donors.

TABLE 16

Illustrative example of ranking score based on CD200R fold increase.

| Donor | Fold increase | Ranking score | Donor | Fold increase | Ranking score |
|---|---|---|---|---|---|
| D5 | 3 | 3 | D4 | 2.08 | 2 |
| D1 | 2.96 | 3 | D3 | 1.91 | 1 |
| D6 | 2.45 | 3 | D8 | 1.54 | 1 |
| D2 | 2.38 | 2 | D9 | 1.43 | 0 |
| D7 | 2.32 | 2 | D10 | 0.98 | 0 |
| RefTB1 | 2.3 | 2 | Negative | 0 | 0 |

### Assay 10: Shift From M1 to M2 Microglia Phenotype

Assay 8 and 9 are measuring the fraction of microglia cells losing their M1 phenotype and gaining an M2 phenotype respectively. This assay combines loss of markers for M1 phenotype and gaining of M2 phenotype markers to reflect a shift from M1 to M2. This is combined in the same assay and give synergistic value, for example CD200R increase CD183 reduction. In this example the same conditions are used as in assay 8 and 9 but with an HMC3 to MSC ratio of and for both CD200R and CD183.Result: The shift from M1 to M2 is calculated as:

$$S h i f t s c o r e = \frac{C D 200 R f o l d i n c r e a s e}{1 - C D 183 s u p p r e s s i o n}$$

The donors with the highest shift score get the highest ranking score. The ranking score is later used in the final selection of donors.

TABLE 17

Illustrative example of shift score based on CD200R fold increase and CD183 suppression

| Donor | CD183^{∗} | CD200R | Shift score | Ranking score |
|---|---|---|---|---|
| D5 | 76% | 3 | 12.5 | 3 |
| D6 | 74% | 2.96 | 11.4 | 3 |
| D7 | 71% | 2.45 | 8.4 | 3 |
| D1 | 69% | 2.38 | 7.7 | 2 |
| D2 | 58% | 2.32 | 5.5 | 2 |
| RefTB1 | 56% | 2.3 | 5.2 | 2 |
| D8 | 54% | 2.08 | 4.5 | 2 |
| D3 | 49% | 1.91 | 3.7 | 1 |
| D4 | 39% | 1.54 | 2.5 | 1 |
| D9 | 32% | 1.43 | 2.1 | 0 |
| D10 | 22% | 0.98 | 1.3 | 0 |
| Negative | 0% | 0 | 0 | 0 |

^{∗} Note: The CD183 suppression is analysed for co-culture at a 1:1 ratio of HMC3 and MSC in this assay.

Alternative markers are for phenotype shift from M1 to M2:

M1 markers which decrease: B7-2/CD86, Integrin alpha V beta 3, MFG-E8, NO, ROS, RNS, CCL2/MCP-1, CCL3/MIP-1 alpha, CCL4/MIP-1 beta, CCL5/RANTES, CCL8/MCP-2, CCL11/Eotaxin, CCL12/MCP-5, CCL15/MIP-1 delta, CCL19/MIP-3 beta, CCL20/MIP-3 alpha, CXCL1/GRO alpha/KC/CINC-1, CXCL9/MIG, CXCL10/IP-10, CXCL11/I-TAC, CXCL13/BLC/BCA-1, CX3CL1/Fractalkin, MMP-3, MMP-9, Glutamate, IL-1 beta/IL-1F2, IL-2, IL-6, IL-12, IL-15, IL-17/IL-17A, IL-18/IL-1F4, IL-23, IFN_{Y}, TNF-alpha, Fc gamma RIII/CD16, Fc gamma RII/CD32, CD36/SR-B3, CD40, CD68/SR-D1, B7-1/CD80, MHC II, iNOS, COX-2.

M2 markers which increase: IL-1Ra/IL-1F3, IL-4, IL-10, IL-13, TGF-beta, CCL13/MCP-4, CCL14, CCL17/TARC, CCL18/PARC, CCL22/MDC, CCL23/MPIF-1, CCL24/Eotaxin-2/MPIF-2, CCL26/Eotaxin-3, FIZZ1/RELM alpha, YM1/Chitinase 3-like

3, CLEC10A/CD301, MMR/CD206, SR-Al/MSR, CD163, Arginase 1/ARG1, Transglutaminase 2/TGM2, PPAR gamma/NR1C3.

### Assay 11: Dendritic Cells

Fms-related tyrosine kinase 3-ligand (FLT3L) is a key regulator of DC commitment in hematopoiesis, which regulates the proliferation, differentiation and apoptosis of hematopoietic cells through the binding to FLT3. MSCs express FLT3L that binds to FLT3 on CD1 c+DCs to promote the proliferation and inhibit the apoptosis of tolerogenic CD1c+DCs. MSC expression of FLT3L measured by ELISA in co-culture with PBMC according to Assay 2, with or without stimulation with e.g. PHA or LPS. The fraction of cells being CD1c+ will increase as the Drug Substance Intermediate and/or Drug Substance, i.e. MSCs as described herein, induce tolerance which can be analyzed flow cytometry.

Co-culturing MSC with PBMC: MSC (2× 10⁵ cells/well) in 500 µl of working medium (RPMl1640 (ThermoFisher Scientific, cat no. 12633012) + 2 mM Glutamax (ThermoFisher Scientific, cat no. 35050061) + 100 U/ml Pest (ThermoFisher Scientific, cat no. 15140122) + 10% FBS (ThermoFisher Scientific, cat no. 16140071) are seeded in 12-well cell culture plates. The plates are incubated at 37° C., 5% CO₂ for 2 hours for plastic adherence of the cells. Lymphoprep™ kit is used for isolation of mononuclear cells from donated peripheral blood, retrieved from the blood central, according to manufacturer’s instructions (Stem Cell Technologies, cat no. 07801). PBMC (1×10⁶ cells /well) +PHA or LPS added as mitogen, are seeded in the 12-well cell culture plate and the co-culture is incubated for 72 h at 37° C., 5% CO₂

Analysis: The supernatant is labeled with anti-FLT3L antibody (MyBioSource, Inc. cat no MBS2035709) according to manufacturer’s instruction for ELISA and presence of soluble FLT3L is quantified with Spectramax microplate reader (Molecular Devices, Spectramax 190).

The CD1c+ fraction of dendritic cells is defined as CD11c+ and CD1c+ of the PBMCs, analyzed by flow cytometry (Becton, Dickinson and Company, Accuri C6 plus). The cells in suspension are labeled with anti-CD11c antibody and anti-CD1c antibody (ThermoFisher Scientific cat no 12-0116-42 and 12-0015-42 respectively) according to manufacturer’s instruction.

### Results

The Drug Substance Intermediates and/or Drug Substances are analyzed for FLT3L expression and receive a score based on the relative expression compared to the other samples analyzed. The fraction of CD1c+ cells of the CD11c+ cells from the supernatant after co-culture with Drug Substance Intermediates and/or Drug Substances are analyzed and receive a score based on the relative expression compared to the other samples analyzed.

TABLE 18

Illustrative example of relative FLT3L expression, CD1c+ positive cell fraction and the dendritic cell related score. The DC score is an average of the FLT3 and CD1c+ score

| Donor | FLT3L | CD1c+ | FLT3L score | CD1c+ score | DC score |
|---|---|---|---|---|---|
| D2 | 2.1 | 15% | 3 | 3 | 3 |
| D1 | 2.0 | 13% | 3 | 2 | 2.5 |
| D5 | 1.8 | 14% | 3 | 3 | 3 |
| D3 | 1.7 | 12% | 2 | 2 | 2 |
| D8 | 1.1 | 10% | 2 | 1 | 1.5 |
| RefTB1 | 1 | 12% | 2 | 2 | 2 |
| D7 | 0.9 | 15% | 2 | 3 | 2.5 |
| D6 | 0.9 | 13% | 1 | 2 | 1.5 |
| D10 | 0.8 | 8% | 1 | 0 | 0.5 |
| D9 | 0.4 | 7% | 0 | 0 | 0 |
| D4 | 0.2 | 9% | 0 | 1 | 0.5 |
| Negative | 0 | 0 | 0 | 0 | 0 |

The score later used in the final selection of donors can be FLT3L and/or CD1c+ or the combined score presented as DC score.

### Assay 11: FLT3L

Fms-related tyrosine kinase 3-ligand (FLT3L) is a key regulator of DC commitment in hematopoiesis, which regulates the proliferation, differentiation and apoptosis of hematopoietic cells through the binding to FLT3. MSCs express FLT3L that binds to FLT3 on CD1c+DCs to promote the proliferation and inhibit the apoptosis of tolerogenic CD1c+DCs. MSC expression of FLT3L measured by ELISA in co-culture with PBMC according to Assay 2, with or without stimulation with e.g. PHA or LPS.

Co-culturing MSC with PBMC: MSC (2× 10⁵ cells/well) in 500 µl of working medium (RPMl1640 (ThermoFisher Scientific, cat no. 12633012) + 2 mM Glutamax (ThermoFisher Scientific, cat no. 35050061) + 100 U/ml Pest (ThermoFisher Scientific, cat no. 15140122) + 10% FBS (ThermoFisher Scientific, cat no. 16140071) are seeded in 12-well cell culture plates. The plates are incubated at 37° C., 5% CO₂ for 2 hours for plastic adherence of the cells. Lymphoprep™ kit is used for isolation of mononuclear cells from donated peripheral blood, retrieved from the blood central, according to manufacturer’s instructions (Stem Cell Technologies, cat no. 07801). PBMC (1×10⁶ cells /well) +PHA or LPS added as mitogen, are seeded in the 12-well cell culture plate and the co-culture is incubated for 72 h at 37° C., 5% CO₂

Analysis: The supernatant is labeled with anti-FLT3L antibody (MyBioSource, Inc. cat no MBS2035709) according to manufacturer’s instruction for ELISA and presence of soluble FLT3L is quantified with Spectramax microplate reader (Molecular Devices, Spectramax 190).

### Results

The Drug Substance Intermediates and/or Drug Substances are analyzed for FLT3L expression and receive a score based on the relative expression compared to the other samples analyzed. The score is later used in the final selection of donors.

TABLE 18

Illustrative example of relative FLT3L expression.

| Donor | FLT3L | FLT3L score | Donor | FLT3L | FLT3L score |
|---|---|---|---|---|---|
| D2 | 2.1 | 3 | D7 | 0.9 | 2 |
| D1 | 2.0 | 3 | D6 | 0.9 | 1 |
| D5 | 1.8 | 3 | D10 | 0.8 | 1 |
| D3 | 1.7 | 2 | D9 | 0.4 | 0 |
| D8 | 1.1 | 2 | D4 | 0.2 | 0 |
| RefTB1 | 1 | 2 | Negative | 0 | 0 |

### Assay 12: CD1c

MSC immunosuppressive effect on dendritic cells can be analyzed as a phenotypic shift towards CD1c positive cells as MSCs promote the proliferation and inhibit the apoptosis of tolerogenic CD1c+DCs.

The fraction of cells being CD1c+ will increase as the Drug Substance Intermediate and/or Drug Substance, i.e. umbilical cord derived MSCs, induce tolerance which can be analyzed flow cytometry.

Co-culturing MSC with PBMC: MSC (2× 10⁵ cells/well) in 500 µl of working medium (RPMl1640 (ThermoFisher Scientific, cat no. 12633012) + 2 mM Glutamax (ThermoFisher Scientific, cat no. 35050061) + 100 U/ml Pest (ThermoFisher Scientific, cat no. 15140122) + 10% FBS (ThermoFisher Scientific, cat no. 16140071) are seeded in 12-well cell culture plates. The plates are incubated at 37° C., 5% CO₂ for 2 hours for plastic adherence of the cells. Lymphoprep™ kit is used for isolation of mononuclear cells from donated peripheral blood, retrieved from the blood central, according to manufacturer’s instructions (Stem Cell Technologies, cat no. 07801). PBMC (1×10⁶ cells /well) +PHA or LPS added as mitogen, are seeded in the 12-well cell culture plate and the co-culture is incubated for 48 h at 37° C., 5% CO₂

### Analysis

The CD1 c+ fraction of dendritic cells is defined as CD11c+ and CD1 c+ of the PBMCs, analyzed by flow cytometry (Becton, Dickinson and Company, Accuri C6 plus). The cells in suspension are labeled with anti-CD11c antibody and anti-CD1c antibody (ThermoFisher Scientific cat no 12-0116-42 and 12-0015-42 respectively) according to manufacturer’s instruction.

### Results

The effect of Drug Substance Intermediates and/or Drug Substances on dendritic cells is quantified as the fraction of CD1c+ cells of the CD11c+ cells from the supernatant after co-culture. Dendritic cells cultured with Drug Substance Intermediates and/or Drug Substances are analyzed and receive a score based on the relative induction of CD1c+ expression compared to the other samples analyzed. The score later used in the final selection of donors can be FLT3L and/or CD1c+ or the combined score presented as DC score.

TABLE 18

CD1c+ positive cell fraction of the dendritic cells related score.

| Donor | CD1c+ | CD1c+ score | Donor | CD1c+ | CD1c+ score |
|---|---|---|---|---|---|
| D2 | 15% | 3 | D7 | 15% | 3 |
| D1 | 13% | 2 | D6 | 13% | 2 |
| D5 | 14% | 3 | D10 | 8% | 0 |
| D3 | 12% | 2 | D9 | 7% | 0 |
| D8 | 10% | 1 | D4 | 9% | 1 |
| RefTB1 | 12% | 2 | Negative | 0 | 0 |

### Assay 13: Dendritic Cells

The combined result of Fms-related tyrosine kinase 3-ligand (FLT3L) expression of the Drug Substance Intermediate and/or Drug Substance (assay 13) and the fraction of CD1 c+ dendritic cells after coculture with the Drug Substance Intermediate and/or Drug Substance (assay 12) is combined to give a dendritic cell score.

### Results

The Drug Substance Intermediates and/or Drug Substances are analyzed for FLT3L expression and receive a score based on the relative expression compared to the other samples analyzed. The fraction of CD1 c+ cells of the CD11c+ cells from the supernatant after co-culture with Drug Substance Intermediates and/or Drug Substances are analyzed and receive a score based on the relative expression compared to the other samples analyzed.

TABLE 18

Illustrative example of relative FLT3L expression, CD1c+ positive cell fraction and the dendritic cell related score. The DC score is an average of the FLT3 and CD1c+ score

| Donor | FLT3L | CD1c+ | FLT3L score | CD1c+ score | DC score |
|---|---|---|---|---|---|
| D2 | 2.1 | 15% | 3 | 3 | 3 |
| D1 | 2.0 | 13% | 3 | 2 | 2.5 |
| D5 | 1.8 | 14% | 3 | 3 | 3 |
| D3 | 1.7 | 12% | 2 | 2 | 2 |
| D8 | 1.1 | 10% | 2 | 1 | 1.5 |
| RefTB1 | 1 | 12% | 2 | 2 | 2 |
| D7 | 0.9 | 15% | 2 | 3 | 2.5 |
| D6 | 0.9 | 13% | 1 | 2 | 1.5 |
| D10 | 0.8 | 8% | 1 | 0 | 0.5 |
| D9 | 0.4 | 7% | 0 | 0 | 0 |
| D4 | 0.2 | 9% | 0 | 1 | 0.5 |
| Negative | 0 | 0 | 0 | 0 | 0 |

The score later used in the final selection of donors can be FLT3L and/or CD1 c+ or the combined score presented as DC score.

### Assay 14: Regulatory T-Cells

T regulatory cells are identified as a subpopulation of the CD4+CD25+ T cell population with the capacity to suppress an immune response. This subpopulation may be further characterized by lack of expression of CD127 or positive expression of FoxP3. This fraction of cells will increase when exposed to the Drug Substance Intermediate and/or Drug Substance, i.e. umbilical cord derived MSCs, which can be analyzed by flow cytometry.

Co-culturing of MSC with PBMC: MSC (2 × 10⁵ cells/well) in 500 µl of working medium (RPMI1640 (ThermoFisher Scientific, cat no. 12633012) + 2 mM Glutamax (ThermoFisher Scientific, cat no. 35050061) + 100 U/ml Pest (ThermoFisher Scientific, cat no. 15140122) + 10% FBS (ThermoFisher Scientific, cat no. 16140071) are seeded in 12-well cell culture plates. The plates are incubated at 37° C., 5% CO₂ for 2 hours for plastic adherence of cells. Lymphoprep™ kit is used for isolation of mononuclear cells from donated peripheral blood, retrieved from the blood central, according to manufacturer’s instructions (Stem Cell Technologies, cat no. 07801). PBMC (1× 10⁶ cells/well=500 µl) are suspended in working medium and added to the 12-well plates and co-culture is continued for 24 hours at 37° C., 5% CO₂.

### Analysis

The cells in suspension are labeled with CD4 antibody and CD25 antibody (ThermoFisher Scientific cat no 15-0041-82 and 48-0259-42, respectively) according to manufacturer’s instruction. The cells may be further characterized by lack of expression of CD127 or positive expression of FoxP3.

### Results

The fraction of CD25 positive (optionally CD127 negative or FoxP3 positive) CD4+ cells from the supernatant after co-culture with Drug Substance Intermediates and/or Drug Substances are analyzed and receive a relative score based on the fraction of CD25+ cells compared to the other samples analyzed. The score is later used in the final selection of donors.

TABLE 19

Illustrative example of CD25+ positive cells.

| Donor | CD25+ percentage | CD25+ ranking score | Donor | CD25+ percentage | CD25+ ranking score |
|---|---|---|---|---|---|
| D7 | 8.1 | 3 | D2 | 4.0 | 2 |
| D6 | 6.0 | 3 | D1 | 3.4 | 1 |
| D5 | 5.8 | 3 | D8 | 2.7 | 1 |
| D4 | 4.7 | 2 | D9 | 1.8 | 0 |
| D3 | 4.4 | 2 | D10 | 1.5 | 0 |
| RefTB1 | 4.1 | 2 | Negative | 2.1 | 0 |

### Assay 15: Changing Monocyte Phenotype

Monocytes originate from myeloid precursors in the bone marrow and they can enter CNS during inflammation. Classically, a monocyte is CD14++ CD16-. These classical monocytes are highly plastic and upon recruitment to inflamed tissues, they can change to macrophages or dendritic cells. Non classical monocytes are CD14+ CD16++ and involved in tissue homeostasis and local regeneration. MSC can change the monocyte phenotype from classical to non-classical.

Co-culturing MSC with PBMC: MSC (5× 10⁴ cells/ tube) in 500 µl of working medium (RPMl1640 (ThermoFisher Scientific, cat no. 12633012) + 2 mM Glutamax (ThermoFisher Scientific, cat no. 35050061) + 100 U/ml Pest (ThermoFisher Scientific, cat no. 15140122) + 10% FBS (ThermoFisher Scientific, cat no. 16140071) are seeded in polypropylene culture tubes. Lymphoprep™ kit is used for isolation of mononuclear cells from donated peripheral blood, retrieved from the blood central, according to manufacturer’s instructions (Stem Cell Technologies, cat no. 07801). Monocytes from mononuclear cells, positively selected with magnetic beads coupled to monoclonal anti human CD14 antibodies from Miltenyi Biotec, (Germany # 130-050-201) are isolated according to manufacturer’s instructions. 2×10⁵ monocytes in 500 µl of working medium, is seeded into the MSC-containing polypropylene tubes. The co-culture is incubated for 24 hours at 37° C., 5% CO₂.

### Analysis

Cells are collected and washed twice with DPBS+2%FBS+2 µM EDTA and labelled with Anti CD14 PE (Thermofisher, Catalog # 12-0149-42) and anti-CD16 FITC (Thermofisher Catalog # 11-0168-42). The increasing expression of CD16 and the decreasing percentage of CD14++ CD16- in monocytes in co-culture with and without Drug Substance Intermediates and/or Drug Substances are compared. The donor which have highest fold induction of CD16 expression and highest suppression of CD14++CD16- will get the highest score at final donor selection.

TABLE 20

Illustrative example of CD16 positive cells and suppression percentage of CD14++CD16-

| Donor | CD16++ fold induction | Ranking score CD16++ fold induction | Donor | CD14++CD16-Suppression percentage | Ranking score CD14++ suppression percentage | Monocyte ranking score |
|---|---|---|---|---|---|---|
| D7 | 20 | 3 | D7 | 28 | 2 | 2.5 |
| D6 | 15 | 3 | D6 | 33 | 3 | 3 |
| D5 | 15 | 3 | D5 | 22 | 2 | 2.5 |
| D4 | 12 | 2 | D4 | 18 | 2 | 2 |
| D3 | 12 | 2 | D3 | 36 | 3 | 2.5 |
| RefTB1 | 10 | 2 | RefTB1 | 21 | 2 | 2 |
| D2 | 10 | 2 | D2 | 14 | 1 | 1.5 |
| D1 | 8 | 1 | D1 | 20 | 1 | 1 |
| D8 | 8 | 1 | D8 | 31 | 3 | 2 |
| D9 | 6 | 0 | D9 | 13 | 0 | 0 |
| D10 | 6 | 0 | D10 | 11 | 0 | 0 |
| Negative | 5 | 0 | Negative | 5 | 0 | 0 |

The score later used in the final selection of donors can be CD16++ and/or CD14++ suppression percentage or the combined score presented as monocyte ranking score.

### Assay 16: Expression of ACE2 Receptor and TMPRSS2 mRNA in WJMSCs and Pro Trans

ACE2 receptor is the entry point on the surface of target cells for infection by coronavirus spike proteins. Co-expression of the serine protease TMPRSS2 by the target cells allows for coronavirus spike protein priming.

Evaluating mRNA expression of ACE2 receptor and TMPRSS2: Total RNA was isolated from the control WJMSCs and ProTrans product (CB1, CB2 and TB1) using an RNeasy mini kit (QIAGEN). cDNA was generated from each sample using the high capacity cDNA reverse transcriptase kit. RT- samples were generated as controls for each experimental sample. Quantitative PCR (QPCR) was run using Fast SYBR Green Master Mix (Applied Biosystems) with primers designed to target the human ACE2 receptor (F 5′TTCTGTCACCCGATTTTCAA 3′ (SEQ ID NO:1; R 5′TCCCAACAATCGTGAGTGC 3′ (SEQ ID NO:2)), human TMPRSS2 (F 5′CGCTGGCCTACTCTGGAA 3′ (SEQ ID NO:3); R 5′CTGAGGAGTCGCACTCTATCC 3′ (SEQ ID NO:4)) and human RPL13A (housekeeping gene; F 5′CCTGGAGGAGAAGAGGAAAGAGA 3′ (SEQ ID NO:5); R 5′TTGAGGACCTCTGTGTATTTGTCAA 3′ (SEQ ID NO:6)). Cycling was performed as per the manufacturer’s instructions for Fast Sybr Green amplification. A total of 40 cycles were performed.

Analysis: Average Cq values from a technical repeat of three runs were compared with the average Cq value of the equivalent reverse transcriptase (RT) negative control. Where values were higher than the respective RT- sample, the gene was deemed to not be expressed. Where values were lower than the respective RT- samples, the gene was deemed to be expressed. All samples tested were negative for ACE2 receptor and TMPRSS2. All samples were positive for the house-keeping gene RPL 13A.

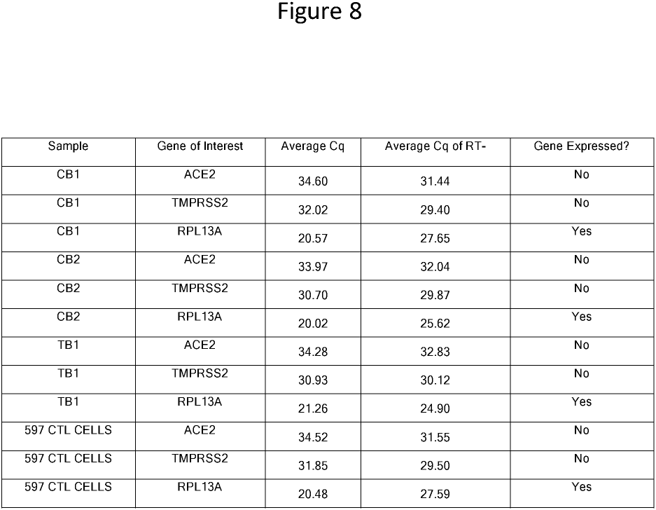

Results:FIG. 8 shows the results from analysis for mRNA expression of the ACE2 receptor, TMPRSS2 and house-keeping gene RPL13A in control WJ-MSC cells and ProTrans product (CB1, CB2 and TB1).

### Example 4

The present Example describes the process of selection of the MSC populations derived from the donors based on the characteristics described in Example 4 resulting in a subset of cells populations for pooling to obtain the inventive pooled allogeneic composition.

### Material and Methods

Analysis and ranking: The Drug Substance Intermediate samples are analyzed with the assays described above (IDO, proliferation, PGE2, HLA-G, Morphology and Fluorospot). Ranking of samples is performed as described below:

1. The IDO assay described above is conducted two times with duplicate cell culture samples and each sample is analyzed in triplicates with ELISA. Earlier batches pooled allogeneic MSCs are used as reference samples. The IDO assay contains control samples, analyzed with each run and results generated from the analysis of control samples are evaluated for acceptability using appropriate statistical methods.

Acceptable range of the two controls are according to manufacturer’s specification. An example of acceptable ranges is: Kynurenine (µmol/L) control 1: 0.53 -1,33 and control 2: 1.78-4.15; Tryptophan (µmol/L) control 1: 15.0-35.0 and control 2: 31.2-72.8. Quality criteria employed for assay are: IDO controls are within the specified range and IDO activity (reference sample) > 60-fold, i.e. the fold induction of IDO activity between interferon gamma (IFN_{Y}) reference sample compared to unstimulated reference sample.

The Drug Substance Intermediates are ranked based on their relative IDO expression.

2. The Proliferation assay described above is conducted two times with duplicate cell culture samples and each sample is analyzed in triplicates with FACS. The samples impact on PBMC proliferation is presented as proliferation index, PI. Earlier batches pooled allogeneic MSCs are used as reference samples and PBMC stimulated with PHA in absence of MSC is used as positive control. Quality criteria employed for assay are: Proliferation Index (positive control) > 1.5 and Proliferation Index (reference) 0.9-1.3.

The Drug Substance Intermediates are ranked based on their relative Proliferation Index

3. The PGE2 assay is conducted two times with duplicate cell culture samples and each sample is analyzed in triplicates with ELISA. The kit includes standards for establishing a standard curve for each experiment. Earlier batches pooled allogeneic MSCs are used as reference samples and the samples are compared based on the level of PGE2 expression in presence of PBMC activated by PHA. Quality criteria employed for assay are: PGE2 expression (reference) 5-15 ng/ml and Standard curves R2 > 0.95.

The Drug Substance Intermediates are ranked based on their relative PGE2 expression.

4. The HLA-G assay is conducted two times with duplicate cell culture samples and each sample is analyzed in triplicates with ELISA or FACS. Earlier batches pooled allogeneic MSCs are used as reference samples and the samples are compared based on the level of HLA-G expression in presence of PBMC activated by PHA. The ELISA kit includes standards for establishing a standard curve for each experiment. Quality criteria employed for assay are: Soluble HLA-G expression (reference) >3 U/ml, Standard curves R2 > 0.95 and Intracellular HLA-G expression (reference) >5%, The Drug Substance Intermediates are ranked based on their relative intracellular and/or soluble expression of HLA-G.

5. The morphology assessment is conducted by laboratory personnel with long experience in MSC culturing. Earlier batches pooled allogeneic MSCs are used as reference samples and the samples assessed based on: size of cell (normal or big); size of nuclei (normal or big); shape of cell (normal or abnormal); and ration between cell and nuclei size (normal or abnormal). Quality criteria employed for assay are:

>90% normal cells according to all four criteria. Reference sample has > 90% normal cells. The Drug Substance Intermediates that have more than 10% abnormal cells are disqualified. The Drug Substance Intermediates are ranked based on the frequency of abnormal cells.

6. The Fluorospot assay is conducted two times with triplicate cell culture samples. Earlier batches pooled allogeneic MSCs are used as reference samples. The Drug Substance Intermediates are ranked based on their relative expression or suppression of specific proteins.

7. Microglia proliferation assay is conducted two times with at least duplicate cell culture samples. Earlier batches pooled allogeneic MSCs are used as reference samples and microglia proliferation in presence of mitogen and absence of MSC is used as negative control. The Drug Substance Intermediates are ranked based on their relative ability to suppress microglia proliferation as measured by growth index, proliferation index or proliferation percentage.

8.-10. Microglia expression assays are conducted two times with at least duplicate cell culture samples. Earlier batches pooled allogeneic MSCs are used as reference samples. Mitogen stimulated microglia, cultured without MSC is used as negative control. The Drug Substance Intermediates are ranked based on their relative increase of M2 markers and/or decrease in expression of M1 markers and/or a combinatorial shift from M1 to M2 phenotype.

11. Dendritic cell assays is conducted two times with at least duplicate cell culture samples. Earlier batches pooled allogeneic MSCs are used as reference samples.

Outliers and disqualification of samples: ELISA and FACS are analyzed in triplicates from each cell culture well. Only one of the three triplicates can be regarded as an outlier. Measurements from a cell culture well are analyzed for outliers if the coefficient of variance (CV) is > 10%. The replicate which is deviating most from the average is considered an outlier if the exclusion of the replicate will decrease CV with > 3% when removed from the analysis. Such outliers are excluded from the analysis without further justifications.

The analysis of a single cell culture well is disqualified if the CV > 20% after outlier analysis has been conducted. Three or more disqualified cell culture wells in the same experiment will disqualify the experiment.

Overall assessment: The selection of Drug Substance Intermediates is an overall assessment of the assays according to a point system presented in Table 21 below. Each assay generates a ranking score and in this final selection, the ranking score of all the assays is summarized by addition.

Selecting 5 donors from the 10 donors can be accomplished by conducting at least 2 of IDO, PGE2 and Proliferation assay with at least 1 of the assays microglia proliferation, microglia M1 suppression, microglia M2 fold increase, Dendritic cell tolerogenicity or Regulatory T cell described in Example 3. Illustrative minimal selection algorithms with added value selection is presented in Table 21 and 22. Here ranking values are added for each assay to obtain an additive total score.

TABLE 21

Example of selection based on additive total score

| Donor (DX) | IDO | PI | Microglia Growth Index | Total score | Selected |
|---|---|---|---|---|---|
| D1 | 2 | 2 | 2 | 6 | yes |
| D2 | 1 | 1 | 0 | 2 | no |
| D3 | 2 | 2 | 1 | 5 | no |
| D4 | 3 | 2 | 2 | 7 | yes |
| D5 | 3 | 3 | 3 | 9 | yes |
| D6 | 2 | 3 | 3 | 8 | yes |
| D7 | 3 | 3 | 3 | 9 | yes |
| D8 | 1 | 1 | 2 | 4 | no |
| D9 | 0 | 0 | 0 | 0 | no |
| D10 | 0 | 0 | 0 | 0 | no |

TABLE 22

Example of selection based on additive total score

| Donor (DX) | IDO | PGE2 | Monocyte assay | Total score | Selected |
|---|---|---|---|---|---|
| D1 | 2 | 2 | 1 | 5 | no |
| D2 | 1 | 1 | 1.5 | 3.5 | no |
| D3 | 2 | 2 | 2.5 | 6.5 | yes |
| D4 | 3 | 3 | 2 | 8 | yes |
| D5 | 3 | 3 | 2.5 | 8.5 | yes |
| D6 | 2 | 2 | 3 | 7 | yes |
| D7 | 3 | 3 | 2.5 | 8.5 | yes |
| D8 | 1 | 1 | 2 | 4 | no |
| D9 | 0 | 0 | 0 | 0 | no |
| D10 | 0 | 0 | 0 | 0 | no |

Alternatively selecting 5 of the 10 donors is done by assigning a weight the assays, thus allowing an analysis to influence the selection of more or less donors. An example would be to put a factor two on microglia assay in Table 21 and decrease the importance of proliferation of peripheral blood lymphocytes to half. Weighed ranking scores are added to obtain a weighed total score. The results from Table 21 based on weighed total score are shown in Table 23:

TABLE 23

Example of selection based on additive total score

| Donor (DX) | IDO | PI (x0.5) | Microglia GI (x2) | Total score | Selected |
|---|---|---|---|---|---|
| D1 | 2 | 1 | 4 | 7 | yes |
| D2 | 1 | 0.5 | 0 | 1.5 | no |
| D3 | 2 | 1 | 2 | 5 | no |
| D4 | 3 | 1 | 4 | 8 | yes |
| D5 | 3 | 1.5 | 6 | 10.5 | yes |
| D6 | 2 | 1.5 | 6 | 9.5 | yes |
| D7 | 3 | 1.5 | 6 | 10.5 | yes |
| D8 | 1 | 0.5 | 4 | 5.5 | no |
| D9 | 0 | 0 | 0 | 0 | no |
| D10 | 0 | 0 | 0 | 0 | no |

Alternatively selecting 5 of the 10 donors is done by assigning a weight the assays, thus allowing an analysis to influence the selection of more or less donors. An example would be to put a factor three on IDO assay in Table 15 and increase the importance of the monocyte assay by factor 2. Weighed ranking scores are added to obtain a weighed total score. The results from Table 22 based on weighed total score are shown in Table 24:

TABLE 24

Example of selection based on additive total score of 3 assays with different weight

| Donor (DX) | IDO (x3) | PG E2 | Monocyte assay (x2) | Total score | Selected 5 |
|---|---|---|---|---|---|
| D1 | 6 | 2 | 2 | 10 | no |
| D2 | 5 | 1 | 3 | 9 | no |
| D3 | 6 | 2 | 5 | 13 | yes |
| D4 | 9 | 3 | 4 | 16 | yes |
| D5 | 9 | 3 | 5 | 17 | yes |
| D6 | 6 | 2 | 6 | 14 | yes |
| D7 | 9 | 3 | 5 | 17 | yes |
| D8 | 3 | 1 | 4 | 8 | no |
| D9 | 0 | 0 | 0 | 0 | no |
| D10 | 0 | 0 | 0 | 0 | no |

An example of a selection algorithm based on 11 assays is presented in Table 25.

TABLE 25

Example of selection based on additive total score of 11 assays with the same weight

| | | | | | | | | | | | | | |
|---|---|---|---|---|---|---|---|---|---|---|---|---|---|
| Assay | 1 | 2 | 3 | 4 | 5 | 6 | 7 | 10 | 13 | 14 | 15 | Total Score | Selecte d |
| D1 | 2 | 2 | 2 | 2 | 2 | 2 | 2 | 2 | 3 | 1 | 1 | 21 | Yes |
| D2 | 1 | 1 | 1 | 1 | 2 | 2 | 0 | 2 | 2, 5 | 2 | 1,5 | 16 | No |
| D3 | 2 | 2 | 2 | 2 | 1 | 1 | 1 | 1 | 2 | 2 | 2,5 | 18,5 | No |
| D4 | 3 | 2 | 3 | 3 | 2 | 2 | 2 | 1 | 0, 5 | 2 | 2 | 22,5 | Yes |
| D5 | 3 | 3 | 3 | 3 | 3 | 3 | 3 | 3 | 3 | 3 | 2,5 | 32,5 | Yes |
| D6 | 2 | 3 | 2 | 2 | 3 | 3 | 3 | 3 | 1, 5 | 3 | 3 | 28,5 | Yes |
| D7 | 3 | 3 | 3 | 3 | 3 | 3 | 3 | 3 | 2, 5 | 3 | 2,5 | 32 | Yes |
| D8 | 1 | 1 | 1 | 1 | 1 | 1 | 2 | 2 | 1, 5 | 1 | 2 | 14,5 | No |
| D9 | 0 | 0 | 0 | 0 | 0 | 0 | 0 | 0 | 0 | 0 | 0 | 0 | No |
| D10 | 0 | 0 | 0 | 0 | 0 | 0 | 1 | 0 | 0, 5 | 0 | 0 | 1,5 | No |

### Results

The 5 Drug Substance Intermediates (DX) with the highest total score (additive/simple or weighed) are selected for manufacturing of the isolated pooled allogeneic MSC population, i.e. the Drug Product, as disclosed herein. Thus, the isolated, pooled allogeneic population comprises MSCs derived from 5 different donors, which MSCs fulfil the functional, morphological and safety criteria as disclosed herein.

### Example 5

The present Example describes the process of manufacturing the Final Product, which is a single cell suspension comprising excipients as described below. Said Final Product is filled in transfer bags suitable for cryopreservation and frozen according to predefined temperature curves as described below.

TABLE 26

Composition packaging of Final Product″ Alburex 5 (CSL Behring), 50 G/L; human serum albumin 50 g/L; purity of protein > 96%, sodium N- acetyltryptophanate, sodium caprylate, sodium chloride
CELL SUSPENSION COMPONENTS

| Component | Function | Quantity | Quality |
|---|---|---|---|
| Allogeneic WJMSCs | Active Substance | 100×10⁶ cells | In-house |
| 5% Human Serum Albumin^{∗} | Component of Cryoprotectant | 90% vol/vol (4.5 mL) | Ph.Eur |
| Dimethyl Sulfoxide (DMSO)^{∗∗} | Component of Cryoprotectant | 10% vol/vol (0.5 mL) | Ph.Eur |
| PACKAGING COMPONENTS | | | |
| Component | Description | Function | Quality |
| Cryobags | Bag; ethylene vinyl acetate | Primary container | Ph.Eur |
| Cassette | Container; aluminum alloy | Protective container | In-house |
| Label | Cryo label | Information | In-house |

^{∗∗} WAK Chemie, Cat.no. WAK-DMSO-50.

### Materials and Methods

### Pooling of Donors

Only donor samples that pass all acceptance criteria are considered for pooling. The Drug Substances used are selected according to Example 4 and 5. The pooling of Drug Substance in passage 2 or 3 is directly followed by cryopreservation. The Drug Product is thus obtained. Importantly, the Drug Product is not subjected to any further culturing or expansion.

### Formulation and Packaging of Drug Product

The Final Product is a 5 mL of cell suspension and is presented in cryobags. The composition of cryopreserved Final Product, comprising the Drug product, is shown in Table 26.

### Results

Thus, a resulting Final Product is obtained as disclosed herein.

### Example 6

The present Example describes evaluation of the stability of the Final Product after cryopreservation. It shows that the Final Product is stable for at least 2 hours post thawing.

### Materials and Methods

The inventive composition is shipped on liquid nitrogen or on dry ice in cryo bags containing 5 ml of cell suspension with 30, 50, 60 or 100 million cells per bag (the Final Product). The cryo bag is thawed in water bath (37° C.) and directly diluted with autologous spinal fluid or lactated Ringer’s solution, usually 10 ml. The injection solution of 15 ml is then ready for infusion. The viability of the cells is analyzed by taking a sample from the infusion bag at different time points.

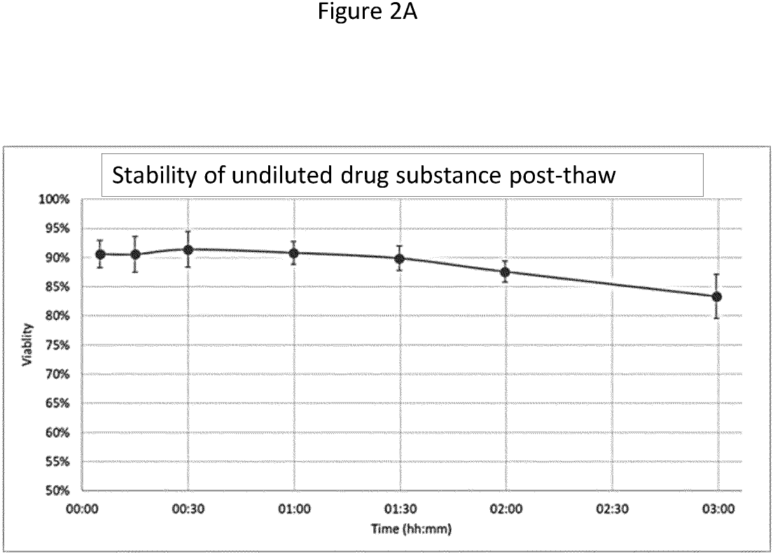

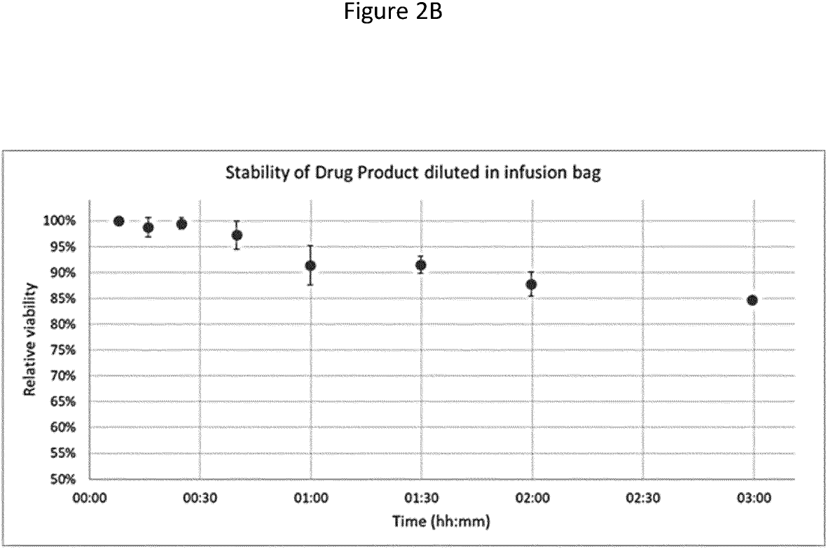

Stability of the Drug product is investigated by flow cytometry analysis to the apoptotic marker 7AAD. The Drug Product is stable for more than 2 hours post thaw undiluted (FIG. 2a). The Drug Product is diluted in sodium chloride infusion solution by transferring the drug product from the cryo bag to a saline infusion bag (Baxter). Aliquots are taken at different time points while kept at room temperature (FIG. 2b).

### Results

Viability: The Drug Product is regarded stable until the time point when the viability has decreased to 80 % of the viability measured instantly after thawing. The Drug Product has been tested for MSC specific cell surface markers and culturing potency at the stability time limit of 2 hours. The Drug Product has shown sustained characteristics after 2 hours and acceptable viability for both diluted and undiluted.

Conclusion: The analyzed batch the inventive composition fulfills quality criteria with cell viability.

### Example 7

The present Example provides a summary of the clinical study design of intravenous administration of the inventive pooled allogeneic MSC composition into patients diagnosed with COVID-19. Safety and tolerance of intravenous infusion of inventive pooled allogeneic MSC composition in adult patients diagnosed with COVID-19 over 24 months. Any adverse events will be reported and potential causal relationship with the Final product will be investigated.

Study Objectives: The primary objective of the study is to investigate the safety and tolerance of intravenous infusion of the inventive pooled allogeneic MSC composition in adult patients with COVID-19. Secondary objectives include to assess the effect of the Final Product on patient clinical status including mortality to day 7, 15 and 30 as assessed on the 9-point ordinal scale (0. No clinical or virological evidence of infection. 1. No limitations of activities. 2. Ambulatory, limitation of activities. 3. Hospitalized, not requiring supplemental oxygen. 4. Hospitalized, requiring supplemental oxygen. 5. Hospitalized, on non-invasive ventilation or high flow oxygen devices. 6. Hospitalized, intubated and on mechanical ventilation. 7. Hospitalized, ventilation + additional organ support - pressors, RRT, ECMO, 8. Death); time to clinical improvement of one category from admission on the 9 point ordinal scale; effect of the Final Product on lung damage using imaging techniques; duration of hospitalisation and intensive care unit stay; kinetics of COVID-19 viral load after infusion with the Final Product; evolution of biological markers of liver, myocardium and inflammation after infusion with the Final Product; tolerance of the Final Product for severe COVID-19 respiratory conditions.

Study design: This is a an open, dose escalating Phase IB Clinical Trial.

First cohort of three patients will receive the dose of 25 Million MSCs per patients. In case of no toxicity among the first cohort, the second cohort of three patients will receive the dose of 100 Million MSCs per patients. If the first patient experiences one toxicity grade 3 or 4 related to the study drug, the DSMB of the study will provide the sponsor with a recommendation on how to proceed. The sponsor will then take a decision on stopping the trial. If no AE grade 3 or 4 is observed post dosing of the first patient the next patient will be dosed as planned. A total of 3 patients will be treated with a dose of 25 Million MSCs. If there are no severe AEs observed in the first cohort the trial will proceed and dosing of the second cohort of three patients at the dose of 100 Million MSCs per patients will be continued. Rules for the third cohort will be the same. In case of no toxicity grade 3 or 4 related to the study drug among the first and the second cohorts and individuals within each cohort, the third cohort of three patients will receive the dose of 200 Million MSCs per patients.

Screening and informed consent: After signature of the informed consent, the following assessments will be performed to determine eligibility requirements as specified in the inclusion and exclusion criteria, and will be scheduled before the inclusion of the patient in the study:
Demography, medical history (treatments, co-morbidities and allergies)
Confirm the positive SARS-CoV-2 test result by PCR in any specimen before inclusion
Classification of the pneumonia assessed by SpO₂ measurement and need of supplemental oxygen
Blood test for screening laboratory evaluations, if last results >72 h from day of inclusion:
   ✓ Haematology (WBC, haemoglobin, platelet count)
   ✓ Biochemistry (AST, ALT total bilirubin, serum creatinine, blood electrolytes (including kaliemia), CPK, NT-proBNP, CK-Mb, haptoglobin, Troponin I, procalcitonin, LDH, ferritinaemia, CRP)
   ✓ Hemostasis markers (D-dimer levels, INR, fibrinogen, anti-thrombin and antiXa activity)
   ✓ Serological profile of HIV, syphilis, HEP B, HEP C
Investigate tuberculosis infection or prior exposure
Urinary test for cytobacterium evaluation, if last results >72 h from day of inclusion
Urine or serum pregnancy test (in women of childbearing potential) Treatment - Intravenous administration of WJMSCs and acute monitoring: Post- screening and study inclusion study subjects will receive an intravenous injection of the WJMSCs.

The inventive pooled allogeneic MSC composition will be delivered to the study clinic in liquid nitrogen. The drug product is thawed in water bath for 3-5 minutes. The Final Product is diluted in 100 ml of saline prior to administration.

During the hospitalization the subject will be observed by research nurses for any Adverse Events during and immediately following the intravenous injection of the inventive pooled allogeneic MSC composition.

Follow-up: Patients will be evaluated post-treatment according to Table 27.

Final Follow-up Visit: All study subjects, will have a final follow-up visit 24 months after MSC composition treatment. At this visit patients will undergo the following procedures as illustrated in Table 27.

TABLE 27

Summary table of evaluation post treatment

| | | | | | | | | | | | | |
|---|---|---|---|---|---|---|---|---|---|---|---|---|
| | Eligibi -lity | inclusion | Day 0 Before MSC infusion (Baseline) | Day 0 3 hours after MSC infusion | 1 Day 1 post-MSC | Day 3 post-MSC | Day 5 post-MSC | Day 8² post-MSC | Day 15² post-MSC | Day of discharge OR Day 30² | Follow up: Months 2, 3, 4, 5, 6 (+/- 2 weeks) post- MSC | Long term follow up: Month 12, 18, 24 (+/-2 weeks) post-MSC |
| Informed Consent¹ | x | | | | | | | | | | | |
| Medical history (treatments and comorbidities) and demography | x | | | | | | | | | | | |
| Clinical status (7 point ordinal scale) | | | Everyday | | | | | | | | | |
| Serological status (HIV, syphilis, HEP B, HEP C) | x | | | | | | | | | | | |
| Urinary test (for bacterial infection) | x | | | | | | | | | | | |
| β-HCG (urine) | x | | | | | | | | | | | |
| SARS-CoV-2 RT-PCR in any specimen | x | | | | | | | | | | | |
| SARS-CoV-2 RT-PCR in throat swab | | | x | | | x | x | x | | x | | |
| O₂ Saturation (twice a day)³ | x | Everyday | | | | | | | | | | |
| O₂ requirement (twice a day)³ | x | | Everyday | | | | | | | | | |
| Haematology (WBC, haemoglobin, platelets count) | x | | x | | x | x | x | x | x | x | x | x |
| Biochemistry (AST, ALT total bilirubin, serum reatinine,Na+ K), CPK, NT-proBNP, CP-Mb, haptoglobin, Troponin 1, procalcitonin, LDH, ferritinaemia, CRP) | x | | x | | x | x | x | x | x | x | x | x |
| Hemostasis markers (D-dimer levels, INR, fibrinogen, anti-thrombin and antiXa activity ) | | | x | | x | x | x | x | x | x | x | x |
| Chest X ray, CT scan, or doppler ultrasound (optional⁴) | Prior to MSC infusi on | | After MSC infusion, prior to hospital discharge | | | | | | | | | |
| Adverse events | | | Everyday if applicable (or at each visit) | | | | | | | | | |
| Plasma Biobanking | | | x⁵ | x | x | | | x | x | x | | |

1 : Informed consent should be signed before any study procedure
2: All procedures need to be conducted according to the table up to D8. This protocol is designed to follow patients until Day 30. However, if the patient is discharged prior to D30, examinations, with the exception of adverse events, related to Day 30 follow- up visit will be performed on discharge. This means that patients will be followed-up to D15 OR the point of discharge (if discharge take place after D15). Adverse events will be followed up to Day 30 in the form of a telephone call once a week to the patient if they have been discharged from care prior to this timepoint.
3: worst values/requirements are kept among the 2 daily measurements
4: These examinations are not required by the trial but can be exploited if done before and after MSC infusion for clinical need.
5: Immediately prior to MSC infusion

End of trial: The end of study is defined as the last patient’s last follow-up. The Principle Investigator has the right to at any time terminate the study for clinical or administrative reasons. The study may be prematurely terminated due to a high number of serious adverse events related to the ATMP or if the enrolment process cannot be completed within a reasonable time frame.

Decision on premature study termination will be made by sponsor/principal investigator. Study termination will be reported to the MPA within 90 days, or within 15 days if the study is terminated prematurely. The Investigators will inform participants and ensure that the appropriate follow-up is arranged for all involved. A summary report of the study will be submitted to the Medical Products Agency, MPA, within one year after study termination. All patients will be followed-up for two years post study treatment. Patients will be followed-up regarding safety aspects assessed by a study investigator at all follow-up visits.

Alternatively, intrathecal delivery is used in the clinical study. The clinical study design is the same as for the study comprising intrathecal administration, with the following exception: the Final Product will be delivered in a volume of 10-15 ml..

Alternatively the intravenous infusion of the Final Product may constitute a Phase II randomized, double-blinded study using a 1:1 randomisation strategy for Final Product to Placebo as defined below:.

Study objectives: To evaluate the efficacy of a single infusion of a fixed dose of Final Product (100 Million MSCs per patient) in the treatment of “severe” COVID-19 pneumonia defined as patients who cannot saturate > 96% on 4 L/min of O₂ and who are neither on ‘non-invasive’ ventilation, NOR invasive mechanical ventilation NOR ECMO. The primary composite end point is the rate of use of mechanical ventilation (i.e., need for intubation) or death at 15 days after inclusion. The secondary endpoints for the study are:
Clinical status evolution at day 7, 15 and 30 as assessed by the 9-point ordinal scale
Survival at day 7, 15 and 30
Time to clinical improvement, defined as time from randomization to either an improvement of 1 point on the 9-point ordinal scale or discharge from hospital
Duration of hospitalization and ICU stay

Explorative (research) endpoints:
Quantitative PCR SARS-CoV-2 virus in nasopharyngeal samples (time frame: before MSC infusion on Day 0 and after MSC infusion as per clinical indication).
White blood cells, hemoglobin, platelets count and standard hemostasis markers (D-dimer levels, prothrombin time, International Normalized Ratio (INR), fibrinogen and antiXa activity), assessed in peripheral blood (time frame: before MSC infusion on Day 0 and after MSC infusion on Days 1, 3, 5, 8, 15, and on Day 30 or Day of hospital discharge if earlier).
Standard biological markers of inflammation and myocardial damage: glutamic oxaloacetic and alanine aminotransferase, total bilirubin, serum creatinine, blood electrolytes, creatine phosphokinase, NT-proBNP, myoglobin, haptoglobin, Troponin T, procalcitonin, LDH, ferritin, CRP, assessed in peripheral blood (time frame: before MSC infusion on Day 0 and after MSC infusion on Days 1, 3, 5, 8, 15, and on Day 30 or Day of hospital discharge if earlier).
Lung damage assessed by imaging techniques (Chest X ray/CT scan /or on doppler ultrasound) when clinically indicated
Research biomarkers including Angiopoietin 2, IL-1β, IL-6, IL-8, IL-10, TNFα, VEGF, RAGE and antiSARS-CoV2 IgG and IgA; evaluated using multiplex electrochemiluminescence based techniques. Change from Baseline (time frame: immediately before MSC infusion on Day 0, and after MSC infusion at 3 hours, and on days 1, 8, 15, and on Day 30 or Day of hospital discharge if earlier).
Peripheral blood mononuclear cells for single cell RNA Sequencing and/or flow cytometry (time frame: immediately before MSC infusion on Day 0, and after MSC infusion at 3 hours, and on days 1, 8, and on Day 30 or Day of hospital discharge if earlier).

Study design: This is a randomised, double-blinded placebo controlled Phase II Clinical Trial. A total of 48 patients will be enrolled, with a 1:1 randomisation of Final Product to Placebo used. Stratification of the study population to each treatment arm will be based on age and gender. Each patient enrolled will receive an intravenous infusion of Final Product or Placebo and evaluated for 12 months post-treatment.

Screening and Informed Consent: After signature of the informed consent, the following assessments will be performed to determine eligibility requirements as specified in the inclusion and exclusion criteria, and will be scheduled before the inclusion of the patient in the study:
Demography, medical history (treatments, co-morbidities and allergies)
Confirm the positive SARS-CoV-2 test result by PCR in any specimen before randomization
Classification of the pneumonia, assessed by SpO₂ measurement and need of supplemental oxygen
Blood tests for screening laboratory evaluations, if last results <72 h from day of randomization:
   Absolute neutrophil count
   Platelets
   ALT or AST
   Creatinine
   Serological profile of VIH, syphilis, VHB, VHC
Investigate ongoing tuberculosis infection
Urinary test if last results <72 h from day of randomization
Urine or serum pregnancy test (in women of childbearing potential)

The following assessments will be performed as needed for baseline data, in addition to already-obtained screening measures:
At Day 0, prior to MSC infusion:
Clinical status as assessed on the 9-point ordinal scale (0. No clinical or virological evidence of infection. 1. No limitations of activities. 2. Ambulatory, limitation of activities. 3. Hospitalized, not requiring supplemental oxygen. 4. Hospitalized, requiring supplemental oxygen. 5. Hospitalized, on non-invasive ventilation or high flow oxygen devices. 6. Hospitalized, intubated and on mechanical ventilation. 7. Hospitalized, ventilation + additional organ support - pressors, RRT, ECMO, 8. Death)
Quantitative PCR SARS-CoV-2 virus in nasopharyngeal swabs
O₂ saturation and O₂ requirement
White blood cell, hemoglobin, platelets count and standard hemostasis markers (D-dimer levels, prothrombin time, INR, fibrinogen, and antiXa activity) in peripheral blood
Standard biological markers for inflammation and myocardial damage within the peripheral blood: ALT, AST, total bilirubin, serum creatinine, blood electrolytes, creatine phosphokinase, NT-proBNP, myoglobin, haptoglobin, Troponin T, procalcitonin, LDH, ferritin, CRP
Plasma and Peripheral blood mononuclear cell (PBMC) biobanking

Any time prior to MSC infusion, for COVID-19 evaluation Chest X ray/CT scan /or on doppler ultrasound if done for clinical purposes

MSC Infusion: Cryobags are thawed at bedside and diluted in 100 ml of saline prior to administration. Cells will be delivered at a rate of 5 million cells per minute over a total of 20 minutes. The product will be evaluated visually to ensure no visible clumps or cell debris prior to, or during, the infusion process.

Experimental treatment group: single dose of 100×10⁶ WJ-MSC/patient administered by slow intravenous infusion at a rate of 5 million cells per minute over a total of 20 minutes. Placebo group: sodium chloride buffer supplemented with 5 % human serum albumin (HSA) and 10 % dimethyl sulphoxide (DMSO) same volume and mode of administration than the experimental treatment group.

Follow-up visits: The following assessments will be performed daily after the intervention
Clinical status as assessed on the 9-point ordinal scale
O₂ saturation and O₂ requirement evaluation twice a day. Worst value will be taken
Evaluation of the safety according to the criteria of the Common Terminology Criteria for Adverse Events (CTCAE) Version 5
The following evaluations will be performed at Day 1, 3, 5, 8, 15 and 30 (or day of hospital discharge if earlier). If they are done as part of clinical care the data will be included in the study, otherwise samples will be obtained, stored and tested as part of ‘exploratory outcomes:
Biological examinations including:
   Hematology: White Blood Cells, hemoglobin, platelet count
   Biochemistry: ALT, AST, total bilirubin, serum creatinine, blood electrolytes, CPK, NT-proBNP, myoglobin, haptoglobin, Troponin T, procalcitonin, LDH, ferritin, CRP
   Hemostasis markers: D-dimer levels, prothrombin time, INR, fibrinogen, and
   antiXa activity
Plasma and PBMC biobanking will be performed at Day 0, 3 hours after MSC infusion, and days 1, 8, and Day 30 (or day of hospital discharge if earlier)
Evaluation of survival will be assessed at Day 7, 15 and 30
Chest X ray/CT scan or doppler ultrasound if clinically indicated

Final study visit: The End of the study is when all inclusions and all patients have completed their follow-ups. The last visit of this study is Day 30. If patient is discharged from the hospital before day 30, he/she will undergo last biological evaluations at day of discharge but clinical status since hospital discharge and adverse events will be assessed by phone call to the patient at weekly intervals.

### Example 8

The present Example describes the selection criteria for the study population. Each patient enrolled in the study has to fulfill all inclusion criteria and none of the exclusion criteria.

Inclusion and Exclusion criteria: Subjects will be recruited from the population of diagnosed with COVID-19.

Inclusion criteria are as follows:
Male or female, ≥18 years old
Has laboratory-confirmed SARS-CoV-2 infection as determined by reverse-transcription polymerase chain reaction (RT-PCR) in any specimen prior to inclusion.
Hospitalized patients
Patients classified as severe pneumonia, as defined by the need for continuous supplemental oxygen 5 L/min O₂ OR high flow oxygen, 35% FiO₂ > 301 /min and cannot saturate > 96% NOT under “non-invasive” ventilation NOR invasive mechanical ventilation NOR ECMO
Women of childbearing potential must agree to use contraception or acceptable birth control for the duration of the study.

Exclusion criteria are as follows:
Inability to provide informed consent
Patients not expected to survive for 24 hours or mechanically ventilated at inclusion or previously during present hospitalization
Pregnant or nursing (lactating) women, where pregnancy is defined as the state of a female after conception and until the termination of gestation, confirmed by a positive human chorionic gonadotrophin (hCG) laboratory test
Patients with BMI ≥30
Patients with known, or previous, malignancy
Patients with other serious systemic diseases deemed of contra-indication by the physician
Patient with any of following laboratory results out of the ranges detailed below at screening: Absolute neutrophil count (ANC) ≤ 1.0 × 10⁹/L, Platelets (PLT) < 50 10⁹ /L, ASAT or ALAT > 5N, eGFR < 30 mL/min
Current documented bacterial infection
Serological evidence of infection with human immunodeficiency virus, Treponema pallidum, hepatitis B antigen (serology consistent with previous vaccination and a history of vaccination is acceptable) or hepatitis C
Latent or previous as well as on-going therapy against tuberculosis, or exposed to tuberculosis or have travelled in areas with high risk of tuberculosis or mycosis within the last 3 months
Patients with known allergies to a component of the ProTrans® product
Ongoing treatment with Remdesivir
Pre-existing chronic respiratory diseases requiring long- term oxygen therapy
Pre-existing cirrhosis with basal Child and Pugh of C
Patients with history of increased risk for thrombo- embolic and/or co-morbidity for thrombo- embolism
Patients with a history of myocardium infarction
A history of cardiac dysfunction¹

### Results

Study population of 9 individuals is selected based on the criteria described above. A subject may withdraw consent for study participation either before or after administration of the trial intervention. The reason for subject discontinuation will be documented in the Case Report Form (CRF). If a subject is discontinued due to an AE, the nature of the event and its clinical course must be fully documented

### Example 9

The present Example describes how the clinical study is performed. Herein the inventive pooled allogeneic MSC composition is referred to as Drug Product and the pharmaceutical composition as Final Product. Thus, the Final Product comprises the Drug Product.

### Material and Methods

The Drug Product is defined as an allogeneic cell suspension of from multiple donors. MSC are isolated through explant from Wharton Jelly, expanded until passage 2 or 3. The Drug Product contains pooled ex vivo expanded cells from 5 donors. The production of each batch starts with explant culture of tissue from approximately 20 qualified donors from which 5 donors are finally selected as Drug Product donors as described herein.. In addition to the MSC characterization, cells are selected based on morphology, proliferative capacity and functional assays related to immunosuppression and immunomodulatory capacity.

The cells are frozen in cryo bags at concentrations of 30, 60 or 100 × 10⁶ cells in 5 ml 5% HSA and 10% DMSO, one cryo bag contains one dose. The bags are frozen in a controlled rate freezer and directly transferred to -190° C. for storage until it is time for infusion. The cryopreserved bags are transported by the Manufacturer on liquid Nitrogen to the investigator’s site, where it is thawed bed-side and diluted in autologous spinal fluid which is aspirated through lumbar puncture for immediate dilution of drug product and intrathecal injection. Alternatively, where intravenous or intraarterial infusion will be utilized the Drug Product will be diluted in 100 ml of saline prior to administration. Minimum 24 hours before injection, the investigator will send a requisition to the Manufacturer for delivery of the IP. On the day of infusion, the applicable Final product is transported by the Manufacturer to the investigator site.

The Final Product is considered delivered when the Manufacturer has handed over the cryobag in liquid nitrogen transportation canister. When the patient is ready for infusion, the cells are thawed bed-side in a water bath. The thawed Final Product is diluted in 10 ml spinal fluid which gives a total of 15 ml infusion volume for intrathecal delivery or 100 ml of saline for intravenous or intraarterial delivery. The Final Product should be administered to the patient within 30 minutes after preparation, however not later than 2 hours after preparation..

Cells are administered intravenously in a dose of 100 ml of saline. The placebo will be an equivalent volume DMSO for intravenous or intraarterial studies. For phase II studies this will be a double-blinded study where both the study subjects and study personnel performing post-injection assessments of safety and efficacy will be blinded to whether subjects received WJMSC or placebo.

Subjects will be randomised to receive either Drug Product or Placebo treatment.

All patients will receive standard COVID-19 treatment. Study patient receiving concomitant medication which may interfere with study treatment will be withdrawn from the study. The investigator will instruct the patient to notify the study site about any new medications he/she is taking when study treatment has started. All medications and significant non-drug therapies (including physical therapy and blood transfusions) administered after the patient starts treatment with study drug must be listed in the CRF and medical records.

The study is completed at the 2 year follow-up after infusion of Drug Product/Final Product.

### Results

The above described approach assures the proper application of the product and allows the study of the safety and efficacy of the product.

Number and frequency of adverse events will be recorded from the time of enrolment until the end of the follow-up period or, in the case of early withdrawal, to the time of study withdrawal. Adverse Events (AEs and SAEs) are noted in the patient’s medical record and a separate AE/SAE report is completed for each AE/SAE. Patients will be asked to report any adverse events at each visit following the screening visit. Vital signs, physical examination, neurological examination (by investigator), blood and urine samples will be analysed at specified times throughout the study for safety.

Two patients have received an infusion with 25 million cells formulated as investigational medicinal product IMP (in other words Final Product) in the clinical trial EudraCT number: 2020-002078-29 (Treatment of Respiratory Complications Associated with COVID-19 Infection Using Wharton’s Jelly (WJ)-Umbilical Cord (UC) Mesenchymal Stromal Cells (ProTrans®): open Phase IB Clinical Trial). No servere adverse reactions has been registered and the patients were discharged 1 and 5 days after infusion, respectively. One patient has received an infusion with either 100 million cells formulated as IMP or placebo in the Canadian clinical trial Project code number: 2021-6954 (Treatment of Respiratory Complications Associated with COVID-19 Infection Using Wharton’s Jelly (WJ)-Umbilical Cord (UC) Mesenchymal Stromal Cells (ProTrans®): a Randomized Phase II Controlled Clinical Trial). No severe adverse reactions has been registered.

It is expected that the present example will show that treatment with Drug Product as disclosed herein will show one or more of the following results: decrease in hospitalization time, decreased need for supplemental oxygen or high flow supplemental oxygen, decreased need for medical ventilation (such as need for mechanical ventilation for example ECMO), decreased need for organ support, and increase survival.

Additionally, it is expected that the present treatment will not lead to any clinically relevant induction of anti-HLA antibodies in the patients

### Example 10

The present Example shows that the isolated, pooled allogeneic MSC population according to the present disclosure exhibit higher baseline secretion of immunomodulatory molecules, without the need for culture post-pooling, compared to MSCs derived from single donors or other sources of MSCs such as bone marrow MSCs.

### Material and Methods

Assay 1: IDO assay. IDO assay is used to analyze the immunosuppressive capacity of Drug Substance Intermediate or Drug Substance, i.e. mesenchymal stem/stromal cells (MSC).

The WJ-MSC immunomodulatory potential is reported as a measure of indoleamine 2,3-dioxygenase (IDO) activity, determined by measuring tryptophan and kynurenine in the culture supernatant. The IDO activity is the ratio of kynurenine/tryptophan and can be determined by calculating the amount of tryptophan and kynurenine present in cell culture supernatants using an ELISA kit. The inventors present data demonstrating that pooling of WJ-MSCs results in higher baseline (unstimulated) levels of IDO activity compared to single WJ-MSC donors or bone marrow derived MSCs.

MSC culturing: Seed 10 000 MSC / well in 48-well cell culture plates in 100 µl assay medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)). Add 100 µl assay medium to the cells.

Incubate cell culture plate at 37° C., 5% CO₂ for 72 hours. Remove the supernatant from each well and store in micro tubes at -20° C. until further processing for ELISA analysis. Tryptophan and kynurenine measurements are done according to manuals provided by the ELISA-kit manufacturer (Immundiagnostik AG, cat no. K 3730 and K 3728). Both tryptophan and kynurenine ELISA are performed on the same day but at separate occasions. The two ELISAs are conducted according to manufacturer’s instructions; see the manuals for respective ELISA.

Absorption at 450 nm with background subtraction at 620 nm is measured in a Spectramax microplate reader (Molecular Devices, Spectramax 190).

Analyzing results: Amount of absorbance measured is inversely proportional to the amount of amino acid present in the sample; i.e. the lower the OD450, the more kynurenine or tryptophan there is. The 4PL-algorithm (Four Parameter Logistic Regression) is used to calculate results (software SoftMax Pro 7.0.2, Molecular Devices), as recommended by kit manufacturer. Concentrations are determined directly from the standard curve. The control samples provided with the kits should are evaluated for acceptability: if outside the acceptable range according to the manufacturer of the kit, the samples need to be re-assayed.

### Results

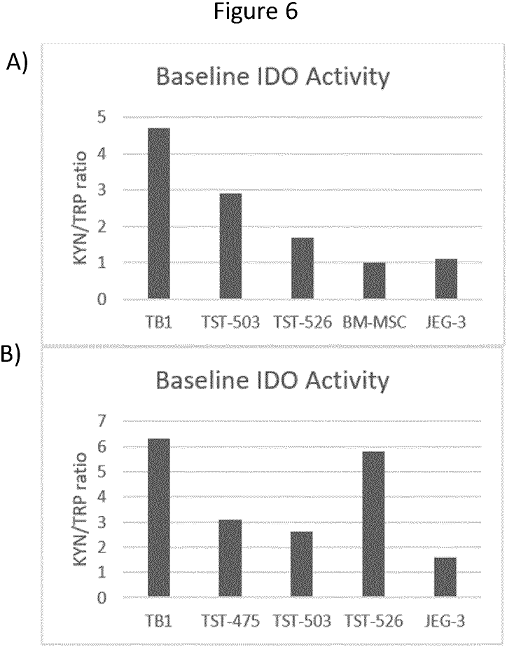

The ratio of kynurenine/tryptophan was evaluated in pooled WJ-MSCs (TB1) compared to single cell WJ-MSC donors, bone marrow derived MSCs and a JEG-3 control cell line derived from human placenta choriocarcinoma. Higher baseline IDO activity was seen in TB1 pooled WJ-MSCs compared to all other cell sources evaluated (FIG. 6).

### Material and Methods

Assay 2: Prostaglandin E2 (PGE2) assay evaluates Drug Substance Intermediate and/or Drug Substance secretion of PGE2 in culture medium supernatant.

Cell culturing: Cells are cultured in assay medium (DMEM, low glucose, GlutaMAX™ Supplement, pyruvate (ThermoFisher Scientific, cat no. 21885025) + 10% Fetal Bovine Serum, qualified, heat inactivated (ThermoFisher Scientific, cat no. 16140071)) for 3 days. 40 000 MSCs are seeded per well in 12-well cell culture plates. Cell culture plates are incubated at 37° C., 5% CO₂.500 µl assay medium. The cell culture plate is incubated at 37° C., 5% CO₂ for 72 hours. The supernatant is removed from each well and centrifuged for 5 min at 500 g to remove particulates. The supernatant is frozen and stored at -20° C. until further processing for ELISA analysis.

The Parameter™ Prostaglandin E2 Immunoassay kit is used for PGE2 expression detection according to manufacturer’s instruction (Bio-Techne, cat no. KGE004B) and is analyzed with Spectramax microplate reader (Molecular Devices, Spectramax 190). The 4PL-algorithm (Four Parameter Logistic Regression) is used to calculate results (software SoftMax Pro 7.0.2, Molecular Devices).

### Results

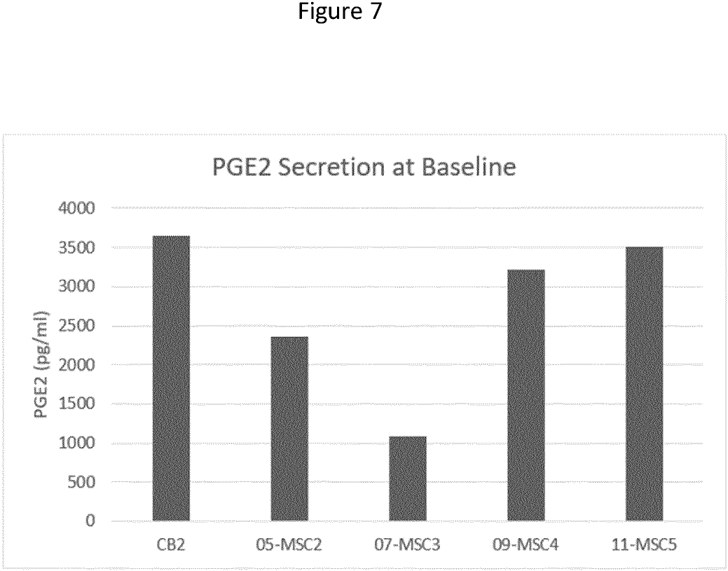

Levels of PGE2 secretion by pooled WJ-MSCs (TB1) and single donors was evaluated over 72 hours. Baseline levels of secretion of PGE2 were higher in pooled cells compared to the single donors (FIG. 7).

### ITEMIZED LIST OF EMBODIMENTS

1. Isolated, pooled allogeneic mesenchymal stem cell (MSC) population for use in the treatment and/or prevention of COVID-19 infection or for use in the treatment and/or prevention of symptoms associated with COVID-19 infection, wherein said isolated, pooled allogeneic MSC population comprises MSCs derived from at least 3 individual donors, wherein the number of cells derived from any one donor does not exceed 50% of the total cell number and wherein said MSCs have at most been subject to ten passages and wherein said isolated, pooled allogeneic MSC population is obtainable by a method comprising the steps of:
culturing or providing MSCs from more than said at least 3 individual donors to obtain more than at least 3 individual donor derived MSC populations;
assaying each individual donor derived MSC population using at least 3 assays to obtain at least 3 assay results for said each individual donor derived MSC population;
for each assay allocating an individual ranking score value to said each individual donor derived MSC population based on the assay result and thus obtaining at least 3 individual ranking score values for each individual donor derived MSC population, wherein a higher ranking score value is indicative of more desirable assay result; or wherein a lower ranking score value is indicative of more desirable assay result;
allocating a total score value to each individual donor derived MSC population based on said at least 3 individual ranking score values, wherein in the case of a higher ranking score value being indicative of more desirable assay result, a higher total score value is indicative of more desirable population properties; or wherein in the case of a lower ranking score value being indicative of more desirable assay result, a lower total score value is indicative of more desirable population properties;
selecting a subset of individual donor derived MSC populations with desirable population properties based on their total score values; and
pooling said selected individual donor derived MSC populations to obtain an isolated, pooled allogeneic MSC population;
wherein at least 2 of said at least 3 assays are selected from the group consisting of one assay measuring indoleamine-2,3-dioxygensase (IDO) activity; one assay measuring prostaglandin E2 secreted by said MSCs; and
one assay measuring the effect of said MSCs on the proliferation of peripheral blood mononuclear cells (PBMCs) and
wherein at least 1 of said at least 3 assays is selected from the group consisting of one assay measuring the effect of said MSCs on the capacity of T cells to suppress an immune response; one assay measuring the effect said MCSs on the proliferation and/or apoptosis of dendritic cells, one assay measuring the effect of the said MSCS on monocytes; and one assay measuring the effect of the said MSCs on microglia cell and/or microglia-like cells.

2. Isolated, pooled allogeneic MSC population for use according to item 1, wherein said pooled allogeneic MSC population is not further cultured after the pooling step.

3. Isolated, pooled allogeneic MSC population for use according to item 1 or 2, wherein the individual ranking score value for at least one assay is allocated to said each individual donor derived MSC population based on a comparison of the assay result for said each individual donor derived MSC population to the results for the remaining individual donor derived MSC populations.

4. Isolated, pooled allogeneic MSC population for use according to any one of items 1-3, wherein the individual ranking score value for at least one assay is allocated to said each individual donor derived MSC population based on absolute assay result obtained for said individual donor derived MSC population.

5. Isolated, pooled allogeneic MSC population for use according to item 4, wherein the assay result is deemed desirable and an individual ranking score value that reflects the obtained desirable assay result is allocated, when said absolute result corresponds to at least a predetermined value or at most a predetermined value.

6. Isolated, pooled allogeneic MSC population for use according to any one of items 1-5, wherein the step of selecting a subset of individual donor derived MSC populations with desirable population properties comprises selecting the individual donor derived MSC populations with total score value which corresponds to at least a predetermined value in the case wherein a higher total score value is indicative of more desirable population properties; or to at most a predetermined value in the case wherein a lower total score value is indicative of more desirable population properties.

7. Isolated, pooled allogeneic MSC population for use according to any one of items 1-5, wherein the step of selecting a subset of individual donor derived MSC populations with desirable population properties comprises selecting a predetermined number of the individual donor derived MSC populations, which populations exhibit a higher total score value relative the remaining individual donor derived MSC populations in the case wherein a higher total score value is indicative of more desirable population properties; or which populations exhibit a lower total score value relative the remaining individual donor derived MSC populations in the case wherein a lower total score value is indicative of more desirable population properties.

8. Isolated, pooled allogeneic MSC population for use according to any one of item 1-7, wherein said MSCs have at most been subject to seven passages, such as at most six passages, such as at most five passages, such as at most four passages, such as at most three passages, such as one, two or three passages, such as two or three passages.

9. Isolated, pooled allogeneic MSC population for use according to any one of items 1-8, wherein said MSCs are derived from native MSC source.

10. Isolated, pooled allogeneic MSC population for use according to any one of items 1-9, wherein said MSCs are selected from the group consisting of bone marrow derived MSCs, peripheral blood derived MSCs, adipose tissue derived MSCs, dental tissue derived MSCs, oral mucosal derived MSCs, placenta derived MSCs, umbilical cord derived MSCs, amniotic fluid derived MSC, cord blood derived MSCs, Wharton Jelly derived MSCs, decidua derived MSCs, chondrion membrane derived MSCs and amnion membrane derived MSCs; such as the group consisting of placenta derived MSCs, umbilical cord derived MSCs, amniotic fluid derived MSC, cord blood derived MSCs, Wharton Jelly derived MSCs, decidua derived MSCs, chondroid membrane derived MSCs, dental pulp derived MSCs and amnion membrane derived MSCs.

11. Isolated, pooled allogeneic MSC population for use according to item 10, wherein said MSCs are selected from the group consisting of umbilical cord derived MSCs and Wharton Jelly derived MSCs, such as wherein said MSCs are Wharton Jelly derived MSCs.

12. Isolated, pooled allogeneic MSC population for use according to any one of items 1-11, wherein said population comprises MSCs derived from at least four individual donors, such as at least five individual donors, such as at least six individual donors, such as at least seven individual donors, such as at least eight individual donors, such as at nine individual donors, such as at least ten individual donors.

13. Isolated, pooled allogeneic MSC population for use according to any one of items 1-12, wherein said population comprises MSCs derived from 3-20 individual donors, such as 3-15 individual donors, such as 3-10 individual donors, such as 4-8 individual donors, such as 5-7 individual donors, such as 5, 6 or 7 individual donors.

14. Isolated, pooled allogeneic MSC population for use according to any one of items 1-13, wherein the step of assaying each individual donor derived MSC population comprises assaying at least 1-4 times, such as 2-4 times, such as 2-3 or 3-4 times, as many individual donor derived MSC population as the number of individual donor derived MSC populations pooled in the pooling step.

15. Isolated, pooled allogeneic MSC population for use according to any one of items 1-14, wherein the step of assaying each individual donor derived MSC population comprises assaying at least 3, such as at least 4, such as at least 5, such as at least 6, such as at least 7, such as at least 8, such as at least 9, such as at least 10, such as at least 11, such as at least 12, such as at least 13, such as at least 14, such as at least 15, such as at least 16, such as at least 17, such as at least 18, such as at least 19, such as at least 20 individual donor derived MSC populations.

16. Isolated, pooled allogeneic MSC population for use according to any one of items 1-14, wherein the step of assaying each individual donor derived MSC population comprises assaying 3-50 individual donor derived MSC populations, such as 4-50, such as 5-50, such as 6-50, such as 6-30, such as 6-20, such as 6-15, such as 8-12 individual donor derived MSC population.

17. Isolated, pooled allogeneic MSC population for use according to any one of items 1-16, the step assaying each individual donor derived MSC population using at least 3 assays comprises using as least one functional assay, such as at least two functional assays, such as at least three functional assays, such at least four functional assays, such least five functional assays.

18. Isolated, pooled allogeneic MSC population for use according to any one of items 1-17, wherein said at least one assay measuring IDO activity comprises of the step of measuring IDO activity within the culture supernatant of MSCs co-cultured with stimulated PBMCs or purified T cells or activated monocytes/macrophages or microglia.

19. Isolated, pooled allogeneic MSC population for use according to any one of items 1-18, wherein said at least one assay measuring prostaglandin E2 secreted by said MSCs comprises measuring prostaglandin E2 secreted by said MSCs when co-cultured with PBMCs, such as phytohaemagglutinin (PHA) stimulated PBMCs, such as PHA stimulated T-lymphocytes or co-cultured with interferon γ and/or tumor necrosis factor alpha.

20. Isolated, pooled allogeneic MSC population for use according to any one of item 1-19, wherein said proliferation of PBMCs is the proliferation of T-lymphocytes, such as proliferation of phytohaemagglutinin (PHA) stimulated T-lymphocytes.

21. Isolated, pooled allogeneic MSC population for use according to any one of item 1-20, wherein said at least 1 of said at least 3 assays isone assay measuring the effect of said MCSs on the capacity of T cells to suppress an immune response.

22. Isolated, pooled allogeneic MSC population for use according to any one of item 1-21, wherein said at least 1 of said at least 3 assays is one assay measuring the effect said MSCs on the proliferation and/or apoptosis of dendritic cells or one assay measuring the effect said MSCs on inducing tolerogenic dendritic cells.

23. Isolated, pooled allogeneic MSC population for use according to any one of item 1-22, wherein said at least 1 of said at least 3 assays is one assay measuring the effect of the said MSCs on microglia cells or microglia-like cells is selected from the group consisting of one assay measuring microglial proliferation; one assay measuring expression of markers characteristic of the M1 phenotype in microglia; one assay measuring expression of markers characteristic of the M2 phenotype in microglia; and an assay measuring the shift from the M1 microglia phenotype to the M2 microglia phenotype.

24. Isolated, pooled allogeneic MSC population for use according to item 23, wherein said one assay measuring microglial proliferation comprises cocultivation of said individual donor derived MSC population(s) with microglia cells and/or microglia-like cells.

25. Isolated, pooled allogeneic MSC population for use according to item 23 or 24, wherein said microglia cells or microglia-like cells are selected from the group consisting of immortalized cell lines, such as the human microglial HMC3 cell line or the CHME-5 cell line; primary microglia obtained from biopsies; primary microglia-like cells cultured from cord blood; and immortalized microglia-like cells from cord blood, such as the DUOC-01 cell line; such as selected from the group consisting of the consisting of immortalized cell lines, for example selected from the group consisting of HMC3 cell line, CHME-5 cell line and the DUOC-01 cell line.

26. Isolated, pooled allogeneic MSC population for use according to any one of items 23-25, wherein said one assay measuring microglial proliferation comprises assaying if a decrease in the proliferation microglia cells occurs upon mitogen stimulation, such as lipopolysaccharide stimulation, or quantifying a decrease in the proliferation microglia cells upon lipopolysaccharide stimulation.

27. Isolated, pooled allogeneic MSC population for use according to any one of items 23-26, wherein said proliferation is measured as a proliferation percentage, is measured as a proliferation index or is measured as a growth index, such as is measured as a growth index.

28. Isolated, pooled allogeneic MSC population for use according to any one of items 23-27, wherein said one assay measuring expression of markers characteristic of the M1 phenotype in microglia and/or microglia-like cells comprises measuring the expression of at least one marker selected from the group consisting of CD183, CD11b, CD14, B7-2/CD86, Integrin alpha V beta 3, MFG-E8, NO, ROS, RNS, CCL2/MCP-1, CCL3/MIP-1 alpha, CCL4/MIP-1 beta, CCL5/RANTES, CCL8/MCP-2, CCL11/Eotaxin, CCL12/MCP-5, CCL15/MIP-1 delta, CCL19/MIP-3 beta, CCL20/MIP-3 alpha, CXCL1/GRO alpha/KC/CINC-1, CXCL9/MIG, CXCL10/IP-10, CXCL11/I-TAC, CXCL13/BLC/BCA-1, CX3CL1/Fractalkine, MMP-3, MMP-9, Glutamate, IL-1 beta/IL-1F2, IL-2, IL-6, IL-12, IL-15, IL-17/IL-17A, IL-18/IL-1F4, IL-23, IFNγ, TNF-alpha, Fc gamma RIII/CD16, Fc gamma RII/CD32, CD36/SR-B3, CD40, CD68/SR-D1, B7-1/CD80, MHC II, iNOS and COX-2; such as at least one marker selected from the group consisting of CD183, CD11b and CD14.

29. Isolated, pooled allogeneic MSC population for use according to item 28, wherein said one assay measuring expression of markers characteristic of the M1 phenotype in microglia and/or microglia-like cells comprises measuring the expression of at least CD183.

30. Isolated, pooled allogeneic MSC population for use according to any one of items 28-29, wherein a decrease in expression of at least one of the markers whose expression in measured by said one assay measuring expression of markers characteristic of the M1 phenotype in microglia and/or microglia-like cells is indicative of a desirable result.

31. Isolated, pooled allogeneic MSC population for use according to item 23-27, wherein said one assay measuring expression of markers characteristic of the M2 phenotype in microglia and/or microglia-like cells comprises measuring the expression of at least one marker selected from the group consisting of CX3CR1, CD200R, CD206, IL-1ra/IL-1F3, IL-4, IL-10, IL-13, TGF-beta, CCL13/MCP-4, CCL14, CCL17/TARC, CCL18/PARC, CCL22/MDC, CCL23/MPIF-1, CCL24/Eotaxin-2/MPIF-2, CCL26/Eotaxin-3, FlZZ1/RELM alpha, YM1/Chitinase 3-like 3, CLEC10A/CD301, MMR/CD206, SR-Al/MSR, CD163, Arginase 1/ARG1, Transglutaminase 2/TGM2, PPAR and gamma/NR1C3; such as at least one marker selected from the group consisting of CX3CR1, CD200R and CD206.

32. Isolated, pooled allogeneic MSC population for use according to item 31, wherein said one assay measuring expression of markers characteristic of the M2 phenotype in microglia and/or microglia-like cells comprises measuring the expression of at least CD200R.

33. Isolated, pooled allogeneic MSC population for use according to any one of items 31-32, wherein an increase in expression of at least one of the markers whose expression in measured by said one assay measuring expression of markers characteristic of the M2 phenotype in microglia and/or microglia-like cells is indicative of a desirable result.

34. Isolated, pooled allogeneic MSC population for use according to any one of items 28-33, wherein said shift from the M1 microglia phenotype to the M2 microglia phenotype is measured as a decrease in the expression of any one or more of the markers defined in any one of items 28-29 and an increase in the expression of any one or more of the markers defined in any one of items 31-32.

35. Isolated, pooled allogeneic MSC population for use according to item 34, wherein said shift from the M1 microglia phenotype to the M2 microglia phenotype is measured as a decrease in the expression of any one or more of the markers selected from CD183, CD11b and CD14 and an increase in the expression of any one or more of the markers selected from CX3CR1, CD200R and CD206, such as wherein said shift from the M1 microglia phenotype to the M2 microglia phenotype is measured as a decrease in the expression of CD183 and an increase in the expression of CD200R.

36. Isolated, pooled allogeneic MSC population for use according to any one of items 34-35, wherein said shift from the M1 microglia phenotype to the M2 microglia phenotype is indicative of a desirable result.

37. Isolated, pooled allogeneic MSC population for use according to any one of items 1-36, wherein said one assay measuring the effect of the said MSCs on monocytes comprises measuring the shift from classical to non-classical monocyte phenotype in response to said MSCs, such as measures the effect of said MSC on monocyte shift towards regenerative phenotype.

38. Isolated, pooled allogeneic MSC population for use according to any one of items 1-37, wherein said at least 3 assays further comprise at least one assay measuring HLA-G expression in said MSCs in response to IFNγ, tumor necrosis factor alpha, alum, IL-10, PHA and/or GABA, such as in response to IFNγ, IL-10 and/or PHA.

39. Isolated, pooled allogeneic MSC population for use according to any one of items 1-38, wherein said at least 3 assay further comprise at least one assay measuring the protein expression and/or cytokine expression.

40. Isolated, pooled allogeneic MSC population for use according to item 39, wherein said at least one assay measuring the protein expression and/or cytokine expression measures the expression of one or several proteins or cytokines selected from the group consisting of IL-2, IL-4, IL-6, IL-8, IL-12, IL-12/13, IL17A, IL-21, IL-22, IL-29, IL-31, TGFβ, VEGF, FGF, GM-CSF, IFNα, IFNγ, apo E and TNFα, such as the group consisting of IL-6, IL-8, GM-CSF and TGFβ, such as the group consisting of at least IL-6 or wherein said at least one assay measuring the protein expression and/or cytokine expression measures one or several proteins or cytokines selected from the group consisting of IL-2, IL-4, IL-6, IL-8, IL-12, IL- 12/13, IL17A, IL-21, IL-22, IL-29, IL-31, TGFβ, VEGF, FGF, GM-CSF, IFNα, IFNγ, apo E and TNFα and ACE2 receptor and TMPRSS2, such as the group consisting of IL-6, IL-8, GM-CSF,TGFβ, ACE2 receptor and TMPRSS2, such as the group consisting of at least ACE2 receptor and TMPRSS2.

41. Isolated, pooled allogeneic MSC population for use according to item 40, wherein the expression of at least 2, such as at least 3, such as at least 4, such as at least 5, such as at least 6, such as at least 7, such as at least 8, such as at least 9, such as at least 10, such as at least 11, such as at least 12, such as at least 13, such as at least 14, such as at least 15, such as at least 16, such as at least 17, such as at least 18, such as all 19 of said proteins and/or cytokines are measured.

42. Isolated, pooled allogeneic MSC population for use according to any one of items 39-41, wherein said expression is measured in absence and/or presence of at least one stimuli.

43. Isolated, pooled allogeneic MSC population for use according to item 42, wherein said stimuli is an immune response modifying stimuli.

44. Isolated, pooled allogeneic MSC population for use according to item 43, wherein said immune response modifying stimuli is selected from the group consisting of PBMCs; stimulated PBMCs, such as PBMCs stimulated with PHA, IL10, gamma-aminobutyric acid (GABA), anti-CD2, anti-CD3, anti-CD28, alum and/or interferon gamma (IFNγ).

45. Isolated, pooled allogeneic MSC population for use according to item 43 or 44, wherein said immune response modifying stimuli is gamma-aminobutyric acid (GABA) or wherein said immune response modifying stimuli is PBMCs stimulated with gamma-aminobutyric acid (GABA).

46. Isolated, pooled allogeneic MSC population for use according to item 42-44, wherein said stimuli is a cytokine, such as interferon gamma (IFNγ).

47. Isolated, pooled allogeneic MSC population for use according to item any one of items 42-46, wherein stimuli is selected from the group consisting of polyinosinic:polycytidylic acid (Poly I:C), resiquimod (r848), gamma-aminobutyric acid (GABA) and IFNγ, such as the group consisting of Poly I:C and IFNγ.

48. Isolated, pooled allogeneic MSC population for use according to item any one of items 42-44, wherein stimuli is PBMCs, such as stimulated or unstimulated PBMCs, such as PHA stimulated PBMCs, such as PHA stimulated T-lymphocytes.

49. Isolated, pooled allogeneic MSC population for use according to any one of items 1-48, wherein said at least 3 assays comprises at least one morphological assay.

50. Isolated, pooled allogeneic MSC population for use according to item 49, wherein said morphological assay assays morphological features of cells and/or cells nuclei.

51. Isolated, pooled allogeneic MSC population for use according to item 50, wherein said morphological features of cells and/or cells nuclei are one or more features selected from the group consisting of the size of the cell, the size of the nuclei, the shape of the cell and the ratio between cell and nuclei size.

52. Isolated, pooled allogeneic MSC population for use according to any one of items 49-51, wherein an individual donor derived MSC population is only eligible for pooling if it exhibits more than or equal to 90%, such as at least 91%, such as at least 92%, such as at least 93%, such as at least 94%, such as at least 95%, such as at least 96%, such as at least 97%, such as at least 98%, such at least 99% normal cells and/or nuclei.

53. Isolated, pooled allogeneic MSC population for use according to any one of items 1-52, wherein the step of assaying each individual donor derived MSC population using at least 3 assays is performed when the MSC population is in passage 0 (p0) - passage 8 (p8), such as in p1 - p 5, such as in p1 - p4, such as in p2 - p4 or in p1 - p4, such as in p1, p2 and/or p3, such as in p2 and/or p3.

54. Isolated, pooled allogeneic MSC population for use according to any one of items 1-53, at least one assay, such as at least two assays, such as at least three assays, such as all assays, is/are performed when the cells are in the same passage as when they are pooled.

55. Isolated, pooled allogeneic MSC population for use according to any one of items 1-53, wherein at least two assays are performed at different passages.

56. Isolated, pooled allogeneic MSC population for use according to any one of items 1-55, wherein said total score value allocated to said each individual donor derived MSC population is an additive total score value obtained by addition of ranking score values for each individual donor derived MSC population.

57. Isolated, pooled allogeneic MSC population for use according to any one of items 1-55, wherein said total score value allocated to said each individual donor derived MSC population is a weighed total score value obtained by 1) assigning a weight to the ranking score value for each assay and 2) adding the weighed ranking score values for individual donor derived MSC population.

58. Isolated, pooled allogeneic MSC population for use according to any one of items 1-57, wherein the step of selecting a subset of individual donor derived MSC populations with desirable population properties comprises selecting at least 3, such as at least 4, such as at least 4, such as at least 5, such as at least 6, such as at least 7, such as at least 8, such as at least 9, such as at least 10 individual donor derived MSC populations.

59. Isolated, pooled allogeneic MSC population for use according to any one of items 1-58, in which population the number of cells derived from any one donor does not exceed 45%, such as does not exceed 40%, such as does not exceed 35%, of the total cell number and wherein said population comprises MSCs derived from at least 3 donors; such as in which population the number of cells derived from any one donor does not exceed 40%, such as does not exceed 35%, such as does not exceed 30%, of the total cell number and wherein said population comprises MSCs derived from at least 4 donors; such as in which population the number of cells derived from any one donor does not exceed 35%, such as does not exceed 30%, such as does not exceed 25%, of the total cell number and wherein said population comprises MSCs derived from at least 5 donors; such as in which population the number of cells derived from any one donor does not exceed 30%, such as does not exceed 25%, such as does not exceed 20%, of the total cell number and wherein said population comprises MSCs derived from at least 6 donors; such as in which population the number of cells derived from any one donor does not exceed 25%, as does not exceed 22 %, such as does not exceed 20 %, of the total cell number and wherein said population comprises MSCs derived from at least 7 donors.

60. Isolated, pooled allogeneic MSC population for use according to any one of items 1-59 in which population the number of MSC derived from any one donor does not exceed four times, such as three times, such as two times the number of the cells derived any other donor.

61. Isolated, pooled allogeneic MSC population for use according to item any one of items 1-60, said method further comprising the step of discarding an individual donor derived MSC population from the pooling step if the assay results for said individual donor derived MSC population are less desirable than the corresponding assay results for a pooled allogeneic MSC population previously obtained by the method as defined in any one of items 1-60.

62. Isolated, pooled allogeneic MSC population for use according to item any one of items 1-61, wherein said MSCs are obtained from a native MSC source.

63. Isolated, pooled allogeneic MSC population for use according to item any one of items 1-62,, wherein said pooled population exhibits enhanced immunosuppressive and/or immune-modulatory potential compared to individual donor derived MSC populations, such as each individual donor derived MSC population assayed, such as each individual donor derived MSC population selected for pooling.

64. Isolated, pooled allogeneic MSC population for use according to item 63,, wherein said enhanced immunosuppressive and/or immune-modulatory potential is measured as expression of IDO by unstimulated MSCs.

65. Isolated, pooled allogeneic MSC population for use according to item 63 or 64, wherein said enhanced immunosuppressive and/or immune-modulatory potential is measured as expression of PGE2 by unstimulated MSCs.

66. Isolated, pooled allogeneic MSC population for use according to any one of items 1 or 65,wherein said population exhibits no statistically significant batch-to-batch variability.

67. Isolated, pooled allogeneic MSC population for use according to any one of items 1-66, wherein said treatment and/or prevention of symptoms associated with COVID-19 infection is treatment and/or prevention the neurological symptoms associated with COVID-19 infection.

68. Isolated, pooled allogeneic MSC population for use according to any one of items 1-66, wherein said treatment and/or prevention of symptoms associated with long COVID-19 infection is treatment and/or prevention the neurological symptoms associated with long COVID-19 infection, such as treatment and/or prevention of inflammation and/or demyelination associated with long COVID-19 infection.

69. Isolated, pooled allogeneic MSC population for use according to any one of items 1-67, wherein said treatment and/or prevention of neurological symptoms associated with COVID-19 infection is treatment and/or prevention of inflammation and/or demyelination associated with COVID-19 infection.

70. Isolated, pooled allogeneic MSC population for use according to any one of items 1-69, wherein said use comprises administration of said MSC population as an infusion or injection to patient in need thereof.

71. Isolated, pooled allogeneic MSC population for use according to item 70, wherein said infusion or injection is administered intravenously, intraperitoneally, intralymphatically, intravenously, intrathecally, intracerebrally, intraarterially, subcutaneously or through the ommaya reservoir; such as intravenously, intraperitoneally or intralymphatically, such as intravenously.

72. Isolated, pooled allogeneic MSC population for use according to item 70 or 71, wherein said infusion or injection is administered intrathecally or intracerebrally or intravenously.

73. Isolated, pooled allogeneic MSC population for use according to any one of items 70-72, wherein said infusion is performed repeatedly.

74. Isolated, pooled allogeneic MSC population according for use according to any one of items 70-72, wherein said infusion performed one time only.

75. Isolated, pooled allogeneic MSC population for use according to any one of items 1-74, wherein said population after pooling has been exposed to a proinflammatory compound, such as IFNγ, tumor necrosis factor alpha and/or alum, for up to about 1 hour before administration or for between about 1 to about 24 hours before administration.

76. Isolated, pooled allogeneic MSC population for use according to any one of item 1-75, wherein administration of said MSC population induces no or low anti-HLA antibody titers in the patient.

77. Isolated, pooled allogeneic MSC population according for use according to any one of items 1-76, wherein said use comprises administration to said patient a dose of approximately at least 3 × 10⁶ cells, such as approximately at least 5 × 10⁶ cells, such as approximately at least 10 × 10⁶ cells, such as approximately at least 15 × 10⁶ cells, such as approximately at least 20 × 10⁶ cells, such as approximately at least 25 × 10⁶ cells, such as approximately at least 30 × 10⁶ cells, such as approximately at least 50 × 10⁶ cells, such as approximately at least about 60 × 10⁶ cells, such as approximately at least about 75 × 10⁶ cells, such as approximately at least about 100 × 10⁶ cells, such as approximately at least about 150 × 10⁶ cells, such as approximately at least about 200 × 10⁶ cells.

78. Isolated, pooled allogeneic MSC population according for use according to any one of items 1-77, wherein said uses comprises administration to said patient a dose of approximately at least 0.1 × 10⁶ cells/kg bodyweight, such as approximately at least 0,3 × 10⁶ cells/kg bodyweight, such as approximately at least 0,5 × 10⁶ cells/kg bodyweight, such as approximately at least 0,75 × 10⁶ cells/kg bodyweight, such as approximately at least 1 × 10⁶ cells/kg bodyweight, such as approximately at least 1,2 × 10⁶ cells/kg bodyweight.

79. Isolated, pooled allogeneic MSC population according for use according to any one of items 1-78, wherein said use comprises administering to said patient a dose from approximately 0.1 × 10⁶ cells/kg bodyweight to approximately 10 × 10⁶ cells/kg bodyweight, such as from approximately 0.15 × 10⁶ cells/kg bodyweight to approximately 4 × 10⁶ cells/kg bodyweight, such as from approximately 0.20 × 10⁶ cells/kg bodyweight to approximately 4 × 10⁶ cells/kg bodyweight, such as from approximately 0.25 × 10⁶ cells/kg bodyweight to approximately 4 × 10⁶ cells/kg bodyweight, such as from approximately 0.3 × 10⁶ cells/kg bodyweight to approximately 4 × 10⁶ cells/kg bodyweight, such as for example from approximately 0.25 × 10⁶ cells/kg bodyweight to approximately 3 × 10⁶ cells/kg bodyweight, such as from approximately 0.25 × 10⁶ cells/kg bodyweight to approximately 2 × 10⁶ cells/kg bodyweight or from approximately 0.3 × 10⁶ cells/kg bodyweight to approximately 1.2 × 10⁶ cells/kg bodyweight .

80. Pharmaceutical composition comprising an or an isolated, pooled allogeneic MSC population for use according to any one of items 1-79, and at least one pharmaceutically acceptable excipient or carrier.

81. Pharmaceutical composition according to item 80, comprising approximately at least 3 × 10⁶ cells, such as approximately at least 5 × 10⁶ cells, such as approximately at least 10 × 10⁶ cells, such as approximately at least 15 × 10⁶ cells, such as approximately at least 20 × 10⁶ cells, such as approximately at least 25 × 10⁶ cells, such as approximately at least 30 × 10⁶ cells, such as approximately at least 50 × 10⁶ cells, such as approximately at least about 60 × 10⁶ cells, such as approximately at least about 75 × 10⁶ cells, such as approximately at least about 100 × 10⁶ cells, such as approximately at least about 150 × 10⁶ cells, such as approximately at least about 200 × 10⁶ cells.

82. Pharmaceutical composition according to any one of items 7980, formulated for infusion; such for intravenous infusion, intraperitoneal infusion, intralymphatical infusion, intravenous infusion, intracerebral infusion, intrathecal infusion, intracerebral infusion, intraarterial infusion, subcutaneous infusion or infusion through the ommaya reservoir; such as for intracerebral or intrathecal infusion or such as for intravenous infusion.

83. Method for treatment and/or prevention of a disease or condition, which disease or condition is COVID-19 infection or is associated with COVID-19 infection, comprising administering a therapeutically effective dose of an isolated, pooled allogeneic MSC population according to any one of items 1-79 or a pharmaceutical composition according to any one of items 80-82, to a patient in need thereof.

84. Method for treatment and/or prevention of a disease or condition according to item 83 o wherein said disease or condition is neurological symptoms associated with COVID-19 infection.

85. Method for treatment and/or prevention of a disease or condition according to any one of items 83-84, wherein said disease or condition is inflammation and/or demyelination associated with COVID-19 infection.

86. Method for treatment and/or prevention according to any one of items 83-85, wherein said administration of said MSC population is by infusion; such as by intravenous infusion, intraperitoneal infusion, intralymphatical infusion, intravenous infusion, intrathecal infusion, intracerebral infusion, intraarterial infusion, subcutaneous infusion or infusion through the ommaya reservoir; such as by intrathecal infusion or intracerebral infusion or such as for intravenous infusion.

87. Method for treatment and/or prevention according to item 86, wherein said infusion is performed repeatedly.

88. Method for treatment and/or prevention according to item 86, wherein said infusion is performed one time only.

89. Method for treatment and/or prevention according to any one of items 83-88, wherein said population after pooling has been exposed to a proinflammatory compound, such as IFNγ, tumor necrosis factor alpha and/or alum, for between up to about 1 hour before administration or about 1 to about 24 hours before administration.

90. Method for treatment and/or prevention according to any one of items 83-89, wherein said administration induces no or low anti-HLA antibody titers in the patient.

91. Method for treatment and/or prevention according to any one of items 83-90, wherein said method comprises administering to said patient a dose of approximately at least 3 × 10⁶ cells, such as approximately at least 5 × 10⁶ cells, such as approximately at least 10 × 10⁶ cells, such as approximately at least 15 × 10⁶ cells, such as approximately at least 20 × 10⁶ cells, such as approximately at least 25 × 10⁶ cells, such as approximately at least 30 × 10⁶ cells, such as approximately at least 50 × 10⁶ cells, such as approximately at least about 60 × 10⁶ cells, such as approximately at least about 75 × 10⁶ cells, such as approximately at least about 100 × 10⁶ cells such as approximately at least about 150 × 10⁶ cells, such as approximately at least about 200 × 10⁶ cells.

92. Method for treatment and/or prevention according to any one of items 83-91, wherein said method comprises administering to said patient a dose of approximately at least 0.1 × 10⁶ cells/kg bodyweight, such as approximately at least 0,3 × 10⁶ cells/kg bodyweight, such as approximately at least 0,5 × 10⁶ cells/kg bodyweight, such as approximately at least 0,75 × 10⁶ cells/kg bodyweight, such as approximately at least 1 × 10⁶ cells/kg bodyweight, such as approximately at least 1,2 × 10⁶ cells/kg bodyweight.

93. Method for treatment and/or prevention according to any one of items 83-92, wherein said method comprises administering to said patient a dose from approximately 0.1 × 10⁶ cells/kg bodyweight to approximately 10 × 10⁶ cells/kg bodyweight, such as from approximately 0.15 × 10⁶ cells/kg bodyweight to approximately 4 × 10⁶ cells/kg bodyweight, such as from approximately 0.20 × 10⁶ cells/kg bodyweight to approximately 4 × 10⁶ cells/kg bodyweight, such as from approximately 0.25 × 10⁶ cells/kg bodyweight to approximately 4 × 10⁶ cells/kg bodyweight, such as from approximately 0.3 × 10⁶ cells/kg bodyweight to approximately 4 × 10⁶ cells/kg bodyweight, such as for example from approximately 0.25 × 10⁶ cells/kg bodyweight to approximately 3 × 10⁶ cells/kg bodyweight, such as from approximately 0.25 × 10⁶ cells/kg bodyweight to approximately 2 × 10⁶ cells/kg bodyweight or from approximately 0.3 × 10⁶ cells/kg bodyweight to approximately 1.2 × 10⁶ cells/kg bodyweight.

94. Use of an isolated, pooled allogeneic MSC population as defined in any one of items 1-79, in the manufacture of a medicament for the treatment of a disease or conditions selected from the group consisting COVID-19 infection and conditions associated with COVID-19 infection, such as neurological symptoms associated with COVID-19 infection, inflammation associated with COVID-19 infection and/or demyelination associated with COVID-19 infection.

95. Method for treatment and/or prevention of a disease or condition selected from the group consisting of COVID-19 infection and conditions associated with COVID-19 infection, such as neurological symptoms associated with COVID-19 infection, inflammation associated with COVID-19 infection and/or demyelination associated with COVID-19 infection, comprising the steps of
-obtaining an isolated, pooled allogeneic MSC population using the method as defined herein; and
-administering a therapeutically effective dose of said isolated, pooled allogeneic MSC population or of a pharmaceutical composition comprising said isolated, pooled allogeneic MSC population to a patient in need thereof.

1. A method for treatment and/or prevention of COVID-19 infection, or of symptoms associated with COVID-19 infection, comprising administering a therapeutically effective dose of isolated, pooled allogeneic MSC population comprises MSCs derived from at least 3 individual donors, wherein the number of cells derived from any one donor does not exceed 50% of the total cell number and wherein said MSCs have at most been subject to ten passages and wherein said isolated, pooled allogeneic MSC population is obtainable or is obtained by a method comprising the steps of:
culturing or providing MSCs from more than said at least 3 individual donors to obtain more than at least 3 individual donor derived MSC populations;
assaying each individual donor derived MSC population using at least 3 assays to obtain at least 3 assay results for said each individual donor derived MSC population;
for each assay allocating an individual ranking score value to said each individual donor derived MSC population based on the assay result and thus obtaining at least 3 individual ranking score values for each individual donor derived MSC population, wherein a higher ranking score value is indicative of more desirable assay result; or wherein a lower ranking score value is indicative of more desirable assay result;
allocating a total score value to each individual donor derived MSC population based on said at least 3 individual ranking score values, wherein in the case of a higher ranking score value being indicative of more desirable assay result, a higher total score value is indicative of more desirable population properties; or wherein in the case of a lower ranking score value being indicative of more desirable assay result, a lower total score value is indicative of more desirable population properties;
selecting a subset of individual donor derived MSC populations with desirable population properties based on their total score values; and
pooling said selected individual donor derived MSC populations to obtain an isolated, pooled allogeneic MSC population;
wherein at least 2 of said at least 3 assays are selected from the group consisting of one assay measuring indoleamine-2,3-dioxygensase (IDO) activity; one assay measuring prostaglandin E2 secreted by said MSCs; and one assay measuring the effect of said MSCs on the proliferation of peripheral blood mononuclear cells (PBMCs); and
wherein at least 1 of said at least 3 assays is selected from the group consisting of one assay measuring the effect of said MSCs on the capacity of T cells to suppress an immune response; one assay measuring the effect said MSCs on inducing tolerogenic dendritic cells, one assay measuring the effect of the said MSCs on monocytes; and one assay measuring the effect of the said MSCs on microglia cell and/or microglia-like cells: and
wherein said isolated pooled allogeneic MSC population is not further cultured after the pooling step.

2. (canceled)

3. The method for treatment and/or prevention of COVID-19 infection or of symptoms associated with COVID-19 infection according to claim 1, said method further comprising a step of exposing the isolated pooled allogeneic MSC population to the presence of proinflammatory factors.

4. The method for treatment and/or prevention of COVID-19 infection or of symptoms associated with COVID-19 infection according to claim 3, wherein the exposure is for a period of up to about 1 hour before administration or for between about 1 to about 24 hours before administration.

5. The method for treatment and/or prevention of COVID-19 infection or of symptoms associated with COVID-19 infection according to claim 1, wherein said MSCs are derived from a native MSC source.

6. The method for treatment and/or prevention of COVID-19 infection or of symptoms associated with COVID-19 infection according to claim 1, wherein said MSCs are selected from the group consisting of umbilical cord derived MSCs and Wharton Jelly derived MSCs.

7. The method for treatment and/or prevention of COVID-19 infection or of symptoms associated with COVID-19 infection according to claim 1, wherein said population comprises MSCs derived from at least four individual donors .

8. The method for treatment and/or prevention of COVID-19 infection or of symptoms associated with COVID-19 infection according to claim 1, wherein said method comprises one assay measuring the effect of the said MSCs on microglia cells or microglia-like cells and said assay is selected from the group consisting of one assay measuring microglial proliferation; one assay measuring expression of markers characteristic of the M1 phenotype in microglia; one assay measuring expression of markers characteristic of the M2 phenotype in microglia; and an assay measuring the shift from the M1 microglia phenotype to the M2 microglia phenotype.

9. The method for treatment and/or prevention of COVID-19 infection or of symptoms associated with COVID-19 infection according to claim 1,wherein said pooled population exhibits enhanced immunosuppressive and/or immune-modulatory potential compared to individual donor derived MSC population .

10. The method for treatment and/or prevention of COVID-19 infection or of symptoms associated with COVID-19 infection according to claim 1, wherein said immunosuppressive and/or immune-modulatory potential is measured as expression of IDO by unstimulated MSCs.

11. The method for treatment and/or prevention of COVID-19 infection or of symptoms associated with COVID-19 infection according to claim 9, wherein said immunosuppressive and/or immune-modulatory potential is measured as expression of PGE2 by unstimulated MSCs.

12. The method for treatment and/or prevention of COVID-19 infection or of symptoms associated with COVID-19 infection according to claim 9, wherein the enhancement of immunosuppressive and/or immune-modulatory potential is by at least approximately 5 % .

13. The method for treatment and/or prevention of COVID-19 infection or of symptoms associated with COVID-19 infection according to claim 1, wherein said treatment and/or prevention of symptoms associated with COVID-19 infection is treatment and/or prevention of neurological symptoms associated with COVID-19 infection.

14. The method for treatment and/or prevention of COVID-19 infection or of symptoms associated with COVID-19 infection according to claim 1, wherein said treatment and/or prevention of neurological symptoms associated with COVID-19 infection is treatment and/or prevention of inflammation and/or demyelination associated with COVID-19 infection.

15. The method for treatment and/or prevention of COVID-19 infection or of symptoms associated with COVID-19 infection according to claim 1, wherein said treatment and/or prevention of symptoms associated with COVID-19 infection is treatment and/or prevention of symptoms associated with long COVID-19 infection .

16. The method for treatment and/or prevention of COVID-19 infection or of symptoms associated with COVID-19 infection according to claim 1, wherein said use comprises exposing the isolated pooled allogeneic MSC population to the presence of proinflammatory factors prior to administration .

17. The method for treatment and/or prevention of COVID-19 infection or of symptoms associated with COVID-19 infection according to claim 1, wherein said use comprises administration by intravenous infusion or intravenous injection.

18. (canceled)

19. The method of claim 6, wherein the MSCs are Wharton Jelly derived MSCs.

20. The method of claim 7, wherein the population comprises MSCs derived from at least at least five individual donors, at least six individual donors, at least seven individual donors, at least eight individual donors, at nine individual donors, or at least ten individual donors.

21. The method of claim 9, wherein the individual donor derived MSC population comprises an individual donor derived MSC population assayed or an individual donor derived MSC population selected for pooling.

22. The method of claim 12, wherein the enhancement of immunosuppressive and/or immune-modulatory potential is by at least approximately 10 %, or at least by approximately 15 %, as compared to individual donor derived MSC populations.


SEQUENCE LISTING (6 sequences)

SEQ ID NO: 1 - DNA, 20 bp, Homo sapiens
ttctgtcacccgattttcaa

SEQ ID NO: 2 - DNA, 19 bp, Homo sapiens
tcccaacaatcgtgagtgc

SEQ ID NO: 3 - DNA, 18 bp, Homo sapiens
cgctggcctactctggaa

SEQ ID NO: 4 - DNA, 21 bp, Homo sapiens
ctgaggagtcgcactctatcc

SEQ ID NO: 5 - DNA, 23 bp, Homo sapiens
cctggaggagaagaggaaagaga

SEQ ID NO: 6 - DNA, 25 bp, Homo sapiens
ttgaggacctctgtgtatttgtcaa